SEQUENCE LISTING (4 sequences)

SEQ ID NO: 1 - DNA, 35 bp, Homo sapiens
agacgttgccctcgaggtcaatgttggggtgatag

SEQ ID NO: 2 - DNA, 17 bp, Artificial Sequence
  mutated UBE2M sequence
agacgttggggtgatag

SEQ ID NO: 3 - DNA, 20 bp, Artificial Sequence
  DNA sequence encoding sgUBE2M RNA
tcaccccaacattgacctcg

SEQ ID NO: 4 - DNA, 20 bp, Artificial Sequence
  DNA encoding sgCUL4B RNA
agcatgtggtacttactggg

(19) United States
(12) Patent Application Publication
(10) Pub. No.: US 2024/0132485 A1
(43) Pub. Date: Apr. 25, 2024

(54) HETEROCYCLIC CULLIN RING UBIQUITIN LIGASE COMPOUNDS AND USES THEREOF

(71) Applicants: CEMM - FORSCHUNGSZENTRUM FÜR MOLEKULARE MEDIZIN GMBH, Vienna (AT); PROXYGEN GMBH, Vienna (AT)

(72) Inventors: Georg WINTER, Vienna (AT); Cristina MAYOR RUIZ, Zaragoza (AT); Stefan KUBICEK, Vienna (AT); Alastair David Graham DONALD, Wuppertal (DE); Grasilda ZENKEVICIUTE

(21) Appl. No.: 18/249,114

(22) PCT Filed: Oct. 15, 2021
(86) PCT No.: PCT/EP2021/078696

§ 371 Date: Apr. 14, 2023

(30) Foreign Application Priority Data
Oct. 16, 2020 (EP) 20 202 417.0

(51) Int. Cl. C07D 417/12; A61P 31/02; C07D 213/75; C07D 405/12; C07D 417/14; C07D 471/04

(52) U.S. Cl. CPC C07D 417/12; A61P 31/02; C07D 213/75; C07D 405/12; C07D 417/14; C07D 471/04

(57) ABSTRACT

The present invention relates to compounds with the ability to stimulate/induce ubiquitination of a target protein/target proteins. The compounds of the present invention may stimulate/induce ubiquitination of a target protein/target proteins; i.e. via degradation of a target protein/target proteins by the cullin-RING ubiquitin ligase (CRL). Such target protein/target proteins may be proteins involved in diseases, like cancer, metabolic disorder, infectious disease and/or neurological disorder. The invention also relates to the compounds and composition for use as medicaments as well as pharmaceutical compositions comprising these compounds. Particularly, the compounds of the present invention may degrade proteins associated with cancer, metabolic disorder, infectious disease and/or neurological disorder. Furthermore, the present invention relates the compounds for use as a medicament, such as for use in treating cancer, metabolic disorder, infectious disease and/or neurological disorder and to a method for treating a disease, such as cancer, metabolic disorder, infectious disease and/or neurological disorder, comprising administering the compound of the present invention.

### FIELD OF THE INVENTION

The present invention relates to compounds with the ability to modulate/stimulate/induce, particularly induce ubiquitination of a target protein/target proteins. The compounds of the present invention may stimulate/induce ubiquitination of a target protein/target proteins; i.e. via degradation of a target protein/target proteins by the cullin-RING ubiquitin ligase (CRL). Such target protein/target proteins may be proteins involved in diseases, like cancer, metabolic disorder, infectious disease and/or neurological disorder. The invention further relates to a method for identifying/obtaining and/or testing a compound able to induce ubiquitination of a target protein/target proteins. The invention also relates to the compounds and composition for use as medicaments as well as pharmaceutical compositions comprising these compounds. Particularly, the compounds of the present invention may facilitate degradation of proteins associated with cancer, metabolic disorder, infectious disease and/or neurological disorder. Furthermore, the present invention relates the compounds for use as a medicament, such as for use in treating cancer, metabolic disorder, infectious disease and/or neurological disorder and to a method for treating a disease, such as cancer, metabolic disorder, infectious disease and/or neurological disorder, comprising administering the compound of the present invention.

### BACKGROUND OF THE INVENTION

Protein degradation plays a central role in many cellular functions such as for cell maintenance and normal function. Accordingly, degradation of proteins, such as proteins which are associated with cellular functions, e.g., maintenance function, has implications for the cell's proliferation, differentiation, and death. In this context, the chemical induction of targeted protein degradation (TPD), thereby reducing the activity of a protein by removing the target protein, is a highly promising paradigm in drug discovery compared to inhibitors of proteins which would reduce the activity of a protein by simply blocking said protein. Utilizing a cell's protein degradation pathway can, therefore, provide means for reducing or removing protein activity.

Until recently, small molecules that induce protein destabilization typically emerged serendipitously. Examples for this are the estrogen receptor (ER) modulator Fulvestran, or the CRL4^{CRBN} modulators thalidomide and related compounds such as lenalidomide or pomalidomide (collectively referred to as “IMiDs” and also known in the art as “molecular glue”). All these cases represent approved drugs, which clinically validates the concept of TPD as a therapeutic reality. Lenalidomide was, in fact, with total revenues of $9.7 billion, one of the commercially most successful drugs of 2018.

Noteworthy, it took several decades of research to decipher the molecular mechanism of IMIDs as small molecule degraders. Rational strategies to generalize the concept of TPD were described by Winter et al. (Winter, G. E.*, Buckley, D. L.*, Paulk, J., Roberts, J., Souza, A., De-Phagano, S., and Bradner, J. E. (2015) Phthalimide Conjugation as a Strategy for in vivo Target Protein Degradation. Science 348, 1376-81), describing the formation of heterobifunctional molecules by conjugating IMiD-like chemical structures to known targeting ligands via flexible linkers. These heterobifunctional small molecules (often also called “degraders”) are shown to function via binding to a protein of interest (via the interchangeable targeting ligand) and the E3 ligase CRL4C^{RBN}, i.e. via the IMiD-like chemical agent. Thereby, binding induces molecular proximity between the target protein and the E3 ligase, prompting ubiquitination and proteolytic degradation of the former. Particularly, the ubiquitin conjugation on target proteins is mediated by an enzymatic cascade comprised by an E1 ubiquitin-activating enzyme, an E2 ubiquitin-conjugating enzyme and an E3 ubiquitin ligase that attach ubiquitin to the target protein(Hershko et al., Nat. Med. 6, 1073-1081 (2000); Komander et al., Annu. Rev. Biochem. 81, 203-229 (2012)).

Thus, the ubiquitin-proteasome pathway, one of the cell's major degradation pathways and which is a critical pathway that regulates key regulator proteins and degrades misfolded and abnormal proteins, is found to be a valuable tool, in particular in therapeutic applications, for degrading target proteins by covalent attachment of ubiquitin to the said target protein.

The development of heterobifunctional degraders (PROTAC) that have the ability to hijack the CRBN ligase complex is associated with certain caveats. For example, only certain E3 ligases can be harnessed by such heterobifunctional degraders. Thereby, ligands typically bind to CRBN, VHL, cIAIP or MDM2. Furthermore, a part of the heterobifunctional degrader structure of PROTACs is a ligand to the target protein, thereby precluding the application of the technology to “unligandable” proteins (see, e.g., Surade and Blundell (2012); Chemistry & Biology, Volume 19, Issue 1, pp. 42-50). Sometimes, the high molecule weight of the resulting heterobifunctional degraders may impact pharmacology and bioavailability.

There is a need for efficient small molecules that are able to bind to E3 ligase components, and which are thus suitable to be to degrade desired target proteins.

Small molecules may modulate E3 ligases and other components of the ubiquitin-proteasome pathway by operating via a “molecular glue” type of mechanism. By this means, such compounds may not rely on the availability of an accessible, hydrophobic binding pocket. For example, IMiDs can induce cooperative associations with target proteins that are naturally not bound by CRBN, i.e. without requiring an additional linkage with a targeting-moiety. This in turn prompts ubiquitination and proteasomal degradation of bound target proteins such as the transcription factors IKZF1 and IKZF3. As another example, aryl sulfonamides can re-direct the activity of the E3 ligase DCAF15 to degrade the splicing factor RBM39 in an analogous manner as IMiDs. Similarly, the phytohormone auxin is known to re-direct the target space of the E3 ligase Tir1 to induce degradation of the Aux/IAA transcriptional repressors.

Until now, targeting proteins which are devoid of a hydrophobic binding pocket or a binding site that leads to inactivation of said target proteins are beyond the reach of commonly used compounds which may be developed for therapeutic uses. In other words, this approach does not allow degradation of target proteins, such as target proteins without an accessible hydrophobic pocket or inhibitory binding site. In this regard, compelling disease-relevant targets such as MYC, RAS, or b-catenin, remain beyond the reach of therapeutic development.

Thus, novel paradigms in drug design are highly needed. Hence, in view of the above, the technical problem underlying the present invention is the provision of compounds and methods for identifying compounds that are able to induce ubiquitination of a target protein/target proteins, in particular a target protein/target proteins desired to be degraded in a cell, like a diseased cell.

The solution to this technical problem is provided by the embodiments as defined herein below and as characterized in the claims.

### SUMMARY OF THE INVENTION

The invention relates to the compounds of formulae (I), (II), (III), (IV) and (V) as described herein as well as to their use in the treatment of various diseases which can be treated by targeted degradation of certain proteins.

The compounds as disclosed herein and in context of the invention are capable of modulating/stimulating/inducing ubiquitination of a target protein/target proteins, e.g. via degradation of a target protein/target proteins by the ubiquitination system. In context of the invention, the compound has the capacity of modulating/stimulating/inducing, particularly inducing ubiquitination of a target protein/target proteins by enhancing the cullin-RING ubiquitin ligase activity/CRL activity.

The compounds as disclosed herein and in context of the invention may particularly be used as molecular glues as described herein and illustrated in the appended Examples. The compounds of the invention may also be envisaged to be used for the development of heterobifunctional molecules, such as PROTAC®s (proteolysis targeting chimera).

Accordingly, it is envisaged that the compounds of the present invention can be used as building blocks for the development of heterobifunctional molecules, such as PROTAC®s. When being used as building blocks for the development of PROTAC®s, it is preferred that the compounds of the present invention wherein are attached to the rest of the PROTAC® by the formation of a covalent bond between the compounds of the present invention (such as the compound of formulae (I), (II), (III), (IV) and (V)) and the rest of the PROTAC®. The skilled person is aware of suitable synthetic methods for forming bonds between two molecules. Coupling reactions of various types are known in synthetic organic chemistry, such as set out in “Cross-Coupling Reactions—A Practical Guide” 2002 by N. Miyaura, ISBN 978-3-540-45313-0. The terms “PROTAC®”, “PROTAC™”, “PROTAC”, “PROTAC®s”, “PROTACTMs”, “PROTACs” or “proteolysis targeting chimera” are used interchangeably and refer in particular to heterobifunctional compounds. As also described herein, PROTACs are known to the person skilled in the art to have advantageous properties such as but not limited to their interchangeable target binding moiety which can bind to a desired target to be degraded. However, certain protein(s) to be degraded are considered “unligandable” and are therefore not degradable by PROTACs. Such “unligandable” protein(s) (yet desired to be degraded) cannot be degraded via the PROTAC mechanism because no target binding moiety (moieties) for the “unligandable” protein(s) are known or available. “Unligandable” proteins are known in the art and include, inter alia, those having featureless binding sites, lack of hydrogen-bind donors and acceptors, the need for adaptive changes in conformation, and the lipophilicity of residues at the protein-ligand interface; see, e.g., Surade and Blundell (2012); Chemistry & Biology, Volume 19, Issue 1, pp. 42-50. Accordingly, and as described herein, the compounds of the present invention, however, can be of advantage because they are able to modulate/induce/stimulate degradation of “unligandable” protein(s), for example as “molecular glue”.

Molecular glues are capable of degrading target protein(s) by orchestrating direct interactions between target and cullin-RING ligases (CRLs). Molecular glues have the potential to induce the elimination of disease-relevant proteins otherwise considered “undruggable”. The mechanism of action by molecular glues can be exemplified by the clinically approved molecular glues/degraders of thalidomide analogs (IMiDs). Binding of IMiDs to the CRL4CRBN E3 ligase causes recruitment of selected zinc finger transcription factors (TFs), leading to their ubiquitination and subsequent proteasomal degradation (Lu, G. et al. Science 343, 305-309, doi:10.1126/science.1244917 (2014); Kronke, J. et al. Science 343, 301-305, doi:10.1126/science.1244851 (2014); Sievers, Q. L. et al. Science 362, doi:10.1126/science.aat0572 (2018); Gandhi, A. K. et al. British journal of haematology 164, 811-821, doi:10.1111/bjh.12708 (2014)).

Noteworthy, IMiDs have per se no measurable binding affinity to the degraded TFs. However, they orchestrate molecular recognition between ligase and TF by inducing several protein-protein interactions proximal to the binding interface. Certain aryl sulfonamides around the clinically tested compound indisulam act as molecular glues between the CRL4^{DCAF15} ligase and the splicing factor RBM39, causing the targeted degradation of the latter (Han, T. et al., Science, doi:10.1126/science.aal3755 (2017); Uehara, T. et al. Selective degradation of splicing factor CAPERalpha by anticancer sulfonamides. Nat Chem Biol 13, 675-680, doi:10.1038/nchembio.2363 (2017); Bussiere, D. E. et al. Nat Chem Biol 16, 15-23, doi:10.1038/s41589-019-0411-6 (2020); Ting, T. C. et al. Cell reports 29, 1499-1510.e1496, doi:10.1016/j.celrep.2019.09.079 (2019); Faust, T. B. et al. Nat Chem Biol 16, 7-14, doi:10.1038/s41589-019-0378-3 (2020); Du, X. et al. Structure (London, England: 1993) 27, 1625-1633.e1623, doi:10.1016/j.str.2019.10.005 (2019).)

The molecular glue mechanism of action therefore enables the destabilization of target proteins otherwise considered “unligandable” and thus outside the reach of both traditional small-molecule inhibitors and also of heterobifunctional degraders.

The compounds of the invention are able to induce the destabilization of disease associated target proteins, such as cyclin K (CCNK), CDK12 and/or CDK13. The compounds of the invention act, inter alia, as CCNK degraders. As described herein and illustrated in the appended Examples, the compounds of the invention are able to degrade target protein(s), such as cyclin K (CCNK), CDK12 and/or CDK13, independent of a dedicated substrate receptor, which functionally differentiates this mechanism from previously characterized degraders.

As discussed above, the compounds of the invention may also be envisaged to be used in heterobifunctional molecules, such as PROTACs. The term “PROTAC®” is used interchangeably and refers to heterobifunctional compounds as used herein refer to a compound that induce proteasome-mediated degradation of selected proteins via their recruitment to E3 ubiquitin ligase and subsequent ubiquitination (Crews C, Chemistry & Biology, 2010, 17(6):551-555; Schnnekloth J S Jr., Chembiochem, 2005, 6(1):40-46). The term refers to proteolysis-targeting chimera molecules having generally three components, an E3 ubiquitin ligase binding group (i.e. an E3 Ligase Binding Moiety (EBM)), optionally a linker (L), and a protein binding group of a target (i.e. a target binding moiety (TBM)). A PROTAC/proteolysis-targeting chimera may be illustrated by the following formula:

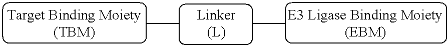

wherein TBM is a moiety binding to a target protein,
preferably wherein the TBM is a moiety binding to a target protein associated with cancer, metabolic disorders, neurologic disorders or infectious diseases;
more preferably wherein the one or more protein(s) associated with cancer is selected from the group consisting of DNA-binding proteins including transcription factors such as ESR1, AR, MYB, MYC; RNA binding proteins; scaffolding proteins; GTPases such as HRAS, NRAS, KRAS; solute carriers; kinases such as CDK4, CDK6, CDK9, EGFR, SRC, PDGFR, ABL1, HER2, HER3, BCR-ABL, MEK1, ARAF, BRAF, CRAF, phosphatases, bromodomain- and chromodomain containing proteins such as BRD2, BRD3, BRD4, CBP, p300, ATAD2, SMARCA2, SMARCA4, PBRM1, G-protein coupled receptors; anti-apoptotic proteins such as SHP2, PTPN1, PTPN12; immune regulators such as PDL1 and combinations thereof,
even more preferably wherein the one or more protein(s) associated with cancer is selected from the group consisting of BRD2, BRD3, BRD4, CBP, p300, ATAD2, SMARCA2, SMARCA4, PBRM1, CDK4, CDK6, CDK9, CDK12 and/or CDK13, EWS-FLI, CDC6, CENPE, EGFR, SRC, PDGFR, ABL1, HER2, HER3, BCR-ABL, MEK1, ARAF, BRAF, CRAF, HRAS, NRAS, KRAS, BCL2, MCL2, SHP2, PTPN1, PTPN12, ESR1, AR, MYB, MYC, PDL1 and combinations thereof;
even more preferably wherein the one or more protein(s) associated with cancer are selected from the group consisting of KRAS, NRAS, MYC, MYB, ESR1, AR, EGFR, HER2, BCR-ABL1 and BRAF;
most preferably the one or more protein(s) associated with cancer are selected from the group consisting of KRAS, NRAS, MYC and MYB.
more preferably wherein the one or more protein(s) associated with metabolic disorders are selected from the group consisting of ARX, SUR, DPP4 and SGLT;
more preferably wherein the one or more protein(s) associated with neurologic disorders are selected from the group consisting of Tau and beta-amyloid; and
wherein the one or more protein(s) associated with infectious diseases are selected from the group consisting of CCR5 and PLA2G16:
wherein L is a linker moiety; and
wherein EBM is a moiety modifying the function of the E3 ligase and/or binding to at least one regulator or member of the E3 ligase complex;
preferably wherein the at least one member of the E3 ligase complex (CRL) is selected from the group consisting of CUL4B; DDB1; RBX1; UBE2G1; and CUL4A; and wherein the at least one regulator of the E3 ubiquitin ligase complex is selected from the group consisting of, UBE2M; UBA3; UBE2F; NAE1;COPS1, COPS2, COPS3, COPS4, COPS5, COPS6, COPS7A, COPS7B, COPS8; DCUN1D1; DCUN1D2; DCUN1D3; DCUN1D4; DCUN1D5;
more preferably wherein the at least one member or regulator of the E3 ubiquitin ligase complex is CUL4B or DDB1;
even more preferably wherein the EBM is comprised in a structure selected from the group consisting of any of compounds of formulae (I), (II), (III), (IV) and (V).

It is to be understood that when the EBM comprises a structure selected from the group consisting of any of compounds of formulae (I), (II), (III), (IV) and (V), the TBM-L-EBM structure indicated above is formally obtained by establishing a bond between the linker moiety (which is preferably also connected to the TBM) and the EBM comprising the structure selected from any of compounds of formulae (I), (II), (III), (IV) and (V), e.g. by formally removing a hydrogen radical from both the linker and the compound selected from any compound of formulae (I), (II), (III), (IV) and (V) belonging to the EBM and combining the thus hypothetically obtained radical of the linker with the radical of the structure comprising the compound selected from any compound of formulae (I), (II), (III), (IV) and (V) belonging to the EBM so as to form a bond between the two atoms hypothetically having born the two radicals, respectively. Preferably, the EBM is a structure selected from the group consisting of any of compounds of formulae (I), (II), (III), (IV) and (V).

Said “target protein” is, in particular a target protein desired to be degraded in particular via (an) ubiquitination(s). The term “target protein” as used in this context also comprises a plurality of proteins of target proteins. This is also illustrated in the appended examples. In one embodiment, the “target protein” in context of this invention is a protein which is desired or is desirable to be degraded in an in vivo or in vitro situation, for example in a diseased cell, like a cancer cell. Particular target proteins are, in one specific embodiment, proteins that are the cause, the driver and/or the maintaining entity of a malignancy, disease, or a diseased status. Such target proteins may comprise proteins that are overexpressed and/or overactive in a diseased cell, like in a cancer cell. Accordingly, in one embodiment, the target protein is involved in the cause, development and/or maintenance of the diseased status of a cell and/or a tissue. Potential target proteins are also discussed herein below and illustrative, non-limited examples are provided herein below. Target protein(s) as described herein may be degraded via direct or indirect binding to a compound of the invention. Particular examples of such target proteins are, but are not limited to, CDK12, CDK13 and/or CCNK. In this context, CDK12, CDK13 and/or CCNK may be desired or desirable to be degraded in an in vivo or in vitro situation, for example in a diseased cell, like a cancer cell. Thus, the target protein(s) as disclosed herein and in the context of the invention may be target protein(s) associated with cancer, wherein the one or more protein(s) associated with cancer may be selected from the groups consisting of CDK12, CDK13 and CCNK. As another particular example, the target protein may be a target protein associated with cancer, wherein the one or more protein(s) associated with cancer may be kinases, such as CDK12 and/or CDK13.

For example, said compound may facilitate the recognition of a target protein by the E3 ligase complex or may facilitate ubiquitination even without physically engaging the target protein at the same time. The compound may also enable said recognition of a target protein by the E3 ligase complex. A further non-limiting option of the “induction of ubiquitination of a target protein” may comprise the conformational change of the target protein that has been induced as a direct consequence of binding/interaction with said compound inducing the ubiquitination of the target protein. For example, binding of a compound as described herein to a target protein may lead to a conformational change of said protein and thereby stabilize an interaction of one or more target protein(s) with one or more component(s) of the E3 ligase complex that results in ubiquitination and degradation of said one or more target protein(s). Particularly, a compound binding to CDK12/13:CCNK prompts interaction with a DDB1:CUL4B E3 ligase complex, leading to the ubiquitination and degradation of CCNK. By this means, a target protein as described herein and illustrated in the appended examples, such as CCNK, may be degraded via a direct or an indirect binding mechanism of a compound as described herein, such as by binding of said compound to a protein associated with a target protein. A compound may bind to CDK12/13, which is associated with CCNK, thereby leading to the ubiquitination and degradation of CCNK. This interaction is independent from a particular substrate receptor of an E3 ligase. Thus, a compound as described herein and in context of the invention can degrade one or more target protein(s) via an E3 ligase independent of a particular substrate receptor of said E3 ligase.

In particular, the compounds of the present invention may bind in particular to the active site of CDK12/13, thereby prompting a change in structural conformation, which promotes the binding of CDK12:CCNK and CDK13:CCNK, respectively, to DDB1:CUL4B. As such, CDK12 and CDK13 basically serve to present CCNK to the ligase, leading to the degradation of, among others, CCNK, followed by a potentially slightly weaker degradation of CDK12 and CDK13.

Said “enhanced cullin-RING ubiquitin ligase activity”/“enhanced CRL activity” means that said cullin-RING ubiquitin ligase activity/CRL activity is enhanced in the presence of the compound of the present invention compared to the cullin-RING ubiquitin ligase activity/CRL activity in the absence of said compound. Accordingly, the present invention relates to a compound with the capacity to induce and/or stimulate the ubiquitination of a target protein/target proteins via enhancing the CRL activity. The cullin-RING ubiquitin ligase activity/CRL activity may be determined by methods known in the art and provided below.

The enhanced CRL activity is induced by the presence of said compound. Said compound may be able to induce molecular proximity between a component of a E3 ligase complex/cullin-RING ubiquitin ligase complex/CRL complex and a target protein/target proteins which may be bound to the compound or which may be part of a ternary complex comprising the E3 ligase complex/cullin-RING ubiquitin ligase complex/CRL complex, the target protein/target proteins and the compound. The compound of the present invention may bind a target protein/target proteins via the target binding moiety/TBM of the compound and bind or modify the function of the E3 ligase complex/cullin-RING ubiquitin ligase complex/CRL complex, for example by recruiting the target protein/target proteins bound to the target binding moiety/TBM of the compound/the compound to the E3 ligase complex/cullin-RING ubiquitin ligase complex/CRL complex. For example, the compound may bind to at least one member of the E3 ligase complex/cullin-RING ubiquitin ligase complex/CRL complex and the target protein. As another example, the compound in context of the invention may alter the function of a target protein, for example by modifying posttranslational changes of a target protein. A posttranslational modification may include but is not limited to the phosphorylation status of a protein, e.g. a tyrosine kinase phosphorylating a protein. Thus, the compound may induce ubiquitination of a target protein, e.g., by modifying a target protein in that the target protein becomes accessible for a E3 ligase complex/cullin-RING ubiquitin ligase complex/CRL complex, thereby the compound may not associate with a target protein and/or E3 ligase complex/cullin-RING ubiquitin ligase complex/CRL complex.

The target protein/target proteins may be ubiquitinated by the E3 ligase complex/cullin-RING ubiquitin ligase complex/CRL complex. Particularly, the inventors found that target proteins including those devoid of a hydrophobic binding pocket and/or inhibitory binding site can be recognized by the compounds of the present invention. Such target proteins may further include proteins which are not recognized E3 ligase complex/cullin-RING ubiquitin ligase complex/CRL complex in the absence of the compound of the present invention. Thus, it has been surprisingly found that the compounds of the present invention are able to induce ubiquitination of the target protein/target proteins, i.e. via degradation of the target protein/target proteins by the ubiquitination system.

### DESCRIPTION OF THE INVENTION

Several target proteins involved in the cause, development and/or maintenance of a diseased status are devoid of obvious ligand-binding sites, for example inhibitory binding sites, or hydrophobic pockets. Such target proteins include but are not limited to transcription factors, such as the zinc-finger transcription factors IKZF1 and IKZF3, which are devoid of hydrophobic pockets. As another example, such target proteins may include but is not limited to CDK12, CDK13 and/or CCNK. As yet another example, such target protein(s) may include but are not limited to kinases such as CDK12 and/or CDK13. Moreover, target proteins which may not comprise a binding site that results in an altered function of said target protein, such as inhibition or activation upon binding of a compound to said binding site, are “undruggable” drug targets because compounds directed to target proteins involved in the cause, development and/or maintenance of a diseased status comprise compounds that recognize hydrophobic binding pockets and/or a binding site altering the function of said target protein.

Compounds which may act via ubiquitination of the target protein, thereby degrading the target protein by the ubiquitination system could overcome these limitations by connecting a component of the E3 ligase and target protein. These molecules could orchestrate novel interactions between a component of the E3 ligase and a target protein at the dimerization interface to form a trimeric complex comprising the component of the E3 ligase, the molecule and the target protein.

For example, such compounds may be molecular glues as described herein and used in context of the invention. As described herein and illustrated in the appended Examples, said molecular glues are able to degrade “undruggable” and/or “unligandable” proteins.

As used herein and as also discussed herein above, the term “unligandable” refers to a protein that cannot be bound by ligands and/or that does not possess a binding site suitable for binding of said unligandable protein with a ligand. For example, whether a target protein is unligandable may be determined using a structure-based algorithm, wherein the capability of binding of ligands to a protein is assessed based on parameters computed for binding pockets on a protein including parameters such as but not limited to volume, surface area, lipophilic surface area, depth and/or hydrophobic ratio.

As used herein, the term “undruggable” refers to a protein refers to a protein that cannot be bound by a drug compound and/or that does not possess a binding site suitable for binding of said undruggable protein with a drug compound. Thus, an undruggable protein refers to a protein which does not successfully interfere with a drug compound (e.g. a ligand such as an antibody) used in therapy. Therefore, typically, an undruggable protein may be a protein that lacks a binding site for a drug compound or for which, despite having a binding site, successful targeting of said site has proven intractable.

Further, molecular glues as described herein and illustrated in the appended Examples may degrade one or more target protein(s) via interaction with a component of the cullin RING E3 ligase present in several family members of the cullin RING E3 ligase. Particularly, the family members of the cullin RING E3 ligase can be diversified, e.g., by their respective substrate receptors, such as CRBN or DCAF15. The compounds, in particular molecular glues, as described herein can bind to components of the cullin RING E3 ligase family other than the substrate receptor, and thus these compounds may degrade one or more target protein(s) independent from the substrate receptor. Thus, the ability of molecular glues to degrade one or more target protein(s) via interaction with a cullin RING E3 ligase may not be limited to a particular family member of a cullin RING E3 ligase.

For example, a molecular glue as described herein may degrade one or more target protein(s) associated with cancer, such as CDK12, CDK13 and/or cyclin K (CCNK). Thereby, the mechanism of action by molecular glues resulting in degradation of one or more target protein(s) such as CDK12, CDK13 and/or cyclin K (CCNK), can be due to the ability of molecular glues to orchestrate protein-protein interactions between a cullin RING E3 ligase and one or more target protein(s) to be degraded. As described herein, this can be achieved by stabilizing an interaction of CDK12 and/or CDK13 bound to CCNK with the cullin RING E3 ligase, particularly one or more components of the cullin RING E3 ligase such as CUL4B and/or DDB1.

In this context, the present invention provides novel compounds that stimulate/induce ubiquitination of a target protein/target proteins, i.e. via target protein degradation by the cullin RING E3 ligase, wherein the compound has any one of formulae (I), (II), (III), (IV) and (V) as described herein.

In context of the invention, the compounds are particularly useful as medicaments, for example in the treatment of diseases and/or disorders wherein it is desired to degrade target protein/target proteins via ubiquitination. Accordingly, the present invention also provides for methods of treating such diseases or disorders, said methods comprising the administration to an individual in need of such a treatment with the compound of the invention, i.e. the compound that can stimulating/induce ubiquitination of a target protein/target proteins. Particularly, the inventive compounds provided herein are used in biochemical degradation of misfolded and/or abnormal proteins in vivo as well as in vitro.

In the following, examples of compounds of the present invention (in particular of formulae (I), (II), (III), (IV) and (V)) are presented. It is to be understood that these also encompass any stereoisomers, tautomers, pharmaceutically acceptable salts, solvates and prodrugs of the compounds presented as Markush formulae or specific formulae.

The term “molecular glue” is generally known in the art and refers to a compound that can bind at least two different molecules at a time by cooperative binding but has no binding affinity to one of the at least two different molecules separately. In other words, a molecular glue refers to a compound that binds to a target protein/target proteins the compound simultaneously binds to the target protein/target proteins and a second protein. In context of the invention, a molecular glue refers to a compound that binds to a target protein/target proteins if the compound may simultaneously bind to the target protein/target proteins and at least one member or regulator of the E3 ligase complex. Examples for molecular glues known in the art include but are not limited to non-chimeric small molecules, lenalidomide, pomalidomide, CC-885 and related immunomodulatory drugs (IMiDs). The compounds of the invention may comprise molecular glues that bind to a target protein/target proteins if the compound may simultaneously bind to the target protein/target proteins and at least one member or regulator of the E3 ligase complex. Such molecular glues of the invention are further described herein below and are illustrated by the appended Examples.

The compounds of the invention may also comprise PROTAC®s (proteolysis targeting chimera). The term “PROTAC®”, “PROTAC®s” or “proteolysis targeting chimera” is used interchangeably and refers to heterobifunctional compounds as used herein refer to compound that induce proteasome-mediated degradation of selected proteins via their recruitment to E3 ubiquitin ligase and subsequent ubiquitination (Crews C, Chemistry & Biology, 2010, 17(6):551-555; Schnnekloth J S Jr., Chembiochem, 2005, 6(1):40-46). In other words, this term refers to proteolysis-targeting chimera molecules having generally three components, an E3 ubiquitin ligase binding group, optionally a linker, and a protein binding group of a target. Phthalimide conjugation as a strategy for in vivo target protein degradation. Science 348, 1376-1381 (2015), Bondeson, D. P. et al. Catalytic in vivo protein knockdown by small-molecule PROTAC®s. Nat. Chem. Biol. 11, 611-617 (2015)). PROTAC®s operate by inducing molecular proximity between the protein of interest (POI) and a cellular E3 ligase substrate receptor by binding simultaneously to both proteins. This induced proximity leads to ubiquitination and proteasomal degradation of the POI. Of note, the modular design consisting of a warhead binding to the POI, a flexible linker, and a defined E3 ligase ligand renders PROTAC® development very flexible. The list of proteins permissive to targeted degradation now contains a large number of protein kinases, including one instance of a single-pass transmembrane receptor tyrosine kinase. Some proteins with one (1) transmembrane region, like EGFR, HER2, c-Met, ALK and FLT-3 (Cell Chem Biol. 2018 Jan. 18; 25(1):67-77. The Advantages of Targeted Protein Degradation Over Inhibition: An RTK Case Study. Burslem G M, Smith B E, Lai A C, Jaime-Figueroa S, McQuaid D C, Bondeson D P, Toure M, Dong H, Qian Y, Wang J, Crew A P, Hines J, Crews C M./Eur J Med Chem. 2018 May 10; 151:304-314. Proteolysis Targeting Chimeras (PROTAC®s) of Anaplastic Lymphoma Kinase (ALK). Zhang C, Han X R, Yang X, Jiang B, Liu J, Xiong Y, Jin J. J Am Chem Soc. 2018 Dec. 5; 140(48):16428-16432/Enhancing Antiproliferative Activity and Selectivity of a FLT-3 Inhibitor by Proteolysis Targeting Chimera Conversion. Burslem G M, Song J, Chen X, Hines J, Crews C M) have been shown to be degradable by “PROTAC®” induced degradation.

In one aspect, the present invention relates to compounds of the following formula (I):

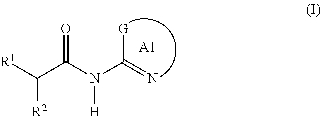

It is to be understood that the present invention also relates to any stereoisomer, tautomer, pharmaceutically acceptable salt, solvate or prodrug of the compounds of formulae (I), (II), (III), (IV) and (V) (and any more specific definitions of the compounds according to the present invention such as formulae (B), (C) etc.). This includes any pharmaceutically acceptable salt of a stereoisomer or tautomer, any solvate of a stereoisomer or tautomer, any solvate of a pharmaceutically acceptable salt, any solvate of a pharmaceutically acceptable salt of a stereoisomer or tautomer, including any prodrugs of any of these.

R¹ is selected from optionally substituted bicyclic aryl and optionally substituted bicyclic heteroaryl. Preferably, R¹ is selected from optionally substituted naphthyl, benzothienyl, benzofuranyl, isobenzofuranyl, chromenyl, indolizinyl, isoindolyl, indolyl (e.g., 3H-indolyl), indazolyl, purinyl, isoquinolyl, quinolyl, phthalazinyl, naphthyridinyl, quinoxalinyl, cinnolinyl, 1,2-benzoisoxazol-3-yl, benzothiazolyl, benzoxazolyl, benzisoxazolyl, benzimidazolyl, coumarinyl, and chromonyl. More preferably, R¹ is selected from optionally substituted naphthyl, benzothienyl, benzofuranyl, indolizinyl, isoindolyl, indolyl (e.g., 3H-indolyl), indazolyl, purinyl, isoquinolyl, quinolyl, phthalazinyl, naphthyridinyl, quinoxalinyl, cinnolinyl, 1,2-benzoisoxazol-3-yl, benzothiazolyl, benzoxazolyl, benzisoxazolyl and benzoimidazolyl. Even more preferably, R¹ is selected from optionally substituted naphthyl, benzothienyl, benzofuranyl, isoquinolyl and quinolyl. Still more preferably, R¹ is selected from optionally substituted naphthyl, benzo[b]thienyl and benzo[b]furanyl. Most preferably, R¹ is selected from optionally substituted naphthyl and benzo[b]furanyl, wherein preferably the optional substituent(s) is/are independently selected from alkyl and heteroalkyl.

It is preferred that R¹ is an optionally substituted bicyclic heteroaryl, such as imidazo-pyridinyl (e.g. imidazo[1,2-a]pyridinyl), pyrrolopyridinyl (e.g. pyrrolo[2,3-b]pyridin-3-yl, pyrrolo[3,2-b]pyridin-3-yl), or quinazolinyl (e.g. (2-oxo)quinazolin-3(4H)-yl).

Examples of such of bicyclic heteroaryls as R¹ include the following, which are optionally substituted

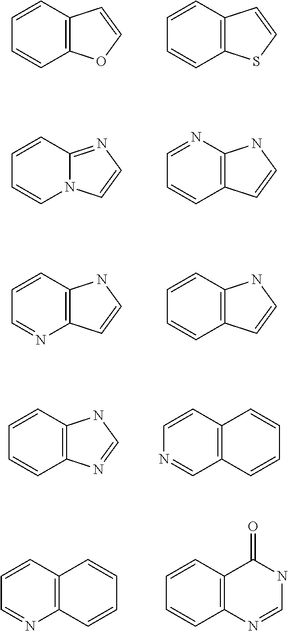

Particular examples of bicyclic heteroaryls as R¹ include the following

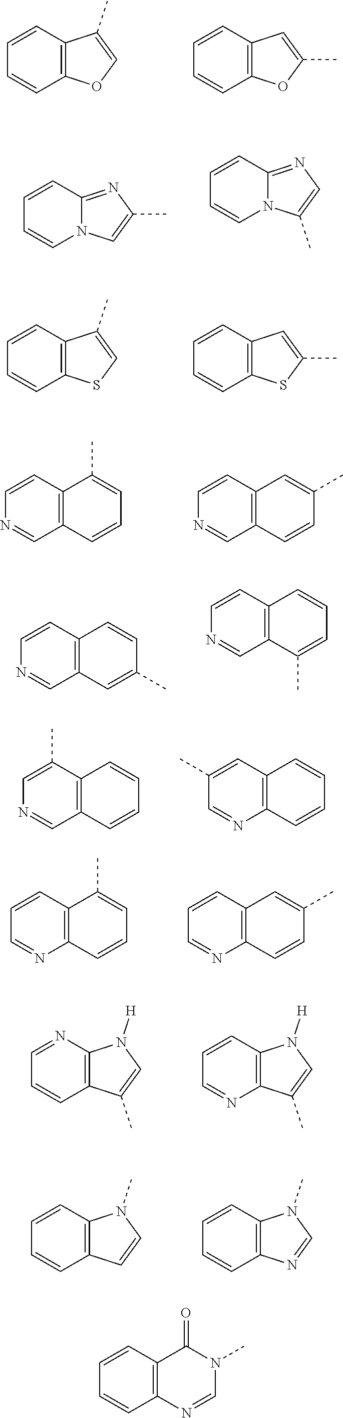

The dashed lines indicate the position at which these bicyclic heteroaryl are attached to the remainder of formula (I). It is to be understood that these preferred examples of the bicyclic heteroaryl of R¹ are optionally substituted as set out herein.

When R¹ is an optionally substituted bicyclic heteroaryl, it is preferred that the optionally substituted bicyclic heteroaryl is linked to the remainder of formula (I) via one of its carbon ring atoms.

Preferred examples of the bicyclic heteroaryl of R¹, are

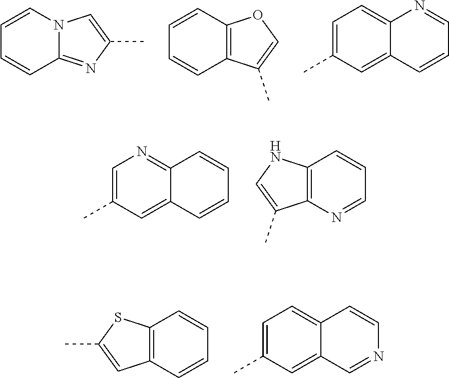

of which the following are more preferred

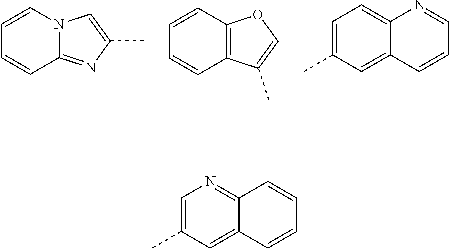

The dashed lines indicate the position at which these bicyclic heteroaryl are attached to the remainder of formula (I). It is to be understood that these preferred examples of the bicyclic heteroaryl of R¹ are optionally substituted as set out in the following.

Particularly preferred examples of R¹ including its optional substituent are:

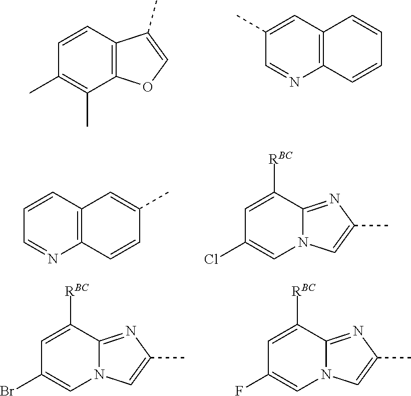

wherein R^{BC} is selected from hydrogen, methyl, methoxy, fluoro, chloro and bromo, preferably from hydrogen, methyl, methoxy, fluoro and chloro.

Even more preferred examples of R¹ including its optional substituent are

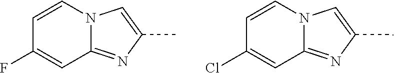

The one or more optional substituent(s) of the optionally substituted bicyclic aryl and optionally substituted bicyclic heteroaryl (including for any specific examples of these) are preferably each independently selected from halogen, alkyl, haloalkyl, haloalkoxy and heteroalkyl. Preferably the one or more optional substituent(s) of the optionally substituted bicyclic aryl and optionally substituted bicyclic heteroaryl (including for any specific examples of these) are preferably each independently selected from halogen, alkyl, haloalkyl and heteroalkyl.

R² is selected from hydrogen and alkyl. Preferably, R² is selected from hydrogen, methyl and ethyl. Even more preferably, R² is selected from hydrogen and methyl. Still more preferably, R² is hydrogen.

A¹ is optionally substituted five- or six-membered monocyclic heteroaryl. Preferably, A¹ is selected from optionally substituted pyrrolyl, furanyl, thienyl, imidazolyl, pyrazolyl, oxazolyl, isoxazolyl, thiazolyl, isothiazolyl, triazolyl, furazanyl, oxadiazolyl, thiadiazolyl, tetrazolyl, pyridinyl, diazinyl (e.g. pyridazinyl, pyrimidinyl, pyrazinyl), oxazinyl, thiazinyl, triazinyl and tetrazinyl. More preferably, A¹ is selected from optionally substituted pyrrolyl, furanyl, thienyl, imidazolyl, pyrazolyl, oxazolyl, isoxazolyl, thiazolyl, isothiazolyl, triazolyl, furazanyl, oxadiazolyl, thiadiazolyl and tetrazolyl. Still more preferably, A¹ is selected from optionally substituted imidazolyl, pyrazolyl, oxazolyl, isoxazolyl, thiazolyl and isothiazolyl. Even more preferably, A¹ is selected from optionally substituted oxazolyl, isoxazolyl, thiazolyl and isothiazolyl. Yet more preferably, A¹ is selected from optionally substituted oxazolyl and thiazolyl.

When G is selected from O, S, NH and N(alkyl), A¹ is preferably selected from optionally substituted imidazolyl, oxazolyl, thiazolyl, triazolyl (in particular 1H-1,2,4-triazolyl and 4H-1,2,4-triazolyl), oxadiazolyl (in particular 1,2,4-oxadiazolyl and 1,3,4-oxadiazolyl), thiadiazolyl (in particular 1,2,4-thiadiazolyl and 1,3,4-thiadiazolyl), tetrazolyl, pyrimidinyl, triazinyl (in particular 1,2,4-triazinyl and 1,3,5-triazinyl) and tetrazinyl (in particular 1,2,4,5-tetrazinyl und 1,2,3,5-tetrazinyl); more preferably from optionally substituted imidazolyl, oxazolyl, thiazolyl, triazolyl (in particular 1H-1,2,4-triazolyl and 4H-1,2,4-triazolyl), oxadiazolyl (in particular 1,2,4-oxadiazolyl and 1,3,4-oxadiazolyl), thiadiazolyl (in particular 1,2,4-thiadiazolyl and 1,3,4-thiadiazolyl) and tetrazolyl; still more preferably from optionally substituted imidazolyl, oxazolyl and thiazolyl; even more preferably from optionally substituted oxazolyl and thiazolyl; and yet more preferably from optionally substituted thiazolyl.

Specific examples of five- or six-membered monocyclic heteroaryl groups of A¹ in formula (I) include

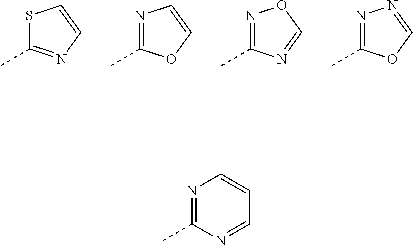

which are each optionally substituted with one or more optional substituent(s) set out in the following. The dashed lines indicate the position at which these five- or six-membered monocyclic heteroaryl are attached to the remainder of formula (I).

The one or more optional substituent(s) of the optionally substituted five- or six-membered monocyclic heteroaryl (including for any specific examples thereof) are preferably each independently selected from halogen, alkyl, haloalkyl, heteroalkyl and cycloalkyl. More preferably, the one or more optional substituent(s) of the optionally substituted five- or six-membered monocyclic heteroaryl (including for any specific examples thereof) are each independently selected from halogen, alkyl, haloalkyl and heteroalkyl. Particularly preferred examples of this substituent are cycloproyl, trifluoromethyl and isopropyl.

Preferred examples of the substituted five- or six-membered monocyclic heteroaryl of A¹, are:

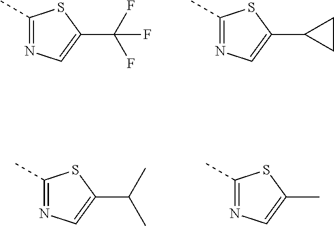

wherein the following are even more preferred

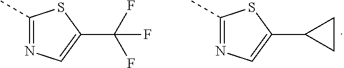

The dashed lines indicate the position at which these five- or six-membered monocyclic heteroaryl are attached to the remainder of formula (I).

G is a ring atom selected from oxygen, sulfur, carbon and nitrogen. Preferably, G is selected from O, S, CH, N, NH and N(alkyl). More preferably, G is selected from O, S, NH and N(alkyl). Still more preferably, G is selected from O, S and NH. Even more preferably, G is selected from O and S. Still even more preferably, G is S.

Preferably, the optional substituent of the optionally substituted five- or six-membered monocyclic heteroaryl may also be present in the position of G. Thus, the optional substituent may in particular also be present at the CH or NH instead of the H.

Specific examples of the compound of formula (I) include the following.

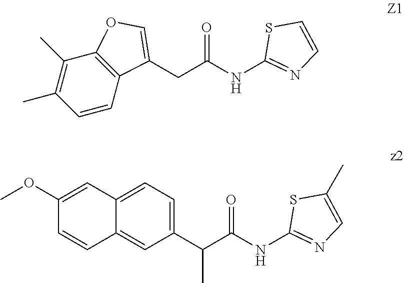

These two formulae are disclaimed from the product claims of the present invention. Optionally, they are also disclaimed from the first and second medical use claims (including methods of treatment) of the present invention.

In alternative embodiments, G in formula (I) is CH wherein H is optionally replaced by one of the optional substituents recited above for the five- or six-membered monocyclic heteroaryl.

Compounds of this type may also be represented by the following formula (B):

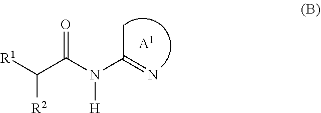

In formula (B), it is to be understood that the upper left corner of ring A¹ represents the CH wherein H is optionally replaced by one of the optional substituents recited above for the five- or six-membered monocyclic heteroaryl. Preferably, the upper left corner of ring A¹ in formula (B) represents CH.

In formula (B), A¹ is preferably selected from optionally substituted pyrrolyl, imidazolyl, pyrazolyl, oxazolyl, isoxazolyl, thiazolyl, isothiazolyl, furazanyl, oxadiazolyl (in particular 1,2,3-oxadiazolyl and 1,2,5-oxadiazolyl), thiadiazolyl (in particular 1,2,3-thiadiazolyl and 1,2,5-thiadiazolyl), pyridinyl, diazinyl (e.g. pyridazinyl, pyrimidinyl, pyrazinyl), triazinyl (in particular 1,2,3-triazinyl and 1,2,4-triazinyl) and tetrazinyl (in particular 1,2,3,4-tetrazinyl). More preferably, A¹ is selected from optionally substituted pyrazolyl and pyridinyl. Even preferably, A¹ is selected from optionally substituted pyrazolyl and pyridinyl. Even more preferably A¹ is optionally substituted pyrazolyl.

R¹ and R² in formula (B) are the same as defined above with respect to formula (I).

Thus, the present invention in particular also relates to compounds of formulae (B-I) and (n-II):

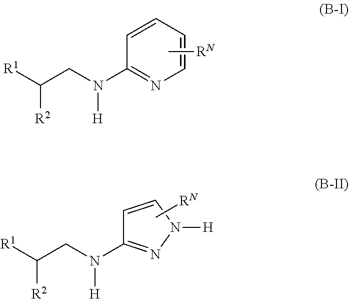

wherein R^{N} indicates the one or more optional substituents of the pyrazolyl and pyridinyl, respectively. It is to be understood that in formula (B-I), the pyridine ring preferably has from 1 to 4, more preferably 1 to 3, even more preferably 1 or 2, most preferably 1 substituents R^{N}. It is furthermore to be understood that in formula (B-II), the pyrazolyl ring preferably has from 1 to 3, more preferably 1 or 2, most preferably 1 substituent(s) R^{N}.

A preferred example of formula (B-I) is the following formula (B-Ia):

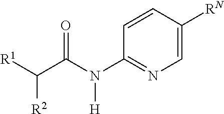

A preferred example of formula (B-II) is the following formula (B-IIa):

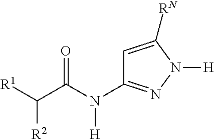

R¹ and R² in formulae (B-I) and (B-II) (as well as any examples thereof) are as defined for formula (I).

The one or more optional substituent(s) of the optionally substituted five- or six-membered monocyclic heteroaryl (including for any specific examples thereof) are preferably each independently selected from halogen, alkyl, haloalkyl, heteroalkyl and cycloalkyl. More preferably, the one or more optional substituent(s) of the optionally substituted five- or six-membered monocyclic heteroaryl (including for any specific examples thereof) are each independently selected from halogen, alkyl, haloalkyl and heteroalkyl. Particularly preferred examples of this substituent are cycloproyl, trifluoromethyl and isopropyl.

The R^{N} is/are likewise preferably each independently selected from halogen, alkyl, cycloalkyl, haloalkyl and heteroalkyl. More preferably, the R^{N} is/are each independently selected from halogen, alkyl, haloalkyl and heteroalkyl. Particularly preferred examples of R^{N} are cycloproyl, trifluoromethyl and isopropyl.

Preferred examples of compounds of formula (I) include

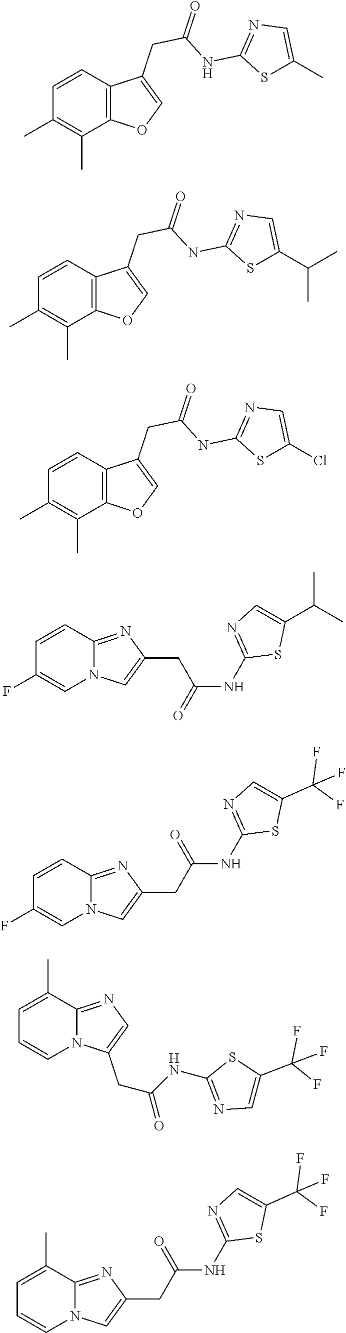

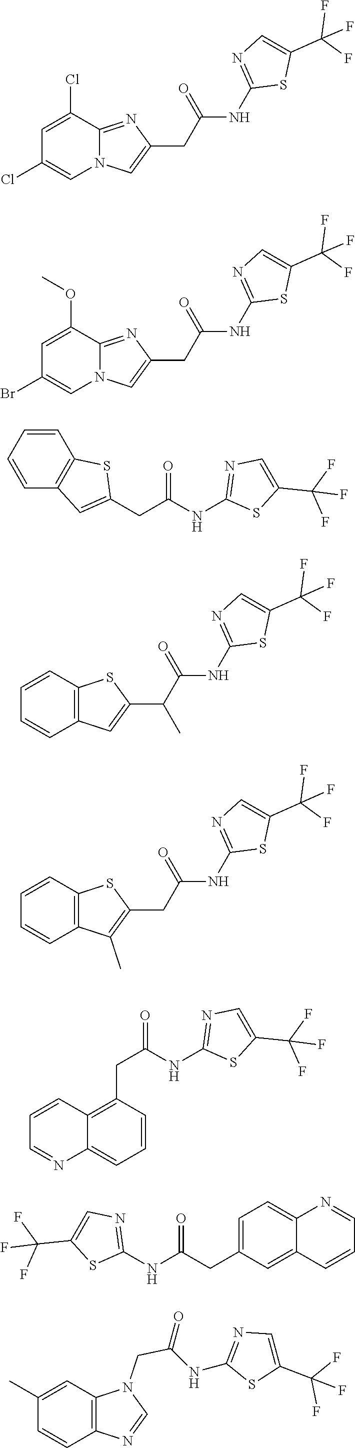

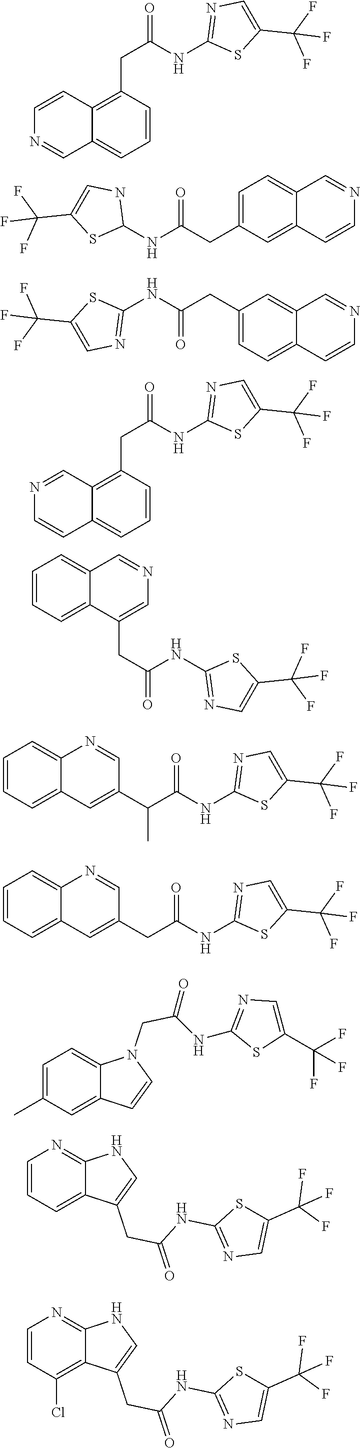

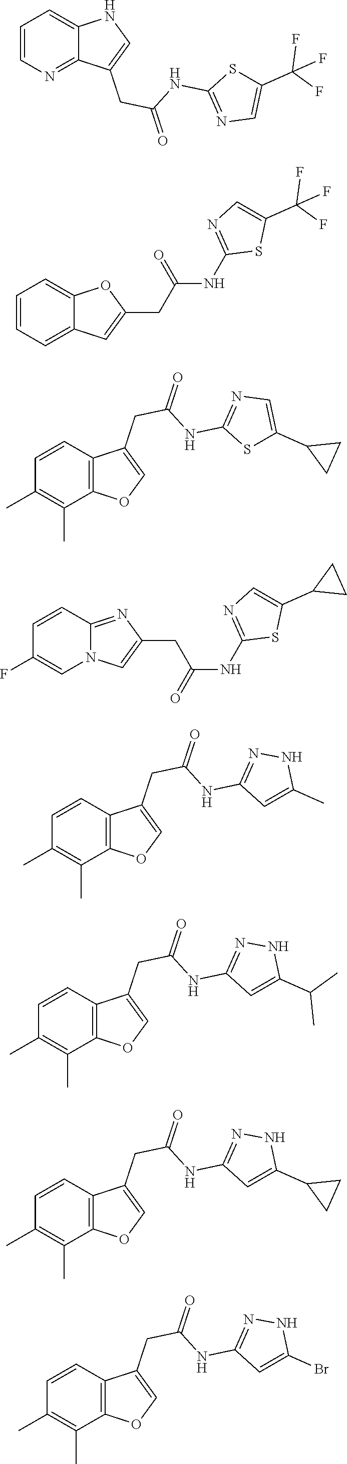

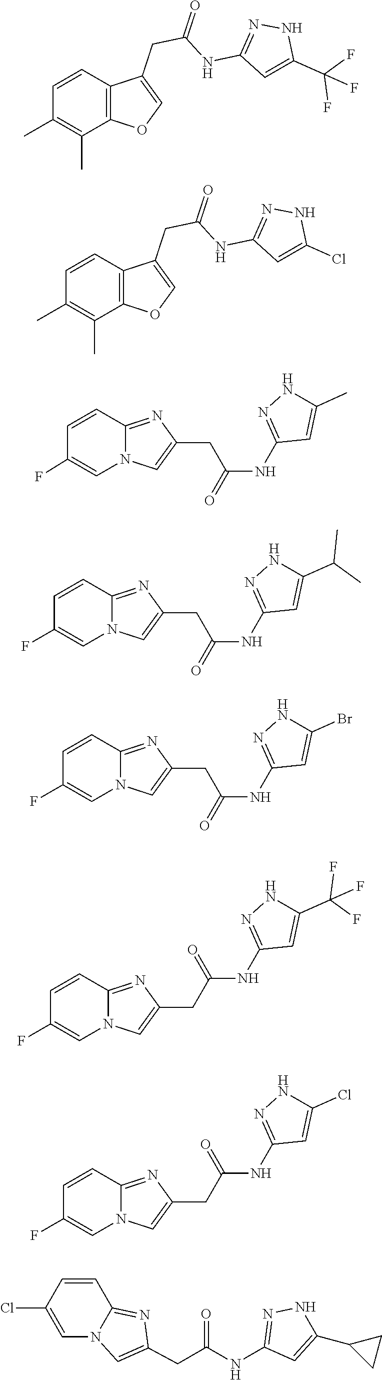

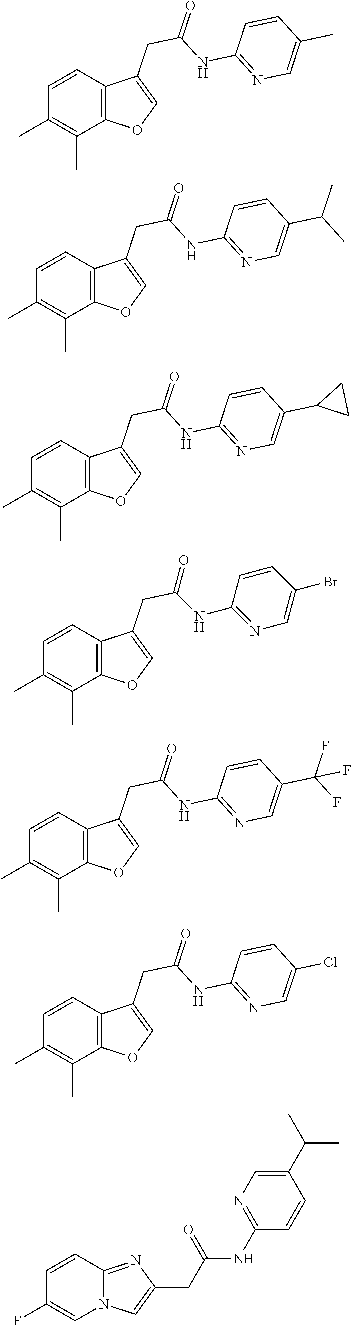

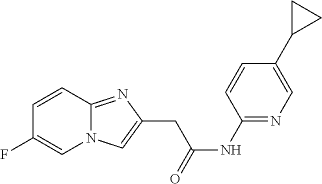 [SMILES: O=C(Cc1cn2cc(F)ccc2n1)Nc1ccc(C2CC2)cn1]

In a further aspect, the present invention relates to compounds of the following formula (C):

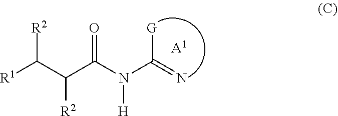

wherein R¹, R², G and A¹ are as defined with respect to formula (I) and it is to be understood that each R² is independently selected from the groups which are given for R² with respect to formula (I). Preferably, both R² are hydrogen.

A particularly preferred example of R¹ for formula (C) is optionally substituted

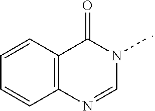

Preferred examples of formula (C) include the following compounds

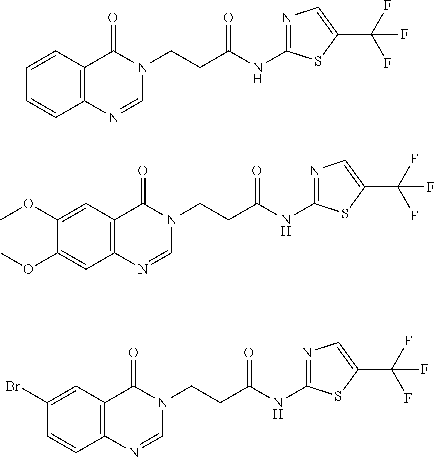 [SMILES: O=C(CCn1cnc2ccccc2c1=O)Nc1ncc(C(F)(F)F)s1 ; COc1cc2ncn(CCC(=O)Nc3ncc(C(F)(F)F)s3)c(=O)c2cc1OC ; O=C(CCn1cnc2ccc(Br)cc2c1=O)Nc1ncc(C(F)(F)F)s1]

In a further aspect, the present invention furthermore relates to compounds of the following formula (II):

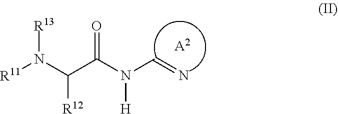

R¹¹ is selected from -(optionally substituted aryl), -(optionally substituted heteroaryl), —C(O)-(optionally substituted aryl) and —C(O)-(optionally substituted heteroaryl). Preferably, R¹¹ is selected from -(optionally substituted aryl), and —C(O)-(optionally substituted aryl). More preferably, R¹¹ is selected from -(optionally substituted phenyl), and —C(O)-(optionally substituted phenyl).

The one or more optional substituent(s) of the -(optionally substituted aryl), -(optionally substituted heteroaryl), —C(O)-(optionally substituted aryl) and —C(O)-(optionally substituted heteroaryl) (including of any specific examples of any of these) are preferably each independently selected from halogen, alkyl, haloalkyl and heteroalkyl.

R¹² is selected from hydrogen and alkyl. R¹³ is selected from hydrogen and alkyl. It is to be understood that R¹² and R¹³ are optionally linked to form, together with the nitrogen and the carbon to which they are connected, an optionally substituted heterocycloalkyl group. This optionally substituted heterocycloalkyl group preferably does not contain any oxygen or sulfur in the ring. It is furthermore preferred that the optionally substituted heterocycloalkyl group contains five or six ring atoms. The one or more optional substituent(s) of the optionally substituted heterocycloalkyl group are preferably selected from halogen, alkyl, haloalkyl and heteroalkyl. The optionally substituted heterocyloalkyl group thus formed is preferably an optionally substituted pyrrolidine group, more preferably a pyrrolidine group without any optional substituents.

It is preferred that either R¹³ is hydrogen and R¹² is methyl, or that R¹² and R¹³ are linked to form, together with the nitrogen and the carbon to which they are connected, an optionally substituted pyrrolidine group.

A² is an optionally substituted five- to ten-membered heteroaryl group. The one or more optional substituent(s) of the optionally substituted five- to ten-membered heteroaryl group (including of any specific examples thereof) are preferably each independently selected from halogen, alkyl, haloalkyl and heteroalkyl. It is preferred that A² is either an optionally substituted five- or six-membered heteroaryl group or an optionally substituted eight- to ten-membered heteroaryl group, wherein the optionally substituted eight- to ten-membered heteroaryl group contains one aromatic ring and one non-aromatic ring. In the optionally substituted eight- to ten-membered heteroaryl group, the aromatic ring is preferably an optionally substituted five- or six-membered heteroaryl group. In the optionally substituted eight- to ten-membered heteroaryl group, the aromatic ring is furthermore preferably the ring containing the nitrogen atom shown as being in Ring A² in formula (II). In other words, in the optionally substituted eight- to ten-membered heteroaryl group, the aromatic ring is preferably the ring which is directly linked to the N—H group shown on the right hand side of formula (II). The optionally substituted five- or six-membered heteroaryl group is preferably selected from pyrrolyl, imidazolyl, pyrazolyl, oxazolyl, isoxazolyl, thiazolyl, isothiazolyl, triazolyl, furazanyl, oxadiazolyl, thiadiazolyl, pyridyl, pyridazinyl, pyrimidinyl, pyrazinyl, and triazinyl. The optionally substituted five- or six-membered heteroaryl group is more preferably selected from imidazolyl, oxazolyl, isoxazolyl, thiazolyl, isothiazolyl, pyridyl, pyridazinyl, pyrimidinyl and pyrazinyl. The optionally substituted five- or six-membered heteroaryl group is even more preferably selected from oxazolyl, thiazolyl, pyridyl, pyridazinyl, pyrimidinyl and pyrazinyl. The optionally substituted five- or six-membered heteroaryl group is still more preferably selected from oxazolyl, thiazolyl and pyridyl. The optionally substituted five- or six-membered heteroaryl group is yet more preferably selected from thiazolyl and pyridyl. These examples also apply to the optionally substituted five- or six-membered heteroaryl group within the optionally substituted eight- to ten-membered heteroaryl group. It is to be understood that the optionally substituted five- or six-membered heteroaryl group is preferably to be arranged such that one of its nitrogen atoms is at the position which is indicated by the nitrogen atom in ring A² in formula (II).

Specific examples of the compounds of formula (II) include the following.

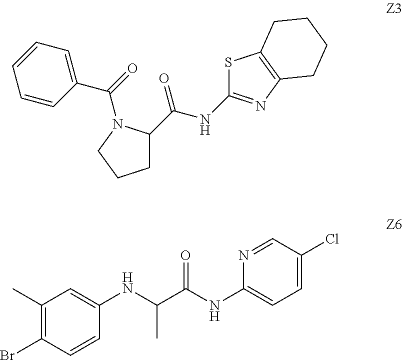 [SMILES: O=C(Nc1nc2c(s1)CCCC2)C1CCCN1C(=O)c1ccccc1 ; Cc1cc(NC(C)C(=O)Nc2ccc(Cl)cn2)ccc1Br]

These two formulae are disclaimed from the product claims of the present invention. Optionally, they are also disclaimed from the first and second medical use claims (including methods of treatment) of the present invention.

In yet a further aspect, the present invention furthermore relates to compounds of the following formula (III):

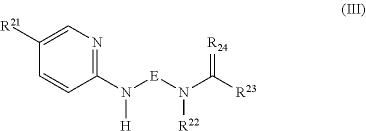

E is a linear C₂₋₄ alkylene group (in particular —(CH₂)₂₋₄—) wherein one or more of the CH₂ units are each optionally replaced by any one independently selected from S, O and NH, wherein the linear C₂₋₄ alkylene group is optionally substituted with 1, 2, 3 or 4 substituents independently selected from ═O, —OH, -Hal, —C₁₋₆ alkyl, and —C₁₋₆ haloalkyl. Preferably, E is a linear C₂₋₃ alkylene group wherein one of the CH₂ units is optionally replaced by one independently selected from S, O and NH, wherein the linear C₂₋₃ alkylene group is optionally substituted with 1 or 2 substituents independently selected from ═O, —OH, -Hal, —C₁₋₆ alkyl, and —C₁₋₆ haloalkyl. More preferably, E is a linear C₂₋₃ alkylene group wherein the linear C₂₋₃ alkylene group is optionally substituted with one substituent selected from ═O, —OH, -Hal, —C₁₋₆ alkyl, and —C₁₋₆ haloalkyl. Even more preferably, E is a linear C₂₋₃ alkylene group wherein the linear C₂₋₃ alkylene group is optionally substituted with one substituent selected from ═O, -Hal, -methyl and -ethyl. Still more preferably, E is selected from —(CH₂)₃—, —(CH₂)₂— and —(C═O)—(CH₂)₂—.

R²¹ is selected from -halogen, —NO₂, —C(═O)H, —C(═O)R²⁶, —COOH, —C(═O)OR²⁷, —CF₃ and —CN, wherein R²⁶ and R²⁷ are each independently selected from alkyl and haloalkyl. Preferably, R²¹ is selected from -halogen, —C(═O)R²⁶, —C(═O)OR²⁷, —CF₃ and —CN, wherein R²⁶ and R²⁷ are each independently selected from alkyl and haloalkyl. Even more preferably, R²¹ is selected from -halogen, —CF₃ and —CN. The -halogen is preferably selected from —Cl and —Br, more preferably —Br.

R²² is selected from hydrogen and alkyl. Alternatively, R²² is linked to R²³, as described herein.

R²³ is selected from optionally substituted aryl and optionally substituted heteroaryl. The one or more optional substituent(s) of the optionally substituted aryl and optionally substituted heteroaryl (including for any specific examples of these) are preferably each independently selected from halogen, alkyl, haloalkyl and heteroalkyl. Preferably, R²³ is optionally substituted aryl, more preferably optionally substituted phenyl. Alternatively, R²³ is linked to R²², as described herein. Alternatively, R²³ is linked to R²⁵, as described herein.

R²⁴ is selected from O and NR²⁵, wherein R²⁵ is selected from alkyl or haloalkyl. Alternatively, R²⁵ is linked to R²³.

When R²² and R²³ are linked, they form, together with the nitrogen and the carbon between R²² and R²³, ring α. Thus, in this case, the compound of formula (III) can be represented by the compound of formula (IIIa):

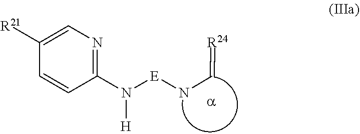

In this formula (IIIa), it is preferred that R²⁴ be O.

Ring α is an optionally substituted 5 or 6-membered heterocyclic group which is optionally anellated. Ring α can thus be an optionally substituted monocylic or bicyclic heterocyclic group. The optionally substituted monocylic or bicyclic heterocyclic group can be saturated, partially unsaturated or aromatic. Either one or (if a contains two rings) both rings can be aromatic, or one ring may be aromatic while the other ring may be partially unsaturated. It is preferred that ring α has a bicyclic structure.

Further preferably, ring α is an optionally substituted pyrimidone or benzpyrimidone. It is to be understood that, in these case, the —N—C(═O)— of the pyrimidine moiety is arranged such that it corresponds to the —N—C(═R²⁴)— moiety shown in formula (IIIa).

The one or more optional substituent(s) of ring α (including of any more specific definitions thereof) are preferably each independently selected from halogen, alkyl, haloalkyl and heteroalkyl.

When R²⁴ is NR²⁵, R²³ and R²⁵ are optionally linked to form, together with the nitrogen and the carbon between the R²³ and the R²⁵ ring β. Thus, in this case, the compound of formula (III) can be represented by the compound of formula (IIIb):

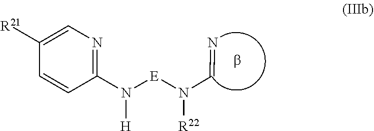

Ring β is an optionally substituted 5 or 6-membered heterocyclic group which is optionally anellated. Ring β can thus be an optionally substituted monocylic or bicyclic heterocyclic group. The optionally substituted monocylic or bicyclic heterocyclic group can be saturated, partially unsaturated or aromatic. Either one or (if R contains two rings) both rings can be aromatic, or one ring may be aromatic while the other ring may be partially unsaturated. It is preferred that ring β has a bicyclic structure.

Further preferably, ring β is an optionally substituted bicyclic aromatic heterocycle containing one or more heteroatoms selected from N, O and S. It is particularly preferred that ring β contains an oxadiazole or a thiadiazole. It is further preferred that ring β is an optionally substituted imidazothiadiazole, more preferably an optionally substituted imidazo[2,1-b][1,3,4]thiadiazole. It is to be understood that the imidazothiadiazole is preferably bound to the nitrogen bearing the R²² substitutent in formula (IIIb) via the non-bridgehead-carbon of the thiadiazole of the imidazothiadiazole.

The one or more optional substituent(s) of ring β (including of any more specific definitions thereof) are preferably each independently selected from halogen, alkyl, haloalkyl and heteroalkyl.

The 5 or 6-membered heterocyclic group preferably refers to a 5 or 6-membered monocyclic ring comprising one or more (e.g., one, two or three) ring heteroatoms independently selected from O, S and N, wherein one or more S ring atoms (if present) and/or one or more N ring atoms (if present) are optionally oxidized, and wherein one or more carbon ring atoms are optionally oxidized.

The term “which is optionally anellated” refers to a state in which two adjacent non-hydrogen atoms of the 5 or 6-membered heterocyclic group are not only part of the 5 or 6-membered heterocyclic group but are also part of another ring which contains in total 5 or 6 ring atoms. The other ring may be carbocyclic or heterocyclic, as well as being saturated, partially unsaturated or aromatic.

Specific examples of the compounds of formula (III) include the following.

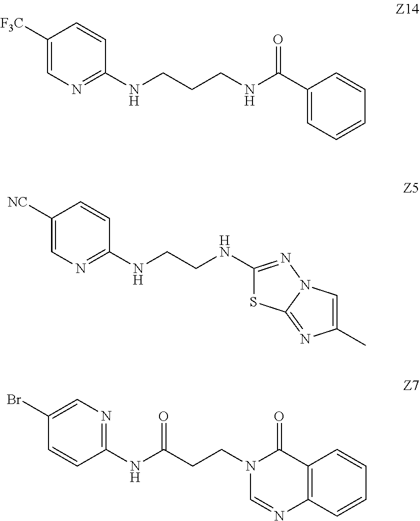 [SMILES: O=C(NCCCNc1ccc(C(F)(F)F)cn1)c1ccccc1 ; Cc1cn2nc(NCCNc3ccc(C#N)cn3)sc2n1 ; O=C(CCn1cnc2ccccc2c1=O)Nc1ccc(Br)cn1]

These three formulae are disclaimed from the product claims of the present invention. Optionally, they are also disclaimed from the first and second medical use claims (including methods of treatment) of the present invention.

In still a further aspect, the present invention furthermore relates to compounds of the following formulae (IV) and (V):

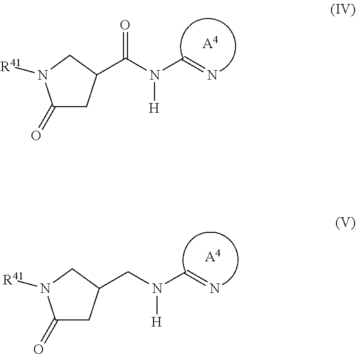

R⁴¹ is selected from -(optionally substituted aryl), -(optionally substituted heteroaryl), -(optionally substituted alkylene)-(optionally substituted aryl) and -(optionally substituted alkylene)-(optionally substituted heteroaryl). Preferably, R⁴¹ is selected from -(optionally substituted aryl) and -(optionally substituted alkylene)-(optionally substituted aryl). More preferably, R⁴¹ is selected from -(optionally substituted aryl), wherein aryl is preferably phenyl.

Among formulae (IV) and (V), formula (IV) is more preferred.

Preferred examples of R⁴¹ include the following

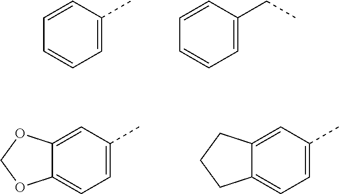

among which the following are preferred

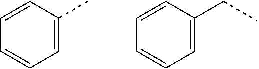

The dashed lines indicate the position at which these groups are attached to the remainder of formula (IV) or (V). It is to be understood that these preferred examples are optionally substituted as set out in the following.

The one or more optional substituent(s) of the optionally substituted aryl, optionally substituted heteroaryl and optionally substituted alkylene (including of any specific examples of any of these) are preferably each independently selected from halogen, alkyl, haloalkyl, heteroalkyl and cycloalkyl. More preferably, the one or more optional substituent(s) of the optionally substituted aryl, optionally substituted heteroaryl and optionally substituted alkylene (including of any specific examples of any of these) are each independently selected from halogen, alkyl, haloalkyl and heteroalkyl.

Preferred examples of R⁴¹ including the optional substituent are

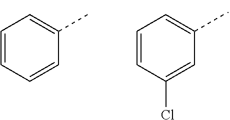

A⁴ is optionally substituted monocyclic or bicyclic heteroaryl. It is to be understood that the optionally substituted monocyclic or bicyclic heteroaryl contains one ring or two fused rings. In the case of two fused rings, one or both (preferably both) rings are aromatic. In particular, A⁴ is optionally substituted five- or six-membered heteroaryl which is optionally anellated. Preferred examples of A⁴ include optionally substituted pyrrolyl (e.g., 2H-pyrrolyl), imidazolyl, pyrazolyl, pyridyl (in particular 2 pyridyl), pyrazinyl, pyrimidinyl, pyridazinyl, indolizinyl, isoindolyl, indolyl (e.g., 3H-indolyl), indazolyl, isoquinolyl, quinolyl, thiazolyl, isothiazolyl, phenothiazinyl, oxazolyl, isoxazolyl, furazanyl, benzothiazolyl, benzoxazolyl, benzisoxazolyl and benzoimidazolyl. More preferred examples of A⁴ include optionally substituted pyridyl, pyrimidinyl, thiazole and benzimidazole.

More preferably, A⁴ is selected from optionally substituted thiazolyl, pyrazolyl and pyridinyl, preferably optionally substituted thiazolyl and pyrazolyl.

Examples of A⁴ including the optional substituent include

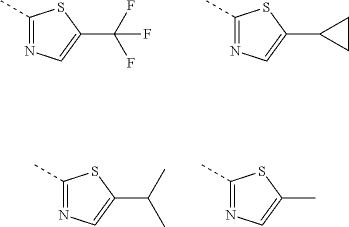

of which the following are more preferred

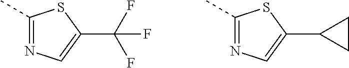

and the following is most preferred

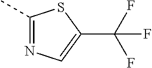

The one or more optional substituent(s) of A⁴ (and of any specific examples of this group), are preferably each independently selected from halogen, alkyl, haloalkyl and heteroalkyl.

Most preferred examples of the compounds of formulae (IV) and (V) include the following.

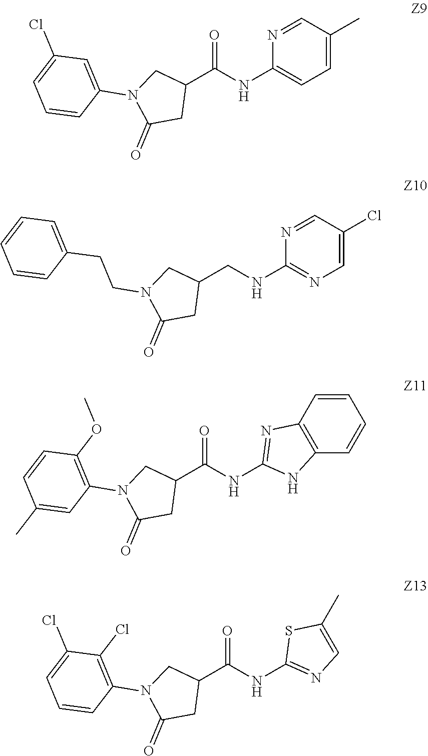

These four formulae are disclaimed from the product claims of the present invention. Optionally, they are also disclaimed from the first and second medical use claims (including methods of treatment) of the present invention.

Preferred examples of the compounds of formula (IV) include the following compounds:

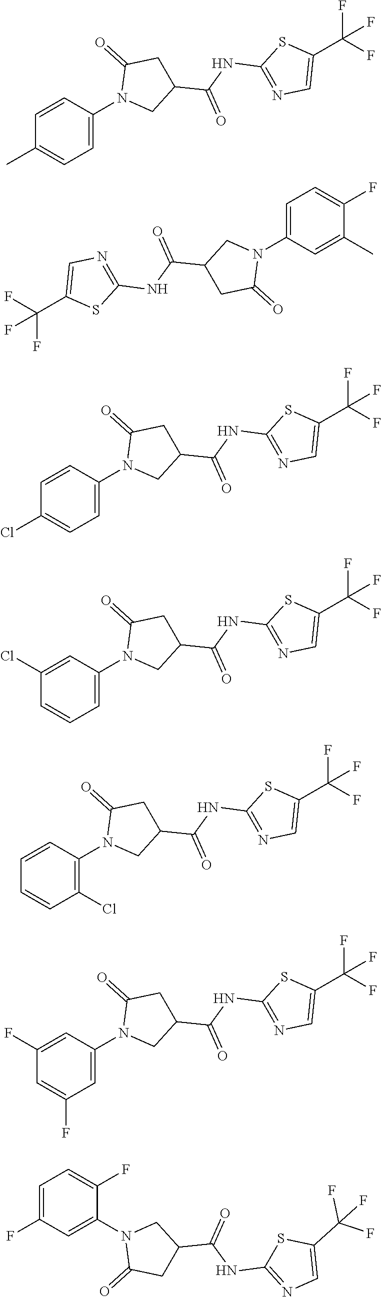

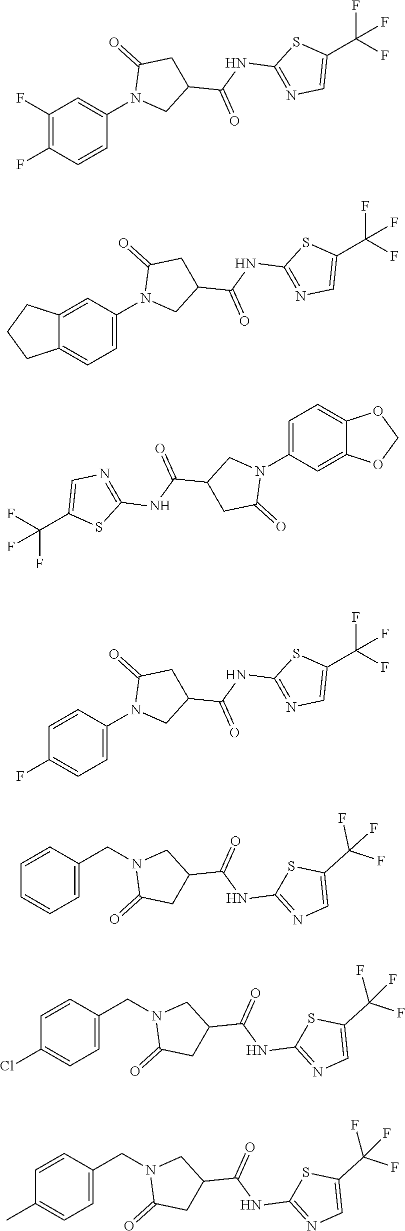

It is to be understood that any reference to a “compound of the present invention” is to be understood as being a reference to any one of the compounds of formulae (I), (II), (III), (IV) and (V).

It is furthermore to be understood that compounds indicated as being disclaimed are optionally also disclaimed in the form of any of their tautomers, pharmaceutically acceptable salts, and solvates. Preferably however, only the compounds as shown in the respective formulae are being disclaimed.

Accordingly, and disclosed herein, the compound may modify the function of the E3 ligase complex/cullin-RING ubiquitin ligase complex/CRL complex. This may occur for example by modifying posttranslational changes of a target protein as outlined above. The modified function of the E3 ligase complex/cullin-RING ubiquitin ligase complex/CRL complex comprises an enhanced activity of the E3 ligase complex/cullin-RING ubiquitin ligase complex/CRL complex. This enhanced activity of the E3 ligase complex/cullin-RING ubiquitin ligase complex/CRL complex may be determined by methods described herein above and herein below and as illustrated in the appended examples. As disclosed herein and as illustrated in the appended Examples, said enhanced activity of the E3 ligase complex/cullin-RING ubiquitin ligase complex/CRL complex may be determined by the measurement of the level/amount of target protein/target proteins in a cell expressing the target protein/target proteins in the presence of the compound and wherein the CRL activity is decreased in said cell compared to a control cell. Said control cell is preferably of the same cell type as the cell wherein the CRL activity is decreased. In the context of this invention, said control cell is also designated as “wild-type cell”.

The terms “E3 ligase binding moiety” and “EBM” or are used interchangeably and means that the E3 ligase binding moiety/EBM is moiety modifying the function of the E3 ligase and/or binding to at least one regulator or member of the E3 ligase complex/cullin-RING ubiquitin ligase complex/CRL complex. “Modifying the function of the E3 ligase” as used in context of the invention means that the cullin-RING ubiquitin ligase activity/CRL activity is enhanced by the E3 ligase binding moiety/EBM, for example by binding of the E3 ligase binding moiety/EBM to the E3 ligase/cullin-RING ubiquitin ligase/CRL or by modifying the function of the E3 ligase complex/cullin-RING ubiquitin ligase complex/CRL complex.

The E3 ligase binding moiety/EBM may bind to or modify the function of the at least one member or regulator of the E3 ligase complex/cullin-RING ubiquitin ligase complex/CRL complex. Such at least one member of the E3 ligase complex/cullin-RING ubiquitin ligase complex/CRL complex may be CUL4B (NP_001073341.1); DDB1(NP_001914.3); RBX1(NP_055063.1); UBE2G1(NP_003333.1); and CUL4A (NP_001008895.1 and all isoforms). For example, at least one member of the E3 ligase complex/cullin-RING ubiquitin ligase complex/CRL complex may be DDB1 (NP_001914.3).

Such at least one regulator of the E3 ligase complex/cullin-RING ubiquitin ligase complex/CRL complex may be UBE2M (NP_003960.1); UBA3 (NP_003959.3); UBE2F(NP_542409.1); NAE1(NP_003896.1);COPS1(NP_001308018.1), COPS2(NP_004227.1),COPS3(NP_003644.2),COPS4(NP_057213.2),COPS5(NP_006828.2), COPS6(NP_006824.2),COPS7A(NP_001157566),COPS7B(NP_073567.1),COPS8(NP_0067 01.1); DCUN1D1(NP_065691.2); DCUN1D2(NP_001014305.1); DCUN1D3(NP_775746.1); DCUN1D4(NP_001035492.1) and DCUN1D5(NP_115675.1). Such at least one member of the E3 ligase complex as disclosed herein and in context of the invention may be identified by their respective accession numbers and/or sequences as provided, for example, by NCBI. Particularly, such at least one member of the E3 ligase complex/cullin-RING ubiquitin ligase complex/CRL complex may be CUL4B or DDB1. More particular, such at least one member of the E3 ligase complex/cullin-RING ubiquitin ligase complex/CRL complex may bind to compounds of the present invention.

Binding of the E3 ligase binding moiety/EBM may to the E3 ligase complex/cullin-RING ubiquitin ligase complex/CRL complex, such as at least one member or regulator of said E3 ligase complex/cullin-RING ubiquitin ligase complex/CRL complex may be determined by methods known in the art. Further methods of how to determine Binding of the E3 ligase binding moiety/EBM may to the E3 ligase complex/cullin-RING ubiquitin ligase complex/CRL complex, such as at least one member or regulator of said E3 ligase complex/cullin-RING ubiquitin ligase complex/CRL complex are known in the art as outlined below. For example, means and methods known in the art of how to determine the E3 ligase binding moiety/EBM may to the E3 ligase complex/cullin-RING ubiquitin ligase complex/CRL complex comprise, inter alia, immunoassays (like Western blots, ELISA tests and the like) and/or reporter assay (like luciferase assays and the like).

In context of the invention, target proteins may include but are not limited to proteins associated with cancer, metabolic disorders, neurologic disorders or infectious diseases.

Non-limiting examples of the such target protein/target proteins associated with cancer may be transcription factors such as ESR1 (NP_000116.2), AR (NP_000035.2), MYB (NP_001123645.1), MYC (NP_002458.2); RNA binding proteins; scaffolding proteins; GTPases such as HRAS (NP_005334.1), NRAS (NP_002515.1), KRAS(NP_203524.1); solute carriers; kinases such as CDK4 (NP_000066.1), CDK6 (NP_001138778.1), CDK9 (NP_001252.1), EGFR (NP_005219.2), SRC (NP_938033.1), PDGFR (NP_002600.1), ABL1 (NP_005148.2), HER2 (NP_004439.2), HER3 (NP_001973.2), BCR-ABL (NP_009297.2), MEK1 (NP_002746.1), ARAF (NP_001645.1), BRAF (NP_004324.2), CRAF (NP_001341618.1), phosphatases, bromodomain- and chromodomain containing proteins such as BRD2 (NP_001106653.1), BRD3 (NP_031397.1), BRD4 (NP_490597.1), CBP (NP_004371.2), p300 (NP_001420.2), ATAD2 (NP_054828.2), SMARCA2 (NP_003061.3), SMARCA4 (NP_001122316.1), PBRM1 (NP_060783.3), G-protein coupled receptors; anti-apoptotic proteins like BCL2 (NP_000624.2) and MCL1 (NP_068779.1), phosphatases such as SHP2 (NP_002825.3), PTPN1 (NP_002818.1), PTPN12 (NP_002826.3); immune regulators such as PDL1 (NP_054862.1) and combinations thereof. Particular non-limiting examples of such target protein/target proteins associated with cancer may be BRD2, BRD3, BRD4, CBP, p300, ATAD2, SMARCA2, SMARCA4, PBRM1, CDK4, CDK6, CDK9, CDK12 (NP_057591.2) and/or CDK13 (NP_003709.3), EWS-FLI (NP_002009.1), CDC6 (NP_001245.1), CENPE (NP_001804.2), EGFR, SRC, PDGFR, ABL1, HER2, HER3, BCR-ABL1, MEK1, ARAF, BRAF, CRAF, HRAS, NRAS, KRAS, BCL2, MCL1, SHP2, PTPN1, PTPN12, ESR1, AR, MYB, MYC, PDL1 and combinations thereof. More particular non-limiting examples of such target protein/target proteins associated with cancer may be KRAS, NRAS, MYC, MYB, ESR1, AR, EGFR, HER2, BCR-ABL and BRAF, even more particular KRAS, NRAS, MYC and MYB. Even more particular non-limiting examples of the one or more target protein/target proteins associated with cancer may be CDK12, CDK13 and/or CCNK, particularly CCNK.

Non-limiting examples of the one or more target protein/target proteins associated with metabolic disorders may be ARX (NP_620689.1), SUR (NP_001274103.1), DPP4 (NP_001926.2) and SGLT (NP_001243243.1). Non-limiting examples of the one or more target protein/target proteins associated with neurologic disorders may be Tau (NP_058519.3) and beta-amyloid (NP_000475.1). Non-limiting examples of the one or more target protein/target proteins associated with infectious diseases may be CCR5 (NP_000570.1) and PLA2G16 (NP_001121675.1).

The term “hypomorphic mutation” refers to a mutation resulting in a reduction-in-function of the at least one member or regulator of an E3 ubiquitin ligase complex. In other words, the hypomorphic mutation results in a decreased activity of the E3 ubiquitin ligase complex compared to the activity of a E3 ubiqutin ligase complex in a corresponding wild-type cell. This decreased activity of the E3 ubiquitin ligase complex may be achieved by a lower expression level and/or level of activity of the at least one member of the E3 ubiquitin ligase complex relative to levels in the corresponding wild-type cell. For example, such a hypomorphic state can be caused by methods known in the art and described herein below. Non-limiting examples of hypomorphic mutations of the at least one member or regulator of the E3 ubiquitin ligase complex include, but are not limited to, Cas9/CRISPR, inhibitors, antibodies, monobodies and nanobodies, nucleic acid molecules including such as RNA and DNA for example antisense oligonucleotides, siRNA, shRNA or miRNA, or any combinations thereof. Accordingly, the inactivation of the at least one member or regulator of the E3 ubiquitin ligase complex refers to a complete or substantially complete loss of function of the at least one member of the E3 ubiquitin ligase complex, which results in decreased CRL activity. Means and methods of how to inactivate the at least one member or regulator of the E3 ubiquitin ligase complex are known in the art may be caused by a mutation in the at least one member or regulator of the E3 ubiquitin ligase complex or can be caused by various synthetic or natural agents or materials that can inhibit the at least one member or regulator of the E3 ubiquitin ligase complex. Such synthetic or natural agents or materials may include but are not limited to small molecules, proteins including antibodies and polypeptides, and nucleic acid molecules including such as RNA and DNA for example antisense oligonucleotides, siRNA, shRNA or miRNA, or any combinations thereof. For example, an inactivation by a mutation in the at least one member or regulator of the E3 ubiquitin ligase complex may be caused by a knock out. Such a knock out may be performed by Cas9/CRISPR (Clustered Regularly interspaced Short Palindromic Repeats). Particularly, a KBM-7 cell comprises a mutated UBE2M, wherein an 18 bp depletion of the UBE2M sequence has been introduced leading to a loss of 16 amino acids (SEQ ID NO.2: AGAC - - - - - - - - - - - - - - - - - - - GTTGGGGTGATAG). A comprising a wild-type UBE2M sequence (SEQ ID NO.1: AGACGTTGCCCTCGAGGTCAATGTTGGGGTGATAG) is used as a control in the appended examples.

As provided herein, such at least one member of the E3 ubiquitin ligase complex which is mutated in the cell by hypomorphic mutation or inactivation resulting in decreased CRL activity may be CUL4B, DDB1, RBX1; UBE2G1 and CUL4A. Accordingly, such at least one regulator of the E3 ubiquitin ligase complex which is mutated in the cell by hypomorphic mutation or inactivation resulting in decreased CRL activity may be UBE2M, UBA3, UBE2F, NAE; COPS1, COPS2, COPS3, COPS5, COPS6, COPS7A, COPS7B, COPS8, DCUN1D2, DCUN1D3, DCUN1D4 and DCUN1D5. Particular examples of such at least one member or regulator of the E3 ubiquitin ligase may be CUL4B or DDB1.

In one embodiment, the compound preferably comprises a moiety binding to at least one member or regulator of the E3 ligase complex. For example, the at least one member or regulator of the E3 ligase complex to which the compound binds may be a substrate receptor, an adaptor protein or a cullin scaffold protein of the E3 ligase complex. Non-limiting examples of such a substrate receptor may be DCAF15, DCAF16, DCAF1, DCAF5, DCAF8, DET1, FBXO7, FBXO22, KDM2A, or KDM2B, particularly CRBN and DCAF15. Non-limiting examples of such an adaptor protein may be DDBL. Non-limiting examples of a such a cullin may be a cullin of the CRL4 complex, such as CUL4A and CUL4B. Thus, for example, a compound as disclosed herein and used in context of the invention comprises a moiety binding to at least one member of the E3 ligase complex, wherein the at least one member of the E3 ligase complex to which the compound binds may be an adaptor protein such as DDB1.

Cullins may be found covalently conjugated with an ubiquitin-like molecule, NEDD8 (neural-precursor-cell-expressed developmentally down-regulated 8). As used herein, the term “NEDD8” refer to a protein that in humans is encoded by the NEDD8 gene. Nucleotide and amino acid sequences of NEDD8 proteins are known in the art. Non-limiting examples of NEDD8 sequences include Homo sapiens NEDD8, the nucleotide and amino acid sequences of which are set forth in GenBank Ace. Nos. NM_006156 and NP_006147, respectively; Mus musculus NEDD8, the nucleotide and amino acid sequences of which are set forth in GenBank Acc. Nos. NM_008683 and NP_032709, respectively (Kamitani et al. (1997) J Biol Chem 272:28557-28562; Kumar et al. (1992) Biochem Biophys Res Comm 185:1155-1161); and Saccharomyces cerevisiae Rub 1, the nucleotide and amino acid sequences of which are set forth in GenBank Acc. Nos. Y16890 and CAA76516, respectively.

The compound of the present invention may bind a target protein/target proteins and bind or modify the function of the E3 ligase complex/cullin-RING ubiquitin ligase complex/CRL complex, for example by recruiting the target protein/target proteins to the E3 ligase complex/cullin-RING ubiquitin ligase complex/CRL complex. For example, the compound may bind to at least one member of the E3 ligase complex/cullin-RING ubiquitin ligase complex/CRL complex and the target protein. As another example, the compound in context of the invention may alter the function of a target protein, for example by modifying posttranslational changes of a target protein. A posttranslational modification may include but is not limited to the phosphorylation status of a protein, e.g. a tyrosine kinase phosphorylating a protein. Thus, the compound may induce ubiquitination of a target protein, e.g., by modifying a target protein in that the target protein becomes accessible for a E3 ligase complex/cullin-RING ubiquitin ligase complex/CRL complex, thereby the compound may not associate with a target protein and/or E3 ligase complex/cullin-RING ubiquitin ligase complex/CRL complex. Non-limiting examples of one or more protein(s) associated with cancer whose degradation may be induced by the compounds of the present invention include DNA-binding proteins including transcription factors such as ESR1, AR, MYB, MYC; RNA binding proteins; scaffolding proteins; GTPases such as HRAS, NRAS, KRAS; solute carriers; kinases such as CCNK, CDK4, CDK6, CDK9, EGFR, SRC, PDGFR, ABL1, HER2, HER3, BCR-ABL, MEK1, ARAF, BRAF, CRAF, particularly such as CDK4, CDK6, CDK9, EGFR, SRC, PDGFR, ABL1, HER2, HER3, BCR-ABL, MEK1, ARAF, BRAF, CRAF, phosphatases, bromodomain- and chromodomain containing proteins such as BRD2, BRD3, BRD4, CBP, p300, ATAD2, SMARCA2, SMARCA4, PBRM1, G-protein coupled receptors; anti-apoptotic proteins such as SHP2, PTPN1, PTPN12; immune regulators such as PDL1 and combinations thereof. Particular non-limiting examples of one or more protein(s) associated with cancer to which the TBM may bind include CDK13, CDK12, CDK9, CDK6, CDK4, CCNK, BRD2, BRD3, BRD4, CBP, p300, ATAD2, SMARCA2, SMARCA4, PBRM1, CDK4, CDK6, CDK9, EWS-FLI, CDC6, CENPE, EGFR, SRC, PDGFR, ABL1, HER2, HER3, BCR-ABL, MEK1, ARAF, BRAF, CRAF, HRAS, NRAS, KRAS, BCL2, MCL2, SHP2, PTPN1, PTPN12, ESR1, AR, MYB, MYC, PDL1 and combinations thereof. Non-limiting examples of one or more protein(s) associated with cancer whose degradation may be induced by the compounds of the present invention may bind include BRD2, BRD3, BRD4, CBP, p300, ATAD2, SMARCA2, SMARCA4, PBRM1, CDK4, CDK6, CDK9, CDK12 and/or CDK13, EWS-FLI, CDC6, CENPE, EGFR, SRC, PDGFR, ABL1, HER2, HER3, BCR-ABL, MEK1, ARAF, BRAF, CRAF, HRAS, NRAS, KRAS, BCL2, MCL2, SHP2, PTPN1, PTPN12, ESR1, AR, MYB, MYC, PDL1 and combinations thereof. More particular non-limiting examples of one or more protein(s) associated with cancer whose degradation may be induced by the compounds of the present invention include KRAS, NRAS, MYC, MYB, ESR1, AR, EGFR, HER2, BCR-ABL and BRAF. Even more particular non-limiting examples of one or more protein(s) associated with cancer whose degradation may be induced by the compounds of the present invention may bind include KRAS, NRAS, MYC and MYB. Non-limiting examples of one or more protein(s) associated with metabolic disorders whose degradation may be induced by the compounds of the present invention include ARX, SUR, DPP4 and SGLT. Non-limiting examples of one or more protein(s) associated with neurologic disorders whose degradation may be induced by the compounds of the present invention include Tau and beta-amyloid. Non-limiting examples of one or more protein(s) associated with infectious diseases are selected from the group consisting of CCR5 and PLA2G16.

Means and methods of how to determine the binding of the compound to the at least one member or regulator of the E3 ligase complex and/or binding to the target protein are known in the art, described herein above and herein below. Such means and methods to determine the binding of a compound to the E3 ubiquitin ligase can be determined, for example, by immunoassays as for instance but not limited to radioimmunoassays, chemiluminescence- and fluorescence-immunoassays, Enzyme-linked immunoassays (ELISA), Luminex-based bead arrays, protein microarray assays, assays suitable for point-of-care testing and rapid test formats such as for instance immune-chromatographic strip tests. Suitable immunoassays may be selected from the group of immunoprecipitation, enzyme immunoassay (EIA)), enzyme-linked immunosorbenassays (ELISA), radioimmunoassay (RIA), fluorescent immunoassay, a chemiluminescent assay, an agglutination assay, nephelometric assay, turbidimetric assay, a Western Blot, a competitive immunoassay, a noncompetitive immunoassay, a homogeneous immunoassay a heterogeneous immunoassay, a bioassay and a reporter assay such as a luciferase assay or Luminex® Assays. An immunoassay is a biochemical test that measures the presence or concentration of a macromolecule/polypeptide in a solution through the use of an antibody or immunoglobulin as a binding agent. According to the invention, the antibodies may be monoclonal as well as polyclonal antibodies. Thus, at least one antibody is a monoclonal or polyclonal antibody. In certain aspects, the level of the marker is determined by high performance liquid chromatography (HPLC). In certain aspects, the HPLC can be coupled to an immunoassay. For example, in a sandwich immunoassay, two antibodies are applied. In principle, all labeling techniques which can be applied in assays of said type can be used, such as labeling with radioisotopes, enzymes, fluorescence-, chemoluminescence- or bioluminescence labels and directly optically detectable color labels, such as gold atoms and dye particles.

Further, binding of a compound to the E3 ubiquitin ligase may be detected, for example, in a Western Blot. Western blotting involves application of a protein sample (lysate) onto a polyacrylamide gel, subsequent separation of said complex mixture by electrophoresis, and transferal or “electro-blotting” of separated proteins onto a second matrix, generally a nitrocellulose or polyvinylidene fluoride (PVDF) membrane. Following the transfer, the membrane is “blocked” to prevent nonspecific binding of antibodies to the membrane surface. Many antibody labeling or tagging strategies are known to those skilled in the art. In the simplest protocols, the transferred proteins are incubated or complexed with a primary enzyme-labeled antibody that serves as a probe. After blocking non-specific binding sites a suitable substrate is added to complex with the enzyme, and together they react to form chromogenic, chemiluminescent, or fluorogenic detectable products that allow for visual, chemiluminescence, or fluorescence detection, respectively. This procedure is described by Gordon et al., U.S. Pat. No. 4,452,901 issued Jun. 15, 1984.

The invention further relates to a method for identifying a compound having the ability to degrade one or more protein(s), the method comprising contacting a compound with a wild-type cell and with a mutated cell, wherein the mutation comprises a hypomorphic mutation or inactivation of at least one member or regulator of an E3 ubiquitin ligase complex; wherein the compound is determined to degrade one or more protein(s) if the level of the one or more protein(s) of the wild-type cell is decreased compared to the mutant cell.

The term “cullin RING ubiquitin E3 ligase” or “CRL” are used interchangeably and refer to an ubiquitin ligase in a complex in which the catalytic core consists of a member of the cullin family and a RING domain protein; the core is associated with one or more additional proteins that confer substrate specificity. The RING domain proteins of the CRL mediate the transfer of ubiquitin from the E2 to the E3-bound substrate. In particular, the cullin RING ubiquitin E3 ligase (CRL) are modular multi-subunit complexes that all contain a common core comprising a cullin subunit and a zinc-binding RING domain subunit. In particular, the cullin subunit folds into an extended structure that forms the backbone of CRLs. The C-terminal region of the cullin subunit forms a globular domain that wraps itself around the RING protein, which in turn recruits the E2 conjugating enzyme to form the enzymatic core. The N-terminal region of the cullin subunit, which resides at the opposite end of the elongated cullin structure, recruits substrate receptors via adapter proteins.

Cullin-based E3 ligases comprise a large family of ubiquitin ligases and are composed of several subunits, consisting of one of seven mammalian cullin homologs (CUL1, CUL2, CUL3, CUL4A/B, CUL5 or CUL7) that bind to the RING domain protein. The cullin N terminus mediates binding of cullin homolog-specific substrate recognition subunits. Binding of the substrate recognition subunits often but not always requires specific adaptor proteins that bridge the interaction with the cullin homologs. For instance, CUL1 is known to bind substrate recognition subunits containing a conserved F-box via the adaptor protein Skp1, thus forming SCF (Skp1-Cul1-F-box) E3 ligases, whereas CUL2 and CUL5 recruit substrate recognition subunits with a VHL or SOCS box, respectively, via the adaptor proteins Elongin B and C. In contrast, CUL3 is known to bind directly to substrate recognition subunits via their BTB domain (also known as POZ domain). CUL4A acts as an assembly factor that provides a scaffold for assembly of a RING-box domain protein (RBX1) and the adaptor protein Damaged DNA Binding Protein 1 (DDB1) (Angers et al., Nature, 2006. 443(7111):590-3). RBX1 is the docking site for the activated E2 protein, and DDB1 recruits substrate specificity receptors or DCAFs (DDB1-cullin4-associated-factors) to form the substrate-presenting side of the CUL4 complex (Angers et al., Nature, 2006. 443(7111):590-3; He et al., Genes Dev, 2006. 20(21):2949-54; Higa et al. Nat Cell Biol, 2006. 8(11): p. 1277-83). Cereblon (CRBN) interacts with damaged DNA binding protein 1 and forms an E3 ubiquitin ligase complex with CUL4 where it functions as a substrate receptor in which the proteins recognized by CRBN might be ubiquitinated and degraded by proteasomes. Cullins may be found covalently conjugated with an ubiquitin-like molecule, NEDD8 (neural-precursor-cell-expressed developmentally down-regulated 8). As used herein, the term “NEDD8” refer to a protein that in humans is encoded by the NEDD8 gene. Nucleotide and amino acid sequences of NEDD8 proteins are known in the art. Non-limiting examples of NEDD8 sequences include Homo sapiens NEDD8, the nucleotide and amino acid sequences of which are set forth in GenBank Ace. Nos. NM_006156 and NP_006147, respectively; Mus musculus NEDD8, the nucleotide and amino acid sequences of which are set forth in GenBank Acc. Nos. NM_008683 and NP_032709, respectively (Kamitani et al. (1997) J Biol Chem 272:28557-28562; Kumar et al. (1992) Biochem Biophys Res Comm 185:1155-1161); and Saccharomyces cerevisiae Rub1, the nucleotide and amino acid sequences of which are set forth in GenBank Acc. Nos. Y16890 and CAA76516, respectively. CRLs may be activated when CRLs are present in a neddylated state, i.e. upon neddylation. As used herein, the term “neddylation” refers to a type of protein modification process by which the ubiquitin-like protein NEDD8 is conjugated to the CRL through E1 activating enzyme (NAE; a heterodimer of NAE1 and UBA3 subunit), E2 conjugating enzyme (Ubc12, UBE2M) and E3 ligase (Gong et al. J. Biol. Chem. 2013; 274: 1203612042). This modification, termed neddylation, activates the E3 ligase activity of CRLs by promoting substrate ubiquitination. The neddylation system is similar to UPS (ubiquitin-proteasome system) in which ubiquitin activating enzyme E1, ubiquitin conjugating enzyme E2 (UBC) and ubiquitin-protein isopeptide ligase E3 are involved (Hershko, A. Cell Death Differ. 2005; 12: 1191-1197). Thus, as used herein, the terms “NAE” or “NEDD8 activating enzyme,” refer to a protein capable of catalyzing the transfer of NEDDS's C terminus to the catalytic cysteine of NEDD8 E2, forming a thiolester-linked E2-NEDD8 intermediate (Gong and Yeh (1999) J Biol Chem 274:12036-12042; and Liakopoulos et al. (1998) EMBO J 17:2208-2214; Osaka et al. (1998) Genes Dev 12:2263-2268). NEDD8 E1 enzymes described in the art include a heterodimer of NAE1 (also referred to as APPBP1; amyloid beta precursor protein binding protein 1; and NEDD8-activating enzyme E1 regulatory subunit). Nucleotide and amino acid sequences of NAE1 proteins are known in the art. Non-limiting examples of NAE1 sequences include Homo sapiens NAE1, the nucleotide and amino acid sequences of which are set forth in GenBank Ace. Nos. NM_001018159 and NP_001018169, respectively; and Mus musculus NAE1, the nucleotide and amino acid sequences of which are set forth in GenBank Ace, Nos. NM_144931 and NP_659180, respectively. NEDD8 E2 enzymes play central roles in the E1-E2-E3 NEDD8 conjugation cascade. As used herein, the terms “NEDD8 conjugating enzyme,” and “NEDD8 E2 enzyme” refer to a protein capable of transiently binding a NEDD8 E1 enzyme for generation and interacting with a NEDD8 E3 ligase. The two known NEDD8 conjugating enzymes are UBC12, which is also known as UBE2M, and UBE2F. Nucleotide and amino acid sequences of UBE2M proteins are known in the art. Non-limiting examples of UBE2M sequences include Homo sapiens UBE2M, the nucleotide and amino acid sequences of which are set forth in GenBank Acc. Nos. NM_003969 and NP_003960, respectively; Mus musculus UBC12, the nucleotide and amino acid sequences of which are set forth in GenBank Ace. Nos. NM_145578 and NP_663553, respectively; and Saccharomyces cerevisiae UBC12, the nucleotide and amino acid sequences of which are set forth in GenBank Acc. Nos. NM_001182194 and NP_013409, respectively.

Neddylation may be reversed by the COP9 signalosome (CSN), which enzymatically removes NEDD8 from a cullin molecule. Thus, the CSN is a central component of the activation and remodeling cycle of cullin-RING E3 ubiquitin ligases (Schlierf et al., Nat. Commun. 7, 13166 (2016)). The human CSN consists of nine protein subunits (COPS1-7A, 7B,8), of which COPS5 contains a metalloprotease motif that provides the catalytic centre to the complex COPS5 exhibits proper deneddylating activity only in the context of the holocomplex and only the fully assembled CSN is competent to specifically remove NEDD8 from CRLs.

The cullin-RING ubiquitin ligase, its activity and means and methods for the detection and/or measurement of this activity may be determined by methods known in the art. For example, such methods may include, but are not limited to FRET (Forster Resonance Energy Transfer) analysis. The theory of FRET (Forster Resonance Energy Transfer) defines a distance dependent, non-radiative transfer of energy from an excited donor (D) to an acceptor molecule (A). The relationship between easily accessible spectroscopic data and theoretical equations was the achievement of Theodor Forster, thereby enabling the possibility of many FRET applications in all kinds of natural sciences. FRET has been used in biochemical applications within the 1 to 10 nm scale (K. E. Sapsford et al., Angew. Chem. Int. Ed., 45, 4562, 2006) (e.g. protein-protein binding, protein folding, molecular interactions at and in cell membranes, DNA hybridization and sequencing, immunoreactions of antigens and antibodies). Details of the theory of FRET are well known. Further examples include protein complementation assay (PCA). Protein complementation assays (PCA) provide a means to detect the interaction of two biomolecules, e.g., polypeptides. PCA utilizes two fragments of the same protein, e.g., enzyme, that when brought into close proximity with each other can reconstitute into a functional, active protein. The NANOBIT® technology (Promega Corporation) may be used to detect molecular proximity by virtue of the reconstitution of a luminescent enzyme via the binding interaction of enzyme components or subunits. By design, the NanoBiT subunits (i.e., 1.3 kDa peptide, 18 kDa polypeptide) weakly associate so that their assembly into a luminescent complex is dictated by the interaction characteristics of the target proteins, such as the at least one member of the E3 ligase complex used herein, onto which they are appended. Details are described, inter alia, in Dixon et al., “NanoLuc Complementation Reporter Optimized for Accurate Measurement of Protein Interactions in Cells,” ACS Chem. Biol., Publication Date (Web): Nov. 16, 2015. In some aspects, the Nano-Glo® HiBiT Detection System (Promega Corporation) may be used to quantify HiBiT-tagged proteins in cell lysates using a add-mix-read assay protocol. Alternatively, HiBiT-tagged proteins, such as ligase substrate receptors, e.g. DCAF15, may be ectopically expressed. HiBit-DCAF15 fusion protein may be ectopically expressed via a viral vector. HiBiT is an 11-amino-acid peptide tag that is fused to the N or C terminus of the protein of interest or inserted into an accessible location within the protein structure. The amount of a HiBiT-tagged protein expressed in a cell may be determined by adding a lytic detection reagent containing the substrate furimazine and Large BiT (LgBiT), the large subunit used in NanoLuc® Binary Technology (NanoBiT®; 1). Alternatively, when the LgBit may be ectopically introduced, such as by but not limited to lentiviral expression, the HiBit level may be measured in living cells by adding luciferase substrate(s).

The term “cancer cell” as used herein means a tumor cell having an ability to proliferate depending on a particular oncogene expressed in the cancer cell. The cancer cell may include a primary cultured cell, a cell line, or a cancer stem cell. As used herein, the “dependence (depending)” concerning the proliferation of the cell refers to the state of the oncogene addiction or the addiction, where the cell proliferates depending on the particular oncogene. Whether or not the cell proliferates depending on the particular oncogene can be confirmed by treating the cell with an inhibitor of the particular oncogene and then evaluating a proliferation ability of the treated cell. For example, the cell as used in context of the method of the invention may be a cancer cell. Particularly, such as cancer cell may be a KBM-7, a Mv4-11 or a Jurkat cell; a pancreatic cancer cell, particularly a AsPC-1 cell; a lung cancer cell, particularly a NCI-H446 cell; a gastric cancer cell; a melanoma cell; a sarcoma cell; a colon cell, particularly a HCT116 or RKO cell; or a neuroblastoma cell, particularly a Be(2)C cell; more particularly the cancer cell may be a KBM-7 cell.

The proliferation ability can be evaluated by, for example, an MTT assay or an MTS assay. It is known that cell death due to apoptosis can be induced, when the cell in the oncogene addiction for the particular oncogene is treated with the inhibitor of such an oncogene. Therefore, the oncogene addiction in the cell for the particular oncogene may be confirmed by evaluating whether or not the apoptosis can be induced by inhibition of the oncogene. The induction of the apoptosis can be evaluated by, for example, a TUNEL assay, detection of active caspase, or detection of annexin V. The cancer cell can be derived from any tissues. Examples of such a tissue may include respiratory tissues (e.g., lung, trachea, bronchi, pharynx, nasal cavity, paranasal cavity), gastrointestinal tissues (e.g., stomach, small intestine, large intestine, rectum), pancreas, kidney, liver, thymus, spleen, heart, thyroid, adrenal, prostate, ovary, uterus, brain, skin, and a blood tissue (e.g., bone marrow, peripheral blood). In another viewpoint, the cancer cell can be an adherent cell or a non-adherent cell (i.e., a blood cell). In still another viewpoint, the cancer cell can be a cell present in the above tissues or tissues other than the above tissues. Examples of such a cell may include a gland cell (e.g., gland cell (adenocyte) in lung, mammary gland cell), an epithelial cell, an endothelial cell, an epidermal cell, an interstitial cell, a fibroblast, an adipocyte, a pancreatic P cell, a nerve cell, a glia cell, and a blood cell.

As used herein, the cancer cell comprises a hypomorphic mutation or inactivation of at least one member of the E3 ligase complex. Thus, a hypomorphic mutation or inactivation of at least one member of the E3 ligase complex may be induced in a cancer cell, e.g., a host cancer cell. The terms “host cell”, “host cell line”, and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. The transformed cell includes transiently or stably transformed cell. Progeny may not be completely identical in nucleic acid content to a parent cell, but may contain mutations. Mutant progeny that have the same function or biological activity as screened or selected for in the originally transformed cell are included herein. In some aspects, the host cell is transiently transfected with the exogenous nucleic acid. In another aspects, the host cell is stably transfected with the exogenous nucleic acid. An “isolated” fusion protein is one that has been separated from the environment of a host cell that recombinantly produces the fusion protein. In some aspects, the fusion protein of the present invention is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of purity, see, e.g., Flatman et al., J. Chromatogr. B 848:79-87 (2007).

As evident from the appended examples, the method for identifying a compound able to induce degradation of one or more protein(s) associated with cancer as provided herein comprises the determination of the viability of a cancer cell compared to a mutant cancer cell, wherein the mutation of said mutant cell comprises a hypomorphic mutation or inactivation of at least one member of the E3 ligase complex. As provided in one aspect of the present invention, the mutated at least one member of the E3 ligase complex results in an impaired activity of the E3 ligase complex, e.g. impaired neddylation of the E3 ligase complex due to the mutation of the at least one member of the E3 ligase complex.

As provided herein, the at least one member of the E3 ligase complex refers to any protein that may be associated, directly or indirectly, with the E3 ligase complex. As used herein, the “at least one member of the E3 ligase complex” refers to a polypeptide comprising an amino acid of which the skilled person in the art is aware of. For examples, the at least one member of the E3 ligase complex as used in accordance with the method of the present invention is at least one member which is in molecular proximity of the CRL, is able to be ubiquitinated by the CRL and is degradable by the CRL.

For example, the mutated at least one member of the E3 ligase complex may be UBE2M. As another example, the mutated at least one member of the E3 ligase complex may be a cullin of the E3 ligase complex, such as CUL4B. As still another example, the mutated at least one member of the E3 ligase complex may be an adaptor protein of the E3 ligase complex, such as DDB1. As yet still another example, the mutated at least one member of the E3 ligase complex may be a substrate receptor, such as cereblon (CRBN) or DCAF15. The term “cereblon” refers to polypeptides (“polypeptides”, “peptides” and “proteins” are used interchangeably herein) comprising the amino acid sequence any CRBN, such as a human CRBN protein (e.g., human CRBN isoform 1, GenBank Accession No. NP-057386; or human CRBN isoforms 2, GenBank Accession No. NP-001166953, each of which is herein incorporated by reference in its entirety), and related polypeptides, including SNP variants thereof. Related CRBN polypeptides include allelic variants (e.g., SNP variants); splice variants; fragments; derivatives; substitution, deletion, and insertion variants; fusion polypeptides; and interspecies homologs, which, in certain aspects, retain CRBN activity and/or are sufficient to generate an anti-CRBN immune response. In another example, the substrate receptor may be DCAF15.

The person skilled in the art knows that also proteins implicated in the pathway of E3 ligase ubiquitination are encompassed by the mutated at least one member of the E3 ligase complex of the invention as long as they result in an impairment in the activity of the E3 ligase complex.

In this context, the skilled person is able to identify such proteins implicated in the E3 ligase ubiquitination pathway. These proteins include but are not limited to, e.g. NAE1. It can also be understood that at least one member of the E3 ligase complex can be inactivated by means other than by mutation, such as by the addition compounds inhibiting at least one member of the E3 ligase complex, such as antibodies or shRNA. Thus, the term inactivation as provided herein also encompasses the use of inhibitory molecules that are able to reduce the activity of the E3 ligase complex by inhibiting at least one member of the E3 ligase complex.

Further, the skilled person is aware of the fact that the CRL activity of a cell may depend on the type of cell used. CRLs may be activated when dissociated from Cullin-associated NEDD8-dissociated protein 1 (CAND1) and/or Cullin-associated NEDD8-dissociated protein 2 (CAND2). The CAND1 gene encodes an essential regulator of Cullin-RING ubiquitin ligases, which are in involved in ubiquitinylation of proteins degraded by the ubiquitin proteasome system. The encoded CAND1 binds to unneddylated cullin-RING box protein complexes and acts as an inhibitor of cullin neddylation and of Skp1, cullin, and F box ubiquitin ligase complex assembly and activity (Liu et al., (2018) Molecular Cell 69, 773-786).

The ubiquitination of these proteins is mediated by a cascade of enzymatic activity. As used herein, “ubiquitin” refers to a polypeptide which is ligated to another polypeptide by ubiquitin ligase enzymes. The ubiquitin can be from any species of organism, preferably a eukaryotic species. Preferably, the ubiquitin is mammalian. More preferably, the ubiquitin is human ubiquitin. In a preferred embodiment, when ubiquitin is ligated to a target protein of interest, that protein is targeted for degradation by the 26S proteasome. Also encompassed by “ubiquitin” are naturally occurring alleles. Ubiquitin is first activated in an ATP-dependent manner by an ubiquitin activating enzyme (E1). The C-terminus of an ubiquitin forms a high energy thiolester bond with E1. The ubiquitin is then passed to an ubiquitin conjugating enzyme (E2; also called ubiquitin carrier protein), also linked to this second enzyme via a thiolester bond. The ubiquitin is finally linked to its target protein to form a terminal isopeptide bond under the guidance of an ubiquitin ligase (E3). In this process, chains of ubiquitin are formed on the target protein, each covalently ligated to the next through the activity of E3. Thus, as used herein, the term “ubiquitination” refers to the covalent attachment of ubiquitin to a protein through the activity of ubiquitination enzymes. E3 enzymes contain two separate activities: an ubiquitin ligase activity to conjugate ubiquitin to target proteins and form ubiquitin chains via isopeptide bonds, and a targeting activity to physically bring the ligase and target protein together. The specificity of the process is controlled by the E3 enzyme, which recognizes and interacts with the target protein to be degraded. Thus, as used herein, the term “ubiquitin ligase”, “ubiquitin E3 ligase” or “E3 ligase” are used interchangeably and refer to an ubiquitination enzyme capable of catalyzing the covalent binding of an ubiquitin to another protein. As used in context of the present invention, it is to be understood that ubiquitination of a target protein such as a protein associated with cancer may be induced if the target protein is in molecular proximity to a CRL. The term “molecular proximity” refers to the physical distance between two molecules that results in a biological event if the molecules are in close proximity to each other. It often but not always involves some chemical bonding, for example non-covalent bonds or covalent bonds.

In one aspect, the present invention relates to a compound for use in medicine. The term “medicine” as used herein is intended to be a generic term inclusive of prescription and non-prescription medications. The compound for use in medicine should be understood as being useful in maintaining health or promoting recovery from a disease, preferably cancer. Further, the term “medicine” includes medicine in any form, including, without limitation, e.g., pills, salves, creams, powders, ointments, capsules, injectable medications, drops, vitamins and suppositories. The scope of this invention is not limited by the type, form or dosage of the medicine. The compounds as described herein and in the context of the present invention, may be for use in treating or preventing cancer, metabolic disorders, neurologic disorders or infectious diseases. In this regard, the compounds as described herein and in the context of the present invention may degrade proteins associated with cancer, metabolic disorders, neurologic disorders or infectious diseases directly or indirectly via the E3 ligase as described herein. For example, proteins associated with cancer, metabolic disorders, neurologic disorders or infectious diseases may be downregulated upon degradation of CCNK by the E3 ligase as shown by the proteomics profiling analysis.

Particularly, proteins associated with neurological disorder such as HECTD1, MBP and FEM1A are downregulated upon degradation of CCNK. As another example, proteins associated with metabolic diseases such as HMMR, LMNA and TMPO are also downregulated upon degradation of CCNK. As still another example, proteins associated with infectious disease such as ICAM2, CALCOCO2 and CDC6 are downregulated upon degradation of CCNK. As yet still another example, cancer associated proteins such as BUB1, BUBIB, MCM10, CDCA7 and CDC6 are also all downregulated upon degradation of CCNK. Thus, proteins that are downregulated upon degradation of CCNK involve proteins associated with cancer, metabolic disorders, neurologic disorders or infectious diseases.

In one aspect of the present invention, the chemical compound or agent is for use in the treatment of cancer. A “disorder,” a “disease,” or a “condition,” as used interchangeably herein, is any condition that would benefit from treatment with a composition (e.g., a pharmaceutical composition) described herein, e.g., a composition (e.g., a pharmaceutical composition) that includes the fusion protein of the present invention. This includes chronic and acute disorders or diseases including those pathological conditions which predispose the mammal to the disorder in question.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the pharmaceutical composition would be administered.

The term “pharmaceutically acceptable”, as used in connection with compositions of the invention, refers to molecular entities and other ingredients of such compositions that are physiologically tolerable and do not typically produce untoward reactions when administered to a mammal (e.g., human). The term “pharmaceutically acceptable” may also mean approved by a regulatory agency of the Federal or a state government or listed in the U.S. Pharmacopeia or other generally recognized pharmacopeia for use in mammals, and more particularly in humans. A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative. Such pharmaceutically acceptable carriers may be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Suitable pharmaceutical carriers are described in “Remington's Pharmaceutical Sciences” by A. R. Gennaro, 20th Edition.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis. “Alleviation,” “alleviating,” or equivalents thereof, refer to both therapeutic treatment and prophylactic or preventative measures, wherein the object is to ameliorate, prevent, slow down (lessen), decrease or inhibit a disease or condition, e.g., the formation of atherosclerotic plaques. Those in need of treatment include those already with the disease or condition as well as those prone to having the disease or condition or those in whom the disease or condition is to be prevented.

The term “cancer” as used herein refers to any malignant tumor in the aforementioned tissue and cell type. Examples of the cancer may include a cancer which can be caused by an abnormal adherent cell, or a cancer which can be caused by an abnormal blood cell (e.g., leukemia, lymphoma, multiple myeloma). Specifically, examples of the cancer which can be caused by the abnormal adherent cell may include a lung cancer (e.g. squamous cell carcinoma, non-small cell carcinoma such as adenocarcinoma and large cell carcinoma, and small cell carcinoma), a gastrointestinal cancer (e.g., stomach cancer, small intestine cancer, large intestine cancer, rectal cancer), a pancreatic cancer, a renal cancer, a hepatic cancer, a thymic cancer, a spleen cancer, a thyroid cancer, an adrenal cancer, a prostate cancer, an urinary bladder cancer, an ovarian cancer, an uterus cancer (e.g., endometrial carcinoma, cervical cancer), a bone cancer, a skin cancer, a brain tumor, a sarcoma, a melanoma, a blastoma (e.g., neuroblastoma), an adenocarcinoma, a planocellular cancer, a solid cancer, an epithelial cancer, and a mesothelioma. Particularly, the cancer may be leukemia, particularly acute myeloid leukemia (AML) and B-cell acute lymphoblastic leukemia (B-ALL) a chronic leukemia, such as chronic myeloid leukemia; adenoid cystic carcinoma; osteosarcoma; ovarian cancer; Ewings sarcoma; lung adenocarcinoma and prostate cancer; lymphoma, neuroblastoma, gastrointestinal cancers, endometrial cancers, medulloblastoma, prostate cancers, esophagus cancer, breast cancer, thyroid cancer, meningioma, liver cancer, colorectal cancer, pancreatic cancer, chondrosarcoma, osteosarcoma, kidney cancer, preferably the cancer is leukemia.

As also discussed above, a cancer to be treated in accordance with the present invention and by the means and methods provided herein may be cancer associated with cell cycle modulators, like cyclin-dependent kinases or transcriptional kinases, like e.g. CDK12, CDK13 and/or cyclins, like CCNK. As used herein a “cancer associated with CDK12, CDK13 and/or CCNK” also includes a cancer associated with a complex of CDK12/13 and CCNK. The same applies, mutatis mutantis, for other disorders discussed herein, like neurological disorders/diseases, matabolic disorders/diseases, and/or infectious diseases. Also these disease may be, in cotext of this invention, associated with cell cycle modulators, like cyclin-dependant kinases or transcriptional kinases, like e.g. CDK12, CDK13 and/or cyclins, like CCNK.

Degradation of CCNK has been described to induce genomic instability of cancer, such as of prostate cancer (see Wu et al 2018, Cell. 2018 Jun. 14; 173(7):1770-1782.e14. doi: 10.1016/j.cell.2018.04.034) and has been suggested to be effective in cancers associated with mutations in DNA damage response genes such as those described in Table 1 of Lord et al 2016, Nat Rev Cancer. 2016 February; 16(2):110-20. doi: 10.1038/nrc.2015.21. Epub 2016 Jan. 18. Further, CCNK degradation has been described to be particularly effective in cancers associated with increased levels of cyclin E1. Thus, as described herein, a cancer associated with cell-cycle modulators, like CDK12, CDK13 and/or CCNK includes, but is not limited, to cancer with an overexpression of cyclin E1 such as breast cancer, ovarian cancer, melanoma, bladder cancer, gastric cancer, stomach adenocarcinoma, lung squamous cancer, lung adenocarcinoma, glioblastoma multiforme and colorectal cancer; see Lei et al.; Nat Commun. 2018 May 14; 9(1):1876.

The “cancer” in cancer-related terms such as terms “cancer cell” and “cancer gene (oncogene)” can also mean the same meaning. The cancer cell can be derived from any mammalian species. Such a mammalian species may include, for example, humans, monkeys, cattle, swines, mice, rats, guinea pigs, hamsters, and rabbits. The mammalian species is preferably the human in terms of clinical application. Therefore, the cancer cell may be a cancer cell isolated from a patient with cancer or a cancer cell derived therefrom. The cancer cell may be a cell not infected with virus or a cell infected with virus. Examples of a carcinogenic virus capable of infecting the cell may include Epstein Barr virus, hepatitis virus, human papilloma virus, human T cell leukemia virus, and Kaposi sarcoma-associated herpes virus. The cancer cell may also be a cancer cell derived from an embryonic stem cell, a somatic stem cell, or an artificial stem cell (e.g., iPS cell) produced from a normal cell. The cancer cell from which the artificial cell of the present invention is derived can express an inherent oncogene. As used herein, the term “inherent oncogene” means an oncogene responsible for proliferation of the cancer cell, which is expressed by the cancer cell that can be used as a material in the establishment of the artificial cell of the present invention. The oncogene can be a gene that is overexpressed in the cancer cell (e.g., overexpression due to increase of copy number of the gene) and transmits a signal for proliferation excessively, or a gene that a mutation occurs which continuously transmit a proliferation signal in the cancer cell. Examples of the mutation may include point mutation (e.g., substitution), deletion, addition, insertion, and mutation causing a fusion (e.g., inversion, translocation). As used herein, the term “gene” may intend to be a mutated gene. Examples of the inherent oncogene may include genes for kinase such as tyrosine kinase (receptor type, and non-receptor type) and serine/threonine kinase, small G-proteins, and transcription factors. Examples of the tyrosine kinase which can play a role in proliferation of the cancer cell may include molecules belonging to an epidermal growth factor receptor (EGFR) family (e.g., EGFR, HER2, HER3, HER4), molecules belonging to platelet derived growth factor receptor (PDGFR) family (e.g., PDGFRα, PDGFRβ), an anaplastic lymphoma kinase (ALK), a hepatocyte growth factor receptor (c-MET), and a stem cell factor receptor (c-KIT). As another example, of kinases which can play a role in proliferation of the cancer may include CDK12, CDK13 and/or CCNK. For example, CDK12, CDK13 and/or CCNK can play a role in proliferation of cancer including but not limited to breast cancer, ovarian cancer, melanoma, bladder cancer, gastric cancer, stomach adenocarcinoma, lung squamous cancer, lung adenocarcinoma, glioblastoma multiforme and colorectal cancer.

In one aspect, the present invention further relates to a method treating cancer comprising administering the chemical compound or agent to a patient having cancer. For example, the compound may be a compound binding to one or more protein(s) to be degraded, wherein the one or more protein(s) are proteins associated with cancer and may be a kinase such as a kinase selected from the group consisting of cyclin-dependent kinases and/or transcriptional kinases, like CDK12, CDK13 and/or cyclins, like CCNK. In this context, the invention may relate to a method for treating cancer comprising administering the chemical compound or agent to a patient having cancer, wherein the compound may be a compound binding to one or more protein(s) selected from the group consisting of CDK12, CDK13 and/or CCNK. For example, said chemical compound or agent is used for the treatment of cancer, wherein said cancer may be selected from breast cancer, ovarian cancer, melanoma, bladder cancer, gastric cancer, stomach adenocarcinoma, lung squamous cancer, lung adenocarcinoma, glioblastoma multiforme and colorectal cancer.

A “patient” or “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the patient, individual, or subject is a human. In one embodiment, the patient may be a “cancer patient,” i.e. one who is suffering or at risk for suffering from one or more symptoms of cancer.

It will be understood that the specific dose level for any particular patient will depend upon a variety of factors including the activity of the specific compound employed, the age, body weight, general health, sex, diet, time of administration, route of administration, rate of excretion, drug combination and the causative mechanism and severity of the particular disease undergoing therapy.

As used herein, the terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, the expression “X is optionally substituted with Y” (or “X may be substituted with Y”) means that X is either substituted with Y or is unsubstituted. Likewise, if a component of a composition is indicated to be “optional”, the invention specifically relates to both possibilities, i.e., that the corresponding component is present (contained in the composition) or that the corresponding component is absent from the composition. It is to be understood that where a list of groups is preceded by the expression “optionally substituted”, the expression “optionally substituted” applies to each one of the respective groups in that list, not just to the first item in the list.

Various groups are referred to as being “optionally substituted” in this specification. Generally, these groups may carry one or more substituents, such as, e.g., one, two, three or four substituents. It will be understood that the maximum number of substituents is limited by the number of attachment sites available on the substituted moiety. Unless defined otherwise, the “optionally substituted” groups referred to in this specification carry preferably not more than two substituents and may, in particular, carry only one substituent. Moreover, unless defined otherwise, it is preferred that the optional substituents are absent, i.e. that the corresponding groups are unsubstituted. If the term “optionally substituted” appears before a list of chemical groups, it is to be understood that it applies to each and every member of this list of chemical groups. Unless explicitly indicted otherwise, the one or more optional substituent(s) are preferably each independently selected from halogen, CN, OH, NH₂, alkyl, haloalkyl, heteroalkyl (preferably alkoxy), haloalkoxy, NH(alkyl), N(alkyl)₂, cycloalkyl, cycloheteroalkyl, monocyclic aryl and monocyclic heteroaryl, wherein the cycloalkyl, cycloheteroalkyl, monocyclic aryl and monocyclic heteroaryl are each independently optionally further substituted with one or more selected from halogen, CN, OH, NH₂, alkyl, haloalkyl, heteroalkyl (including alkoxy), NH(alkyl), and N(alkyl)₂. In cases where two substituents can be present on the same carbon atom, the optional substituent ═O is also encompassed, as it corresponds to a hydrated form of two OH groups on the same carbon atom. Unless explicitly indicted otherwise, the one or more optional substituent(s) are preferably each independently selected from halogen, alkyl, haloalkyl and heteroalkyl.

As used herein, the term “halogen” refers to fluoro (—F), chloro (—Cl), bromo (—Br), or iodo (—I).

As used herein, the term “alkyl” refers to a monovalent saturated acyclic (i.e., non-cyclic) hydrocarbon group which may be linear or branched. Accordingly, an “alkyl” group does not comprise any carbon-to-carbon double bond or any carbon-to-carbon triple bond. The term “alkyl” preferably refers to a “C₁₋₆ alkyl”. A “C₁₋₆ alkyl” denotes an alkyl group having 1 to 6 carbon atoms. Preferred exemplary alkyl groups are methyl, ethyl, propyl (e.g., n-propyl or isopropyl), or butyl (e.g., n-butyl, isobutyl, sec-butyl, or tert-butyl). Unless defined otherwise, the term “alkyl” more preferably refers to C₁₄ alkyl, more preferably to methyl or ethyl, and even more preferably to methyl. As used herein, the term “alkoxy” refers to “—O-alkyl”, wherein “alkyl” is as defined above.

As used herein, the term “haloalkyl” refers to an alkyl group substituted with one or more (preferably 1 to 6, more preferably 1 to 3) halogen atoms which are selected independently from fluoro, chloro, bromo and iodo, and are preferably all fluoro atoms. It will be understood that the maximum number of halogen atoms is limited by the number of available attachment sites and, thus, depends on the number of carbon atoms comprised in the alkyl moiety of the haloalkyl group. “Haloalkyl” may, e.g., refer to —CF₃, —CHF₂, —CH₂F, —CF₂—CH₃, —CH₂—CF₃, —CH₂—CHF₂, —CH₂—CF₂—CH₃, —CH₂—CF₂—CF₃, or —CH(CF₃)₂. As used herein, the term “haloalkoxy” refers to “—O-haloalkyl”, wherein “haloalkyl” is as defined above.

As used herein, the term “heteroalkyl” refers to an alkyl group in which one or two of the —CH₂— groups have been replaced each independently by a group selected from —O—, —S— and —N(C₁₋₆alkyl)-. A preferred example is an alkoxy group such as methoxy.

As used herein, the term “alkenyl” refers to a monovalent unsaturated acyclic hydrocarbon group which may be linear or branched and comprises one or more (e.g., one or two) carbon-to-carbon double bonds while it does not comprise any carbon-to-carbon triple bond. The term “C₂₋₆ alkenyl” denotes an alkenyl group having 2 to 6 carbon atoms. Preferred exemplary alkenyl groups are ethenyl, propenyl (e.g., prop-1-en-1-yl, prop-1-en-2-yl, or prop-2-en-1-yl), butenyl, butadienyl (e.g., buta-1,3-dien-1-yl or buta-1,3-dien-2-yl), pentenyl, or pentadienyl (e.g., isoprenyl). Unless defined otherwise, the term “alkenyl” preferably refers to C₂₋₆ alkenyl, more preferably C₂₋₆ alkenyl.

As used herein, the term “alkynyl” refers to a monovalent unsaturated acyclic hydrocarbon group which may be linear or branched and comprises one or more (e.g., one or two) carbon-to-carbon triple bonds and optionally one or more carbon-to-carbon double bonds. The term “C₂₋₆ alkynyl” denotes an alkynyl group having 2 to 6 carbon atoms. Preferred exemplary alkynyl groups are ethynyl, propynyl, or butynyl. Unless defined otherwise, the term “alkynyl” preferably refers to C₂₋₆ alkynyl, more preferably C₂₋₄ alkynyl.

As used herein, the term “aryl” refers to an aromatic hydrocarbon ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic). “Aryl” may, e.g., refer to phenyl, naphthyl, dialinyl (i.e., 1,2-dihydronaphthyl), tetralinyl (i.e., 1,2,3,4-tetrahydronaphthyl), anthracenyl, or phenanthrenyl. Unless defined otherwise, an “aryl” preferably has 6 to 14 ring atoms, more preferably 6 to 10 ring atoms, and most preferably refers to phenyl. The term “bicyclic aryl” refers to an aromatic hydrocarbon ring group, containing to, preferably anellated, aromatic rings. “Bicyclic aryl” may, e.g., refer to naphthyl. Unless defined otherwise, an “bicyclic aryl” preferably has 10 ring atoms.

As used herein, the term “heteroaryl” refers to an aromatic ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic), wherein said aromatic ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). “Heteroaryl” may, e.g., refer to thienyl (i.e., thiophenyl), benzo[b]thienyl, naphtho[2,3-b]thienyl, thianthrenyl, furyl (i.e., furanyl), benzofuranyl, isobenzofuranyl, chromenyl, xanthenyl, phenoxathiinyl, pyrrolyl (e.g., 2H-pyrrolyl), imidazolyl, pyrazolyl, pyridyl (i.e., pyridinyl; e.g., 2-pyridyl, 3-pyridyl, or 4-pyridyl), pyrazinyl, pyrimidinyl, pyridazinyl, indolizinyl, isoindolyl, indolyl (e.g., 3H-indolyl), indazolyl, purinyl, isoquinolyl, quinolyl, phthalazinyl, naphthyridinyl, quinoxalinyl, cinnolinyl, pteridinyl, carbazolyl, beta-carbolinyl, phenanthridinyl, acridinyl, perimidinyl, phenanthrolinyl (e.g., [1,10]phenanthrolinyl, [1,7]phenanthrolinyl, or [4,7]phenanthrolinyl), phenazinyl, thiazolyl, isothiazolyl, phenothiazinyl, oxazolyl, isoxazolyl, furazanyl, phenoxazinyl, pyrazolo[1,5-a]pyrimidinyl (e.g., pyrazolo[1,5-a]pyrimidin-3-yl), 1,2-benzoisoxazol-3-yl, benzothiazolyl, benzoxazolyl, benzisoxazolyl, benzimidazolyl, 1H-tetrazolyl, 2H-tetrazolyl, coumarinyl, or chromonyl. Unless defined otherwise, a “heteroaryl” preferably refers to a 5 to 14 membered (more preferably 5 to 10 membered) monocyclic ring or fused ring system comprising one or more (e.g., one, two, three or four) ring heteroatoms independently selected from O, S and N, wherein one or more S ring atoms (if present) and/or one or more N ring atoms (if present) are optionally oxidized, and wherein one or more carbon ring atoms are optionally oxidized; even more preferably, a “heteroaryl” refers to a 5 or 6 membered monocyclic ring comprising one or more (e.g., one, two or three) ring heteroatoms independently selected from O, S and N, wherein one or more S ring atoms (if present) and/or one or more N ring atoms (if present) are optionally oxidized, and wherein one or more carbon ring atoms are optionally oxidized. A particularly preferred example of the term “heteroaryl” is pyridiyl. The term “bicyclic heteroaryl” refers to an aromatic ring group, containing two, preferably anellated, rings, wherein one or both rings are aromatic. “Bicyclic heteroaryl” may, e.g., refer to benzo[b]thienyl, benzofuranyl, isobenzofuranyl, chromenyl, indolizinyl, isoindolyl, indolyl (e.g., 3H-indolyl), indazolyl, purinyl, isoquinolyl, quinolyl, phthalazinyl, naphthyridinyl, quinoxalinyl, cinnolinyl, 1,2-benzoisoxazol-3-yl, benzothiazolyl, benzoxazolyl, benzisoxazolyl, benzimidazolyl, coumarinyl, or chromonyl. Unless defined otherwise, a “bicyclic heteroaryl” preferably has 8 to 12 ring atoms, more preferably 9 or 10 ring atoms.

It is to be understood that expressions such as “five or six-membered heterocyclic group” indicate a heterocyclic group having 5 or 6 atoms in the ring. Similarly, expressions such as “five to ten-membered heteroaryl group” indicate a heteroaryl group having 5 to 10 atoms in the one or two rings. Thus, “x-membered” in the context of cyclic groups indicates the number x of ring atoms in the one or more rings but does not imply any limitations as to the number of non-ring atoms, such as hydrogens which are typically present as substituents on the ring(s).

As used herein, the term “cycloalkyl” refers to a saturated hydrocarbon ring group, including monocyclic rings as well as bridged ring, spiro ring and/or fused ring systems (which may be composed, e.g., of two or three rings; such as, e.g., a fused ring system composed of two or three fused rings). “Cycloalkyl” may, e.g., refer to cyclopropyl, cyclobutyl, cyclopentyl, cyclohexyl, cycloheptyl, or adamantyl. Unless defined otherwise, “cycloalkyl” preferably refers to a C₃₁₁ cycloalkyl, and more preferably refers to a C₃₋₈ cycloalkyl. A particularly preferred “cycloalkyl” is a monocyclic saturated hydrocarbon ring having 3 to 8 ring members.

As used herein, the term “cycloheteroalkyl” (which may also be referred to as “heterocycloalkyl”) refers to a saturated ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). “Cycloheteroalkyl” may, e.g., refer to oxetanyl, tetrahydrofuranyl, piperidinyl, piperazinyl, aziridinyl, azetidinyl, pyrrolidinyl, imidazolidinyl, morpholinyl (e.g., morpholin-4-yl), pyrazolidinyl, tetrahydrothienyl, octahydroquinolinyl, octahydroisoquinolinyl, oxazolidinyl, isoxazolidinyl, azepanyl, diazepanyl, oxazepanyl or 2-oxa-5-aza-bicyclo[2.2.1]hept-5-yl. Unless defined otherwise, “cycloheteroalkyl” preferably refers to a 3 to 11 membered saturated ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, and wherein one or more carbon ring atoms are optionally oxidized; more preferably, “cycloheteroalkyl” refers to a 5 to 8 membered saturated monocyclic ring group containing one or more (e.g., one, two, or three) ring heteroatoms independently selected from O, S and N, wherein one or more S ring atoms (if present) and/or one or more N ring atoms (if present) are optionally oxidized, and wherein one or more carbon ring atoms are optionally oxidized.

As used herein, terms such as “binding to at least one member of the E3 ligase complex” do not necessarily imply that the binding has to be directly to a moiety of the E3 ligase. Rather the compound may bind to a protein being part of the E3 ligase complex or a protein which interacts (before or after binding of the compound to the protein, optionally as part of a complex of proteins) with the E3 ligase complex.

A skilled person will appreciate that the substituent groups comprised in the compounds of the present invention (in particular of formulae (I), (II), (III), (IV) and (V)) may be attached to the remainder of the respective compound via a number of different positions of the corresponding specific substituent group. Unless defined otherwise, the preferred attachment positions for the various specific substituent groups are as illustrated in the examples.

As used herein, unless explicitly indicated otherwise or contradicted by context, the terms “a”, “an” and “the” are used interchangeably with “one or more” and “at least one”. Thus, for example, a composition comprising “a” compound of the present invention (in particular of formulae (I), (II), (III), (IV) and (V)) can be interpreted as referring to a composition comprising “one or more” compounds of the present invention.

As used herein, the term “comprising” (or “comprise”, “comprises”, “contain”, “contains”, or “containing”), unless explicitly indicated otherwise or contradicted by context, has the meaning of “containing, inter alia”, i.e., “containing, among further optional elements, . . . ”. In addition thereto, this term also includes the narrower meanings of “consisting essentially of” and “consisting of”. For example, the term “A comprising B and C” has the meaning of “A containing, inter alia, B and C”, wherein A may contain further optional elements (e.g., “A containing B, C and D” would also be encompassed), but this term also includes the meaning of “A consisting essentially of B and C” and the meaning of “A consisting of B and C” (i.e., no other components than B and C are comprised in A).

Moreover, unless indicated otherwise, any reference to an industry standard, a pharmacopeia, or a manufacturer's manual refers to the corresponding latest version that was available at the priority date (i.e., at the earliest filing date) of the present specification.

The scope of the invention embraces all pharmaceutically acceptable salt forms of the compounds provided herein, particularly the compounds of the present invention (in particular of formulae (I), (II), (III), (IV) and (V)), which may be formed, e.g., by protonation of an atom carrying an electron lone pair which is susceptible to protonation, such as an amino group, with an inorganic or organic acid, or as a salt of an acid group (such as a carboxylic acid group) with a physiologically acceptable cation. Exemplary base addition salts comprise, for example: alkali metal salts such as sodium or potassium salts; alkaline earth metal salts such as calcium or magnesium salts; zinc salts; ammonium salts; aliphatic amine salts such as trimethylamine, triethylamine, dicyclohexylamine, ethanolamine, diethanolamine, triethanolamine, procaine salts, meglumine salts, ethylenediamine salts, or choline salts; aralkyl amine salts such as N,N-dibenzylethylenediamine salts, benzathine salts, benethamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts, lysine salts, or histidine salts. Exemplary acid addition salts comprise, for example: mineral acid salts such as hydrochloride, hydrobromide, hydroiodide, sulfate salts (such as, e.g., sulfate or hydrogensulfate salts), nitrate salts, phosphate salts (such as, e.g., phosphate, hydrogenphosphate, or dihydrogenphosphate salts), carbonate salts, hydrogencarbonate salts, perchlorate salts, borate salts, or thiocyanate salts; organic acid salts such as acetate, propionate, butyrate, pentanoate, hexanoate, heptanoate, octanoate, cyclopentanepropionate, decanoate, undecanoate, oleate, stearate, lactate, maleate, oxalate, fumarate, tartrate, malate, citrate, succinate, adipate, gluconate, glycolate, nicotinate, benzoate, salicylate, ascorbate, pamoate (embonate), camphorate, glucoheptanoate, or pivalate salts; sulfonate salts such as methanesulfonate (mesylate), ethanesulfonate (esylate), 2-hydroxyethanesulfonate (isethionate), benzenesulfonate (besylate), p-toluenesulfonate (tosylate), 2-naphthalenesulfonate (napsylate), 3-phenylsulfonate, or camphorsulfonate salts; glycerophosphate salts; and acidic amino acid salts such as aspartate or glutamate salts.

Moreover, the scope of the invention embraces the compounds provided herein, particularly the compounds of the present invention (in particular of formulae (I), (II), (III), (IV) and (V)), in any solvated form, including, e.g., solvates with water (i.e., as a hydrate) or solvates with organic solvents such as, e.g., methanol, ethanol or acetonitrile (i.e., as a methanolate, ethanolate or acetonitrilate), or in any crystalline form (i.e., as any polymorph), or in amorphous form. It is to be understood that such solvates of the compounds provided herein, particularly the compounds of the present invention, also include solvates of pharmaceutically acceptable salts of the corresponding compounds.

Furthermore, the compounds provided herein, particularly the compounds of formulae (I), (II), (III), (IV) and (V), may exist in the form of different isomers, in particular stereoisomers (including, e.g., geometric isomers (or cis/trans isomers), enantiomers and diastereomers) or tautomers. All such isomers of the compounds provided herein are contemplated as being part of the present invention, either in admixture or in pure or substantially pure form. As for stereoisomers, the invention embraces the isolated optical isomers of the compounds according to the invention as well as any mixtures thereof (including, in particular, racemic mixtures/racemates). The racemates can be resolved by physical methods, such as, e.g., fractional crystallization, separation or crystallization of diastereomeric derivatives, or separation by chiral column chromatography. The individual optical isomers can also be obtained from the racemates via salt formation with an optically active acid followed by crystallization. The present invention further encompasses any tautomers of the compounds provided herein.

The scope of the invention also embraces the compounds provided herein, particularly the compounds of formulae (I), (II), (III), (IV) and (V), in which one or more atoms are replaced by a specific isotope of the corresponding atom. For example, the invention encompasses compounds of formulae (I), (II), (III), (IV) and (V), in which one or more hydrogen atoms (or, e.g., all hydrogen atoms) are replaced by deuterium atoms (i.e., ²H; also referred to as “D”). Accordingly, the invention also embraces compounds of formulae (I), (II), (III), (IV) and (V) which are enriched in deuterium. Naturally occurring hydrogen is an isotopic mixture comprising about 99.98 mol-% hydrogen-1 (¹H) and about 0.0156 mol-% deuterium (²H or D).

The content of deuterium in one or more hydrogen positions in the compounds of formulae (I), (II), (III), (IV) and (V) can be increased using deuteration techniques known in the art. For example, a compound of formulae (I), (II), (III), (IV) and (V) or a reactant or precursor to be used in the synthesis of the compound of formulae (I), (II), (III), (IV) and (V) can be subjected to an H/D exchange reaction using, e.g., heavy water (D₂O). Further suitable deuteration techniques are described in: Atzrodt J et al., Bioorg Med Chem, 20(18), 5658-5667, 2012; William J S et al., Journal of Labelled Compounds and Radiopharmaceuticals, 53(11-12), 635-644, 2010; or Modvig A et al., J Org Chem, 79, 5861-5868, 2014. The content of deuterium can be determined, e.g., using mass spectrometry or NMR spectroscopy. Unless specifically indicated otherwise, it is preferred that the compound of formulae (I), (II), (III), (IV) and (V) is not enriched in deuterium. Accordingly, the presence of naturally occurring hydrogen atoms or ¹H hydrogen atoms in the compounds of formulae (I), (II), (III), (IV) and (V) is preferred.

The present invention also embraces the compounds provided herein, particularly the compounds of formulae (I), (II), (III), (IV) and (V), in which one or more atoms are replaced by a positron-emitting isotope of the corresponding atom, such as, e.g., ¹⁸F, ¹¹C, ¹³N, ¹⁵O, ⁷⁶Br, ⁷⁷Br, ¹²⁰I and/or ¹²⁴. Such compounds can be used as tracers or imaging probes in positron emission tomography (PET). The invention thus includes (i) compounds of formulae (I), (II), (III), (IV) and (V), in which one or more fluorine atoms (or, e.g., all fluorine atoms) are replaced by ¹⁸F atoms, (ii) compounds of formulae (I), (II), (III), (IV) and (V), in which one or more carbon atoms (or, e.g., all carbon atoms) are replaced by ¹¹C atoms, (iii) compounds of formulae (I), (II), (III), (IV) and (V), in which one or more nitrogen atoms (or, e.g., all nitrogen atoms) are replaced by ¹³N atoms, (iv) compounds of formulae (I), (II), (III), (IV) and (V), in which one or more oxygen atoms (or, e.g., all oxygen atoms) are replaced by ¹⁵O atoms, (v) compounds of formulae (I), (II), (III), (IV) and (V), in which one or more bromine atoms (or, e.g., all bromine atoms) are replaced by ⁷⁶Br atoms, (vi) compounds of formulae (I), (II), (III), (IV) and (V), in which one or more bromine atoms (or, e.g., all bromine atoms) are replaced by ⁷⁷Br atoms, (vii) compounds of formulae (I), (II), (III), (IV) and (V), in which one or more iodine atoms (or, e.g., all iodine atoms) are replaced by ¹²⁰I atoms, and (viii) compounds of formulae (I), (II), (III), (IV) and (V), in which one or more iodine atoms (or, e.g., all iodine atoms) are replaced by ¹²⁴I atoms. In general, it is preferred that none of the atoms in the compounds of formulae (I), (II), (III), (IV) and (V) are replaced by specific isotopes.

Pharmaceutically acceptable prodrugs of the compounds provided herein, particularly the compounds of formulae (I), (II), (III), (IV) and (V), are derivatives which have chemically or metabolically cleavable groups and become, by solvolysis or under physiological conditions, the compounds of the invention which are pharmaceutically active in vivo. Prodrugs of the compounds according to the the present invention may be formed in a conventional manner with a functional group of the compounds such as, e.g., with an amino, hydroxy or carboxy group. The prodrug form often offers advantages in terms of solubility, tissue compatibility or delayed release in a mammalian organism (see, Bundgaard, H., Design of Prodrugs, pp. 7-9, 21-24, Elsevier, Amsterdam 1985). Prodrugs include acid derivatives, such as, e.g., esters prepared by reaction of the parent acidic compound with a suitable alcohol, or amides prepared by reaction of the parent acid compound with a suitable amine. If a compound of the present invention has a carboxyl group, an ester derivative prepared by reacting the carboxyl group with a suitable alcohol or an amide derivative prepared by reacting the carboxyl group with a suitable amine is exemplified as a prodrug. An especially preferred ester derivative as a prodrug is methylester, ethylester, n-propylester, isopropylester, n-butylester, isobutylester, tert-butylester, morpholinoethylester, N,N-diethylglycolamidoester or α-acetoxyethylester. If a compound of the present invention has a hydroxy group, an acyloxy derivative prepared by reacting the hydroxyl group with a suitable acylhalide or a suitable acid anhydride is exemplified as a prodrug. An especially preferred acyloxy derivative as a prodrug is —OC(═O)—CH₃, —OC(═O)—C₂H₅, —OC(═O)-(tert-Bu), —OC(═O)—C₁₅H₃₁, —OC(═O)-(m-COONa-Ph), —OC(═O)—CH₂CH₂COONa, —O(C═O)—CH(NH₂)CH₃ or —OC(═O)—CH₂—N(CH₃)₂. If a compound of the present invention has an amino group, an amide derivative prepared by reacting the amino group with a suitable acid halide or a suitable mixed anhydride is exemplified as a prodrug. An especially preferred amide derivative as a prodrug is —NHC(═O)—(CH₂)₂OCH₃ or —NHC(═O)—CH(NH₂)CH₃.

The compounds provided herein, including in particular the compounds of formulae (I), (II), (III), (IV) and (V), may be administered as compounds per se or may be formulated as medicaments. The medicaments/pharmaceutical compositions may optionally comprise one or more pharmaceutically acceptable excipients, such as carriers, diluents, fillers, disintegrants, lubricating agents, binders, colorants, pigments, stabilizers, preservatives, antioxidants, and/or solubility enhancers.

The pharmaceutical compositions may comprise one or more solubility enhancers, such as, e.g., poly(ethylene glycol), including poly(ethylene glycol) having a molecular weight in the range of about 200 to about 5,000 Da (e.g., PEG 200, PEG 300, PEG 400, or PEG 600), ethylene glycol, propylene glycol, glycerol, a non-ionic surfactant, tyloxapol, polysorbate 80, macrogol-15-hydroxystearate (e.g., Kolliphor® HS 15, CAS 70142-34-6), a phospholipid, lecithin, dimyristoyl phosphatidylcholine, dipalmitoyl phosphatidylcholine, distearoyl phosphatidylcholine, a cyclodextrin, α-cyclodextrin, β-cyclodextrin, γ-cyclodextrin, hydroxyethyl-β-cyclodextrin, hydroxypropyl-β-cyclodextrin, hydroxyethyl-γ-cyclodextrin, hydroxypropyl-γ-cyclodextrin, dihydroxypropyl-β-cyclodextrin, sulfobutylether-β-cyclodextrin, sulfobutylether-γ-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, a carboxyalkyl thioether, hydroxypropyl methylcellulose, hydroxypropylcellulose, polyvinylpyrrolidone, a vinyl acetate copolymer, vinyl pyrrolidone, sodium lauryl sulfate, dioctyl sodium sulfosuccinate, or any combination thereof.

The pharmaceutical compositions can be formulated by techniques known to the person skilled in the art, such as the techniques published in “Remington: The Science and Practice of Pharmacy”, Pharmaceutical Press, 22^{nd} edition. The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems.

The compounds provided herein, particularly the compounds of formulae (I), (II), (III), (IV) and (V), or the above described pharmaceutical compositions comprising such a compound may be administered to a subject by any convenient route of administration, whether systemically/peripherally or at the site of desired action, including but not limited to one or more of: oral (e.g., as a tablet, capsule, or as an ingestible solution), topical (e.g., transdermal, intranasal, ocular, buccal, and sublingual), parenteral (e.g., using injection techniques or infusion techniques, and including, for example, by injection, e.g., subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, or intrasternal by, e.g., implant of a depot, for example, subcutaneously or intramuscularly), pulmonary (e.g., by inhalation or insufflation therapy using, e.g., an aerosol, e.g., through mouth or nose), gastrointestinal, intrauterine, intraocular, subcutaneous, ophthalmic (including intravitreal or intracameral), rectal, or vaginal administration.

If said compounds or pharmaceutical compositions are administered parenterally, then examples of such administration include one or more of: intravenously, intraarterially, intraperitoneally, intrathecally, intraventricularly, intraurethrally, intrasternally, intracardially, intracranially, intramuscularly or subcutaneously administering the compounds or pharmaceutical compositions, and/or by using infusion techniques. For parenteral administration, the compounds are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Said compounds or pharmaceutical compositions can also be administered orally in the form of tablets, capsules, ovules, elixirs, solutions or suspensions, which may contain flavoring or coloring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include lactose, starch, a cellulose, or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

Alternatively, said compounds or pharmaceutical compositions can be administered in the form of a suppository or pessary, or may be applied topically in the form of a gel, hydrogel, lotion, solution, cream, ointment or dusting powder. The compounds of the present invention may also be dermally or transdermally administered, for example, by the use of a skin patch.

Said compounds or pharmaceutical compositions may also be administered by sustained release systems. Suitable examples of sustained-release compositions include semi-permeable polymer matrices in the form of shaped articles, e.g., films, or microcapsules. Sustained-release matrices include, e.g., polylactides (see, e.g., U.S. Pat. No. 3,773,919), copolymers of L-glutamic acid and gamma-ethyl-L-glutamate (Sidman, U. et al., Biopolymers 22:547-556 (1983)), poly(2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP133988). Sustained-release pharmaceutical compositions also include liposomally entrapped compounds. Liposomes containing a compound of the present invention can be prepared by methods known in the art, such as, e.g., the methods described in any one of: DE3218121; Epstein et al., Proc. Natl. Acad. Sci. (USA) 82:3688-3692 (1985); Hwang et al., Proc. Natl. Acad. Sci. (USA) 77:4030-4034 (1980); EP0052322; EP0036676; EP088046; EP0143949; EP0142641; JP 83-118008; U.S. Pat. Nos. 4,485,045; 4,544,545; and EP0102324.

Said compounds or pharmaceutical compositions may also be administered by the pulmonary route, rectal routes, or the ocular route. For ophthalmic use, they can be formulated as micronized suspensions in isotonic, pH adjusted, sterile saline, or, preferably, as solutions in isotonic, pH adjusted, sterile saline, optionally in combination with a preservative such as a benzalkonium chloride. Alternatively, they may be formulated in an ointment such as petrolatum.

It is also envisaged to prepare dry powder formulations of the compounds provided herein, particularly the compounds of formulae (I), (II), (III), (IV) and (V), for pulmonary administration, particularly inhalation. Such dry powders may be prepared by spray drying under conditions which result in a substantially amorphous glassy or a substantially crystalline bioactive powder. Accordingly, dry powders of the compounds of the present invention can be made according to the emulsification/spray drying process disclosed in WO 99/16419 or WO 01/85136. Spray drying of solution formulations of the compounds of the invention can be carried out, e.g., as described generally in the “Spray Drying Handbook”, 5th ed., K. Masters, John Wiley & Sons, Inc., NY (1991), in WO 97/41833, or in WO 03/053411.

For topical application to the skin, said compounds or pharmaceutical compositions can be formulated as a suitable ointment containing the active compound suspended or dissolved in, for example, a mixture with one or more of the following: mineral oil, liquid petrolatum, white petrolatum, propylene glycol, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, 2-octyldodecanol, benzyl alcohol and water.

The present invention thus relates to the compounds or the pharmaceutical compositions provided herein, wherein the corresponding compound or pharmaceutical composition is to be administered by any one of: an oral route; topical route, including by transdermal, intranasal, ocular, buccal, or sublingual route; parenteral route using injection techniques or infusion techniques, including by subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, intrasternal, intraventricular, intraurethral, or intracranial route; pulmonary route, including by inhalation or insufflation therapy; gastrointestinal route; intrauterine route; intraocular route; subcutaneous route; ophthalmic route, including by intravitreal, or intracameral route; rectal route; or vaginal route. Particularly preferred routes of administration are oral administration or parenteral administration.

Typically, a physician will determine the actual dosage which will be most suitable for an individual subject. The specific dose level and frequency of dosage for any particular individual subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual subject undergoing therapy.

A proposed, yet non-limiting dose of the compounds according to the invention for oral administration to a human (of approximately 70 kg body weight) may be 0.05 to 8000 mg, preferably 0.1 mg to 4000 mg, of the active ingredient per unit dose. The unit dose may be administered, e.g., 1 to 3 times per day. The unit dose may also be administered 1 to 7 times per week, e.g., with not more than one administration per day. A further exemplary dose of the compounds of formulae (I), (II), (III), (IV) and (V) for oral administration to a human is 50 to 200 mg/kg bodyweight/day, particularly 100 mg/kg/day. It will be appreciated that it may be necessary to make routine variations to the dosage depending on the age and weight of the patient/subject as well as the severity of the condition to be treated. The precise dose and also the route of administration will ultimately be at the discretion of the attendant physician or veterinarian.

The compounds provided herein, particularly the compound of formulae (I), (II), (III), (IV) and (V), or a pharmaceutical composition comprising such a compound can be administered in monotherapy (e.g., without concomitantly administering any further therapeutic agents, or without concomitantly administering any further therapeutic agents against the same disease that is to be treated or prevented with the compound of formulae (I), (II), (III), (IV) and (V)). However, the compound of formulae (I), (II), (III), (IV) and (V) or a pharmaceutical composition comprising the compound of formulae (I), (II), (III), (IV) and (V) can also be administered in combination with one or more further therapeutic agents. If the compound of formulae (I), (II), (III), (IV) and (V) is used in combination with a second therapeutic agent active against the same disease or condition, the dose of each compound may differ from that when the corresponding compound is used alone, in particular, a lower dose of each compound may be used. The combination of the compound of formulae (I), (II), (III), (IV) and (V) with one or more further therapeutic agents (such as, e.g., a BRD4 inhibitor, preferably a direct BRD4 inhibitor) may comprise the simultaneous/concomitant administration of the compound of formulae (I), (II), (III), (IV) and (V) and the further therapeutic agent(s) (either in a single pharmaceutical formulation or in separate pharmaceutical formulations), or the sequential/separate administration of the compound of formulae (I), (II), (III), (IV) and (V) and the further therapeutic agent(s). If administration is sequential, either the compound of formulae (I), (II), (III), (IV) and (V) according to the invention or the one or more further therapeutic agents may be administered first. If administration is simultaneous, the one or more further therapeutic agents may be included in the same pharmaceutical formulation as the compound of formulae (I), (II), (III), (IV) and (V), or they may be administered in one or more different (separate) pharmaceutical formulations.

Preferably, the one or more further therapeutic agents to be administered in combination with a compound of the present invention are anticancer drugs. The anticancer drug(s) to be administered in combination with a compound of formulae (I), (II), (III), (IV) and (V) according to the invention may, e.g., be selected from: a tumor angiogenesis inhibitor (e.g., a protease inhibitor, an epidermal growth factor receptor kinase inhibitor, or a vascular endothelial growth factor receptor kinase inhibitor); a cytotoxic drug (e.g., an antimetabolite, such as purine and pyrimidine analog antimetabolites); an antimitotic agent (e.g., a microtubule stabilizing drug or an antimitotic alkaloid); a platinum coordination complex; an anti-tumor antibiotic; an alkylating agent (e.g., a nitrogen mustard or a nitrosourea); an endocrine agent (e.g., an adrenocorticosteroid, an androgen, an anti-androgen, an estrogen, an anti-estrogen, an aromatase inhibitor, a gonadotropin-releasing hormone agonist, or a somatostatin analog); or a compound that targets an enzyme or receptor that is overexpressed and/or otherwise involved in a specific metabolic pathway that is misregulated in the tumor cell (e.g., ATP and GTP phosphodiesterase inhibitors, histone deacetylase inhibitors, protein kinase inhibitors (such as serine, threonine and tyrosine kinase inhibitors, e.g., Abelson protein tyrosine kinase inhibitors) and the various growth factors, their receptors and corresponding kinase inhibitors (such as epidermal growth factor receptor kinase inhibitors, vascular endothelial growth factor receptor kinase inhibitors, fibroblast growth factor inhibitors, insulin-like growth factor receptor inhibitors and platelet-derived growth factor receptor kinase inhibitors)); methionine, aminopeptidase inhibitors, proteasome inhibitors, cyclooxygenase inhibitors (e.g., cyclooxygenase-1 or cyclooxygenase-2 inhibitors), topoisomerase inhibitors (e.g., topoisomerase I inhibitors or topoisomerase II inhibitors), poly ADP ribose polymerase inhibitors (PARP inhibitors), and epidermal growth factor receptor (EGFR) inhibitors/antagonists.

An alkylating agent which can be used as an anticancer drug in combination with a compound of the present invention may be, for example, a nitrogen mustard (such as cyclophosphamide, mechlorethamine (chlormethine), uramustine, melphalan, chlorambucil, ifosfamide, bendamustine, or trofosfamide), a nitrosourea (such as carmustine, streptozocin, fotemustine, lomustine, nimustine, prednimustine, ranimustine, or semustine), an alkyl sulfonate (such as busulfan, mannosulfan, or treosulfan), an aziridine (such as hexamethylmelamine (altretamine), triethylenemelamine, ThioTEPA (N,N′N′-triethylenethiophosphoramide), carboquone, or triaziquone), a hydrazine (such as procarbazine), a triazene (such as dacarbazine), or an imidazotetrazine (such as temozolomide).

A platinum coordination complex which can be used as an anticancer drug in combination with a compound of the present invention may be, for example, cisplatin, carboplatin, nedaplatin, oxaliplatin, satraplatin, or triplatin tetranitrate.

A cytotoxic drug which can be used as an anticancer drug in combination with a compound of the present invention may be, for example, an antimetabolite, including folic acid analogue antimetabolites (such as aminopterin, methotrexate, pemetrexed, or raltitrexed), purine analogue antimetabolites (such as cladribine, clofarabine, fludarabine, 6-mercaptopurine (including its prodrug form azathioprine), pentostatin, or 6-thioguanine), and pyrimidine analogue antimetabolites (such as cytarabine, decitabine, 5-fluorouracil (including its prodrug forms capecitabine and tegafur), floxuridine, gemcitabine, enocitabine, or sapacitabine).

An antimitotic agent which can be used as an anticancer drug in combination with a compound of the present invention may be, for example, a taxane (such as docetaxel, larotaxel, ortataxel, paclitaxel/taxol, tesetaxel, or nab-paclitaxel (e.g., Abraxane®)), a Vinca alkaloid (such as vinblastine, vincristine, vinflunine, vindesine, or vinorelbine), an epothilone (such as epothilone A, epothilone B, epothilone C, epothilone D, epothilone E, or epothilone F) or an epothilone B analogue (such as ixabepilone/azaepothilone B).

An anti-tumor antibiotic which can be used as an anticancer drug in combination with a compound of the present invention may be, for example, an anthracycline (such as aclarubicin, daunorubicin, doxorubicin, epirubicin, idarubicin, amrubicin, pirarubicin, valrubicin, or zorubicin), an anthracenedione (such as mitoxantrone, or pixantrone) or an anti-tumor antibiotic isolated from Streptomyces (such as actinomycin (including actinomycin D), bleomycin, mitomycin (including mitomycin C), or plicamycin).

A tyrosine kinase inhibitor which can be used as an anticancer drug in combination with a compound of the present invention may be, for example, axitinib, bosutinib, cediranib, dasatinib, erlotinib, gefitinib, imatinib, lapatinib, lestaurtinib, nilotinib, semaxanib, sorafenib, sunitinib, axitinib, nintedanib, ponatinib, or vandetanib.

A topoisomerase inhibitor which can be used as an anticancer drug in combination with a compound of the present invention may be, for example, a topoisomerase I inhibitor (such as irinotecan, topotecan, camptothecin, belotecan, rubitecan, or lamellarin D) or a topoisomerase II inhibitor (such as amsacrine, etoposide, etoposide phosphate, teniposide, or doxorubicin).

A PARP inhibitor which can be used as an anticancer drug in combination with a compound of the present invention may be, for example, BMN-673, olaparib, rucaparib, veliparib, CEP 9722, MK 4827, BGB-290, or 3-aminobenzamide.

An EGFR inhibitor/antagonist which can be used as an anticancer drug in combination with a compound of the present invention may be, for example, gefitinib, erlotinib, lapatinib, afatinib, neratinib, ABT-414, dacomitinib, AV-412, PD 153035, vandetanib, PKI-166, pelitinib, canertinib, icotinib, poziotinib, BMS-690514, CUDC-101, AP26113, XL647, cetuximab, panitumumab, zalutumumab, nimotuzumab, or matuzumab.

Further anticancer drugs may also be used in combination with a compound of the present invention. The anticancer drugs may comprise biological or chemical molecules, like TNF-related apoptosis-inducing ligand (TRAIL), tamoxifen, amsacrine, bexarotene, estramustine, irofulven, trabectedin, cetuximab, panitumumab, tositumomab, alemtuzumab, bevacizumab, edrecolomab, gemtuzumab, alvocidib, seliciclib, aminolevulinic acid, methyl aminolevulinate, efaproxiral, porfimer sodium, talaporfin, temoporfin, verteporfin, alitretinoin, tretinoin, anagrelide, arsenic trioxide, atrasentan, bortezomib, carmofur, celecoxib, demecolcine, elesclomol, elsamitrucin, etoglucid, lonidamine, lucanthone, masoprocol, mitobronitol, mitoguazone, mitotane, oblimersen, omacetaxine, sitimagene, ceradenovec, tegafur, testolactone, tiazofurine, tipifarnib, vorinostat, or iniparib.

Also biological drugs, like antibodies, antibody fragments, antibody constructs (for example, single-chain constructs), and/or modified antibodies (like CDR-grafted antibodies, humanized antibodies, “full humanized” antibodies, etc.) directed against cancer or tumor markers/factors/cytokines involved in proliferative diseases can be employed in cotherapy approaches with the compounds of the invention. Examples of such biological molecules are anti-HER2 antibodies (e.g. trastuzumab, Herceptin®), anti-CD20 antibodies (e.g. Rituximab, Rituxan®, MabThera®, Reditux®), anti-CD19/CD3 constructs (see, e.g., EP1071752) and anti-TNF antibodies (see, e.g., Taylor P C. Antibody therapy for rheumatoid arthritis. Curr Opin Pharmacol. 2003. 3(3):323-328). Further antibodies, antibody fragments, antibody constructs and/or modified antibodies to be used in cotherapy approaches with the compounds of the invention can be found, e.g., in: Taylor P C. Curr Opin Pharmacol. 2003. 3(3):323-328; or Roxana A. Maedica. 2006. 1(1):63-65.

An anticancer drug which can be used in combination with a compound of the present invention may, in particular, be an immunooncology therapeutic (such as an antibody (e.g., a monoclonal antibody or a polyclonal antibody), an antibody fragment, an antibody construct (e.g., a single-chain construct), or a modified antibody (e.g., a CDR-grafted antibody, a humanized antibody, or a “full humanized” antibody) targeting any one of CTLA-4, PD-1/PD-L1, TIM3, LAG3, OX4, CSF1R, IDO, or CD40. Such immunooncology therapeutics include, e.g., an anti-CTLA-4 antibody (particularly an antagonistic or pathway-blocking anti-CTLA-4 antibody; e.g., ipilimumab or tremelimumab), an anti-PD-1 antibody (particularly an antagonistic or pathway-blocking anti-PD-1 antibody; e.g., nivolumab (BMS-936558), pembrolizumab (MK-3475), pidilizumab (CT-011), AMP-224, or APE02058), an anti-PD-L1 antibody (particularly a pathway-blocking anti-PD-L1 antibody; e.g., BMS-936559, MEDI4736, MPDL3280A (RG7446), MDX-1105, or MEDI6469), an anti-TIM3 antibody (particularly a pathway-blocking anti-TIM3 antibody), an anti-LAG3 antibody (particularly an antagonistic or pathway-blocking anti-LAG3 antibody; e.g., BMS-986016, IMP701, or IMP731), an anti-OX4 antibody (particularly an agonistic anti-OX4 antibody; e.g., MEDI0562), an anti-CSF1R antibody (particularly a pathway-blocking anti-CSF1R antibody; e.g., IMC-CS4 or RG7155), an anti-IDO antibody (particularly a pathway-blocking anti-IDO antibody), or an anti-CD40 antibody (particularly an agonistic anti-CD40 antibody; e.g., CP-870,893 or Chi Lob 7/4). Further immunooncology therapeutics are known in the art and are described, e.g., in: Kyi C et al., FEBS Lett, 2014, 588(2):368-76; Intlekofer A M et al., J Leukoc Biol, 2013, 94(1):25-39; Callahan M K et al., J Leukoc Biol, 2013, 94(1):41-53; Ngiow S F et al., Cancer Res, 2011, 71(21):6567-71; and Blattman I N et al., Science, 2004, 305(5681):200-5.

A BRD4 inhibitor (preferably a direct BRD4 inhibitor), such as CeNMIEC2, may also be used as a further therapeutic agent in combination with the compound of formulae (I), (II), (III), (IV) and (V).

The combinations referred to above may conveniently be presented for use in the form of a pharmaceutical formulation. The individual components of such combinations may be administered either sequentially or simultaneously/concomitantly in separate or combined pharmaceutical formulations by any convenient route. When administration is sequential, either the compound of the present invention (particularly the compound of formulae (I), (II), (III), (IV) and (V) or a pharmaceutically acceptable salt, solvate or prodrug thereof) or the further therapeutic agent(s) may be administered first. When administration is simultaneous, the combination may be administered either in the same pharmaceutical composition or in different pharmaceutical compositions. When combined in the same formulation, it will be appreciated that the two or more compounds must be stable and compatible with each other and the other components of the formulation. When formulated separately, they may be provided in any convenient formulation.

The compounds provided herein, particularly the compounds of formulae (I), (II), (III), (IV) and (V), can also be administered in combination with physical therapy, such as radiotherapy. Radiotherapy may commence before, after, or simultaneously with administration of the compounds of the invention. For example, radiotherapy may commence 1-10 minutes, 1-10 hours or 24-72 hours after administration of the compounds. Yet, these time frames are not to be construed as limiting. The subject is exposed to radiation, preferably gamma radiation, whereby the radiation may be provided in a single dose or in multiple doses that are administered over several hours, days and/or weeks. Gamma radiation may be delivered according to standard radiotherapeutic protocols using standard dosages and regimens.

The present invention thus relates to a compound of formulae (I), (II), (III), (IV) and (V) or a pharmaceutically acceptable salt, solvate, or prodrug thereof, or a pharmaceutical composition comprising any of the aforementioned entities in combination with a pharmaceutically acceptable excipient, for use in the treatment or prevention of cancer, wherein the compound or the pharmaceutical composition is to be administered in combination with one or more anticancer drugs and/or in combination with radiotherapy.

Yet, the compounds of formulae (I), (II), (III), (IV) and (V) can also be used in monotherapy, particularly in the monotherapeutic treatment or prevention of cancer (i.e., without administering any other anticancer agents until the treatment with the compound(s) of formulae (I), (II), (III), (IV) and (V) is terminated). Accordingly, the invention also relates to a compound of formulae (I), (II), (III), (IV) and (V) or a pharmaceutically acceptable salt, solvate, or prodrug thereof, or a pharmaceutical composition comprising any of the aforementioned entities in combination with a pharmaceutically acceptable excipient, for use in the monotherapeutic treatment or prevention of cancer.

The subject or patient to be treated in accordance with the present invention may be an animal (e.g., a non-human animal), a vertebrate animal, a mammal, a rodent (e.g., a guinea pig, a hamster, a rat, or a mouse), a canine (e.g., a dog), a feline (e.g., a cat), a porcine (e.g., a pig), an equine (e.g., a horse), a primate or a simian (e.g., a monkey or an ape, such as a marmoset, a baboon, a gorilla, a chimpanzee, an orangutan, or a gibbon), or a human. In accordance with the present invention, it is envisaged that animals are to be treated which are economically, agronomically or scientifically important. Scientifically important organisms include, but are not limited to, mice, rats, and rabbits. Lower organisms such as, e.g., fruit flies like Drosophila melagonaster and nematodes like Caenorhabditis elegans may also be used in scientific approaches. Non-limiting examples of agronomically important animals are sheep, cattle and pigs, while, for example, cats and dogs may be considered as economically important animals. Preferably, the subject/patient is a mammal. More preferably, the subject/patient is a human or a non-human mammal (such as, e.g., a guinea pig, a hamster, a rat, a mouse, a rabbit, a dog, a cat, a horse, a monkey, an ape, a marmoset, a baboon, a gorilla, a chimpanzee, an orangutan, a gibbon, a sheep, cattle, or a pig). Most preferably, the subject/patient is a human.

The term “prevention” of a disorder or disease as used herein (e.g., “prevention” of cancer) is also well known in the art. For example, a patient/subject suspected of being prone to suffer from a disorder or disease may particularly benefit from a prevention of the disorder or disease. The subject/patient may have a susceptibility or predisposition for a disorder or disease, including but not limited to hereditary predisposition. Such a predisposition can be determined by standard methods or assays, using, e.g., genetic markers or phenotypic indicators. It is to be understood that a disorder or disease to be prevented in accordance with the present invention has not been diagnosed or cannot be diagnosed in the patient/subject (for example, the patient/subject does not show any clinical or pathological symptoms). Thus, the term “prevention” comprises the use of a compound of the present invention before any clinical and/or pathological symptoms are diagnosed or determined or can be diagnosed or determined by the attending physician.

It is to be understood that the present invention preferably does not relate to any compounds, be it compounds generally defined by Markush formulae or specific compounds, described in EP 19 20 3702.6, EP 20 17 8833.8, EP 19 20 3697.8 and EP 20 17 8838.7. In particular, the present invention preferably does not relate to any compounds defined by formulae (I) and (II) in EP 19 20 3702.6 and EP 20 17 8833.8 as well as any compounds defined by formula (I) in EP 19 20 3697.8 and EP 20 17 8838.7.

It is to be understood that the present invention specifically relates to each and every combination of features and embodiments described herein, including any combination of general and/or preferred features/embodiments. In particular, the invention specifically relates to each combination of meanings (including general and/or preferred meanings) for the various groups and variables comprised in formulae (I), (II), (III), (IV) and (V).

In this specification, a number of documents including patent applications, scientific literature and manufacturers' manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Cyclin dependent kinases (CDKs) are a family of Ser/Thr kinases that integrate various signal transduction pathways and play a key role in several key cellular processes. CDK12 and its orthologue CDK13 belong to the class of ‘transcriptional’ CDKs. Transcription of protein-coding genes is controlled by RNA Polymerase II. Phosphorylation of residues in its C-terminal domain (CTD) orchestrate the production of mature mRNA transcript. Phosphorylation of Ser2, which promotes elongation of RNA Pol II through the gene body, is a key mechanism of CDK12 transcriptional regulation (Genes & Development 2010, 24:2303-2316). CDK12 and CDK13 associate with their obligate partner Cyclin K to regulate multiple cellular processes, including transcriptional elongation, pre-mRNA splicing, and cell cycle progression. Additionally, CDK12 knockdown has been associated with downregulation of genes involved in homologous recombination and the DNA damage response (DDR) (Genes & Development 2011, 25:2158-2172). Hence, maintenance of genomic stability appears to be a key role of this protein.

CDK12 is often dysregulated in human cancers and is an attractive therapeutic target. Mutation of CDK12 in serious ovarian carcinoma is associated with decreased expression of DDR genes such as BRCA1, FANCI, ATM, ATR or FANCD2 and increased sensitivity to PARP inhibitors. (Cancer Res, 2016, 76(7) 1182; Nucleic Acids Research, 2015, Vol. 43, 2575-2589).

The frequency and distribution of CDK12 protein expression was assessed by immunohistochemistry (IHC) in independent cohorts of breast cancer and this was correlated with outcome and genomic status. It was found that 21% of primary unselected breast cancers were CDK12 high, and 10.5% were absent. CDK12 overexpression in breast cancer cells has been demonstrated to regulate splicing of pre-mRNA involved in DDR and tumorigenesis. (Nucleic Acids Res., 2017, Jun. 20; 45(11):6698-6716). Disruption of Cyclin-Dependent Kinase 12 (CDK12) is known to lead to defects in DNA repair and sensitivity to platinum salts and PARP1/2 inhibitors. Interestingly, absence of CDK12 protein was associated with reduced expression of a number of DDR proteins including ATR, Ku70/Ku80, PARP1, DNA-PK, and γH2AX, suggesting a novel mechanism of CDK12-associated DDR dysregulation in breast cancer. This may have important therapeutic implications, particularly for triple-negative breast cancers. (Molecular Cancer Therapeutics (2018), 17(1), 306-315).

Human epidermal growth factor receptor 2 (HER2) is a member of the epidermal growth factor receptor family having tyrosine kinase activity. Amplification or overexpression of HER2 occurs in approximately 15-30% of breast cancers and 10-30% of gastric/gastroesophageal cancers and serves as a prognostic and predictive biomarker. HER2 overexpression has also been seen in other cancers like ovary, endometrium, bladder, lung, colon, and head and neck. The introduction of HER2 directed therapies has dramatically influenced the outcome of patients with HER2 positive breast and gastric/gastroesophageal cancers (Mol Biol Int. 2014; 2014: 852748). In breast cancer, HER2 is a part of the frequently amplified and overexpressed 17q12-q21 locus. 17q12-q21 amplicon commonly contains several neighboring genes including MED1, GRB7, MSL1, CASC3 and TOP2A. The HER2 amplicon also contains the CDK12 gene in 71% of cases (Cell Division, Volume 12, Article number: 7 (2017)). High CDK12 expression caused by concurrent amplification of CDK12 and HER2 in breast cancer patients is associated with disease recurrence and poor survival (EMBO Rep (2019)20:e48058).

The design of selective ATP-competitive kinase inhibitors is challenging, due to the similarity of the ATP binding sites, as well as difficulties in overcoming the overwhelmingly high intracellular concentrations of ATP. To date, all CDK12 inhibitors in clinical trials are pan-CDK inhibitors (Dinaciclib). As an alternative to classical competitive inhibition, degradation of the target of interest is therefore an attractive alternative, especially if such degraders can overcome common problems of ATP competitive kinase inhibitors such as poor permeability, low oral availability, poor CNS penetration, and high levels of P-gp and BCRP1 mediated efflux.

The present invention relates to compounds that cause degradation of Cyclin K via a “molecular glue” mechanism and consequently selective inactivation of CDK12 and CDK13. This is achieved via stabilization of an interaction between a CDK12/Cyclin K complex and a Cullin-RING E3 ligase (CRL). CRLs are multi-subunit complexes composed of a Cullin scaffold (e.g. CUL1, CUL2, CUL3, CUL4A, CUL4B, CUL5, CUL7, CUL9) and a substrate receptor (SR) conferring target specificity to the complex (e.g. CRBN, VHL, DCAF15) recruited via an adaptor subunit (e.g. DDB1, SKP1, ELOB/C). A target protein presented by the SR is tagged for proteasomal degradation via transfer of ubiquitin by E2 enzymes recruited to the CRL. For the present invention, the CDK12/Cyclin K interacts with a CRL complex comprises CUL4A or CUL4B and DDB1. CDK12 directly binds to DDB1 and acts as a surrogate SR to expose Cyclin K for ubiquitination.

Cyclin K degradation is a property that has been described for some, but not all inhibitors of CDK12. Interaction between CDK12 and DDB1 is driven, in part, due to interactions of the inhibitor with DDB1. Therefore, only CDK12 inhibitors that simultaneously occupy the kinase active site and fill the hydrophobic pocket of DDB1 can promote Cyclin K degradation. For example, the pan-CDK inhibitor CR8 was found to cause Cyclin K degradation by this mechanism, whereas the CDK12 selective covalent inhibitor THZ-531 did not cause cyclin K degradation. However, prediction of Cyclin K degradation properties of a CDK12 inhibitor or design of a Cyclin K degrader are not obvious. Consequently, the Cyclin K degraders reported in the literature have been discovered serendipitously.

Inhibitors of CDK12 catalytic activity require sustained occupancy of the kinase by the drug to maintain the inhibitory effect. In contrast, since transient interaction of CDK12/Cyclin K and the CRL is sufficient to drive Cyclin K degradation, the compounds in the present invention act through a catalytic, event-based pharmacology. Therefore, pronounced inactivation of CDK12 can be achieved at significantly lower drug concentrations. This is supported by the potency of these molecules in cell viability assays in a leukemic cell line (KBM7). We further profiled the compounds in a previously described isogenic cell line with impaired CRL activity (UBE2Mmut, Mayor-Ruiz et al, 2020) which display reduced Cyclin K degradation activity, whereas inhibition of CDK12 catalytic activity is unaffected. Sensitivity in these isogenic cancer cell line pair differs by 10-150-fold, underscoring the important contribution of Cyclin K degradation to the therapeutic effect. Since the dose required for Cyclin K degradation is orders of magnitude lower than for ATP-competitive inhibition of CDK12, these molecules are also anticipated to have a strongly improved selectivity profile over classical inhibitors. In line with this, compounds with similar inhibitory effects on CDK12 catalytic activity can display marked differences in cellular efficacy thanks to the added effect of Cyclin K degradation (compare e.g. examples 3-36 and 3-57).

It is believed that CDK12 and CDK13 share a largely overlapping target space (Liang et al, 2015) and therefore CDK13 is able to compensate loss of CDK12 enzymatic activity. Cyclin K is the obligate partner for both CDK12 and CDK13 and is needed for their activity. Cyclin K degraders will therefore cause impaired activity of both kinases, potentially circumventing such compensatory signaling.

Restoration of CDK12 activity upon treatment with Cyclin K degraders requires the re-synthesis of Cyclin K. Cyclin K is a relatively long-lived protein with a reported half-life >12 hours. Hence, the compounds object of the present invention are expected to have a therapeutic effect in cells and tumors that extends well beyond exposure to the molecule. This favorable disconnect between pharmacokinetics and pharmacodynamics can be exploited to further optimize the selectivity profile of these molecules and reduce the dosing schedule.

Emergence of drug resistance is a common pitfall of targeted cancer therapies. Resistance to kinase inhibitors in particular is often mediated by accumulation of mutations in the active site of the enzyme, so-called “gatekeeper mutations”, that reduce the binding affinity of the drug and consequently its occupancy of the target kinase. Due to their more efficient binding mode and mechanism of action, our Cyclin K degraders are envisaged to be able to circumvent these common resistance mutations. Furthermore, it has been hypothesized that because of the catalytic mode of action of degraders, larger drops in affinity are required for loss of therapeutic effect of a degrader compared to an inhibitor.

A described resistance mechanism of degraders is downregulation or mutation of the SR required for degradation, as loss of its function does not typically confer a loss of fitness to the cancer cells. The Cyclin K degraders described herein do not utilize a canonical SR but rather co-opt CDK12 as a surrogate SR which is directly recruited to DDB1 (as shown by nanoBRET ternary complex formation data), which is pan-essential across cell types. Therefore, interference with the functioning of this CRL complex would likely result in a considerable loss of fitness and be disfavored as a potential resistance mechanism.

Compounds of the invention have a number of exceptional features anticipated to make them especially useful in the treatment of cancer. Their low molecular weight, optimal lipophilicity, and low number of hydrogen bond donors and acceptors is anticipated to lead to low levels of transporter-mediated efflux and better blood-brain barrier penetration than observed for other described CCNK degraders (Wager et al., ACS Chemical Neuroscience, 2010 (1), p. 435). Such characteristics will make compounds of the invention especially suitable for use in brain cancers and cancers that have spread to the brain. Brain metastases are frequently observed in lung, breast and skin cancers, and it is anticipated that compounds of the invention will be especially useful in such situations.

The compounds of the invention also show very high aqueous solubility, which in combination with high levels of permeability and metabolic stability is expected to result in very high oral availability. High solubility also allows for intravenous formulations and parenteral delivery of the compounds of the invention for patients unable to take medicines by mouth.

The most preferred compounds of the invention are distinguished by the presence of a hydrogen bond acceptor atom in the bicyclic ring system R1. High levels of Cdk selectivity are expected for such compounds due to a specific interaction with the tyrosine-815 residue of Cdk12. This residue is not conserved across the Cdk family—the equivalent residue in Cdk2, Cdk7 & Cdk9 is phenylalanine, in Cdk4 is histidine. It is anticipated that higher levels of selectivity for binding to Cdk12 will result from such specific (or water-mediated) interactions between the ligand and the protein, resulting in lower off-target mediated toxicity.

The compounds of the invention are also especially potent degraders of CCNK, likely permissive of low clinical dosing with resulting improvements in tolerability and lower levels of toxicity. Furthermore, it is anticipated that low levels of protein binding will lead to high unbound (Cₘₐₓ) drug concentrations in vivo, beneficial for the event-driven pharmacology expected.

The activity and mechanism of action has been elucidated using, i.a., the following assays:

Cell Titer Glo (CTG) in KBM7 WT and UBE2Mmut:

This is a cell viability assay to measure the desired therapeutic effect, i.e. killing of cancer cells. The UBE2M mutant cell line has impaired activity of the Cullin-RING ligase system due to a hypomorphic mutation in UBE2M, which is required for activation of these E3 ligases. By comparison with WT cells, it is possible to estimate the contribution of Cyclin K degradation (which is reduced in UBE2M mut cells) compared to kinase inhibition of CDK12 (which is unaffected in UBE2M mut cells). Since the UBE2M mutation only reduces CRL function without fully abrogating it, this assay may slightly underestimate the effect of degradation because, at high compound concentrations, degradation may still be observed.

CCNK-Nanoluciferase Degradation Assay:

The assay helps to elucidate the mechanistic explanation of the cell killing effect of the compounds of the present invention. Cyclin K is fused to a nanoluciferase, light emission is measured as a proxy of Cyclin K abundance. Potency of Cyclin K degradation and cytotoxicity of KBM7 correlate extremely well, further supporting Cyclin K degradation being the main driver of the cell killing, as opposed to inhibition of CDK12 catalytic activity.

nanoBRET Assay for DDB1-CDK12/Cyclin K Ternary Complex Formation:

This assay measures the interaction/recruitment of CDK12/Cyclin K to DDB1, mechanistically validating the molecular glue pharmacology. Since the distance at which the interaction is measurable with this assay is quite small (<10 nm), this assay further indicates that the interaction between CDK12 and DDB1 is direct, without a canonical Substrate Receptor being required.

Recombinant CDK12 Kinase Inhibition Assay:

This assay on recombinant proteins allows assessment of the extent of inhibition of CDK12 kinase activity of compounds. The added benefit of Cyclin K degradation as opposed to CDK12 inhibition alone can thereby be estimated. The observed disconnect between CDK12 inhibition and efficacy in the KBM7 cell viability assay supports an important contribution of Cyclin K degradation to the therapeutic effect.

The Figures show

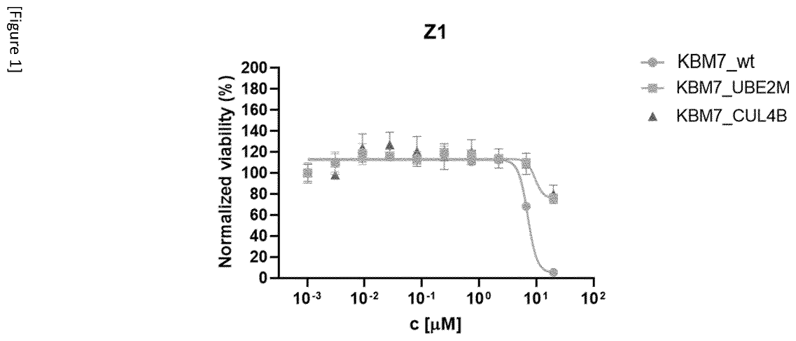

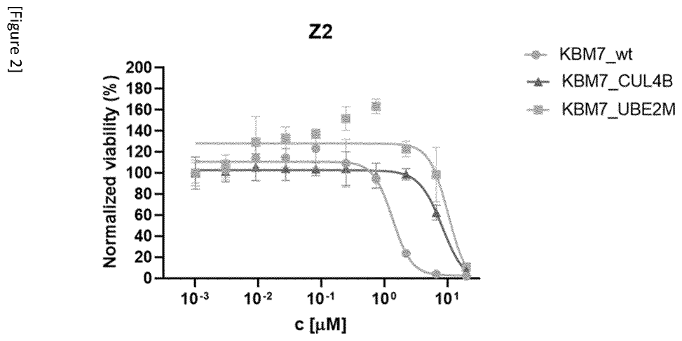

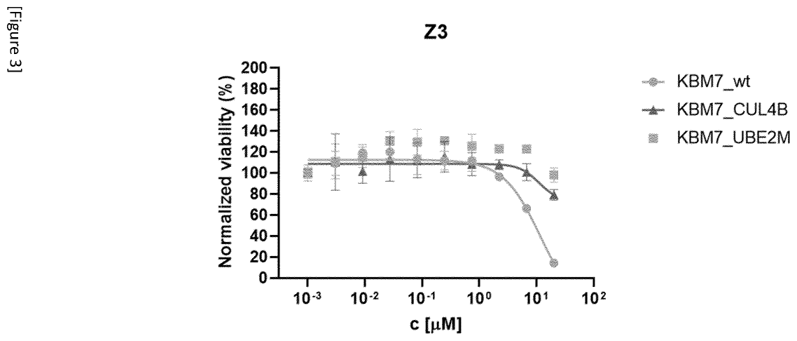

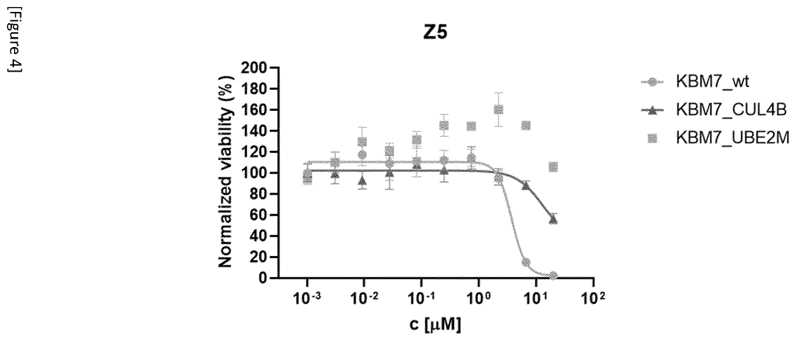

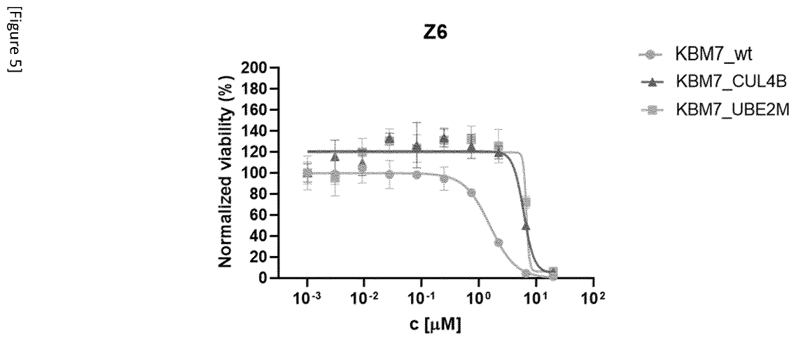

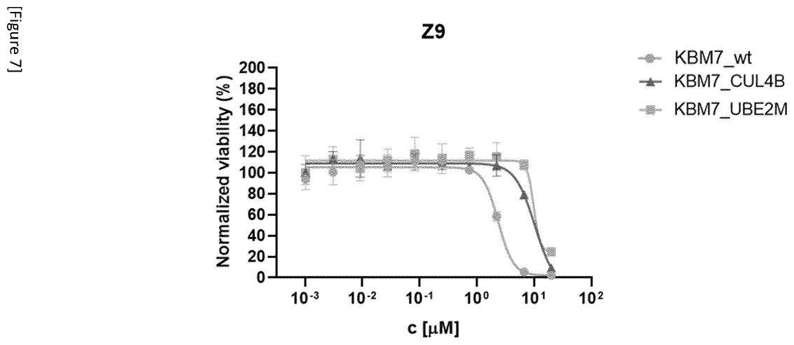

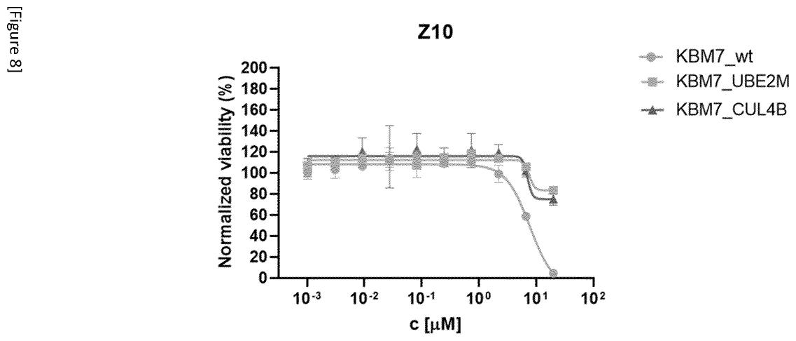

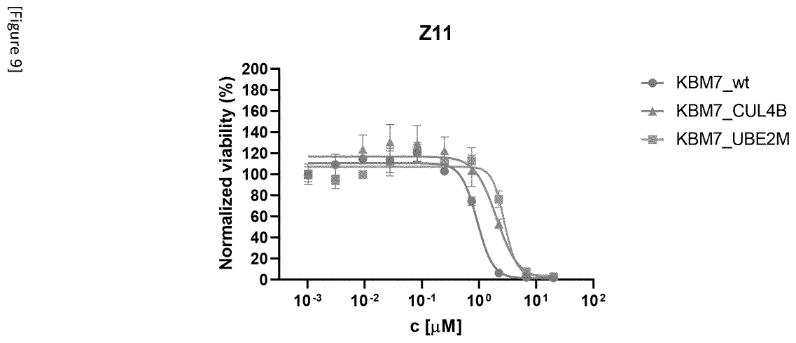

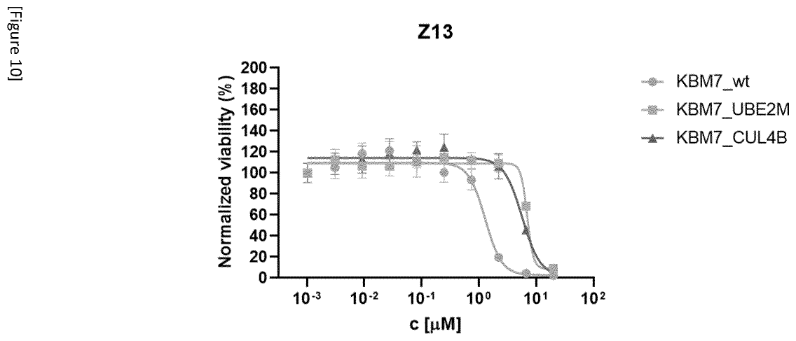

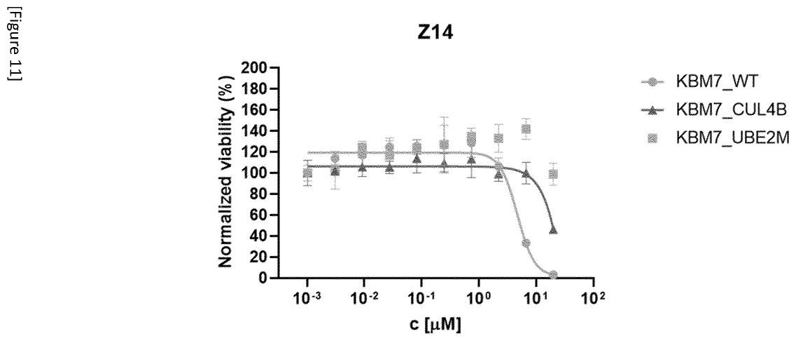

FIG. 1 to 11. Dose-ranging viability data of selected compounds Z1 to Z3, Z5 to Z7, Z9 to Z11, Z13 and Z14 in the indicated genetic backgrounds. Dose-ranging viability data show DMSO-normalized cellular viability after 3-day hit treatment in KBM7^{WT}, CUL4B^{mut} and UBE2M^{mut} cells.

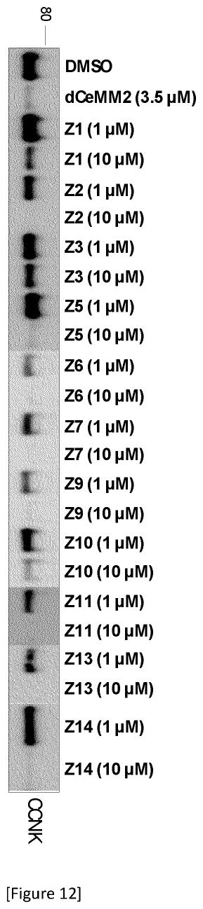

FIG. 12. Degradation of CCNK levels upon Z1 to Z3, Z5 to Z7, Z9 to Z11, Z13 and Z14 treatment in KBM7^{WT} cells.

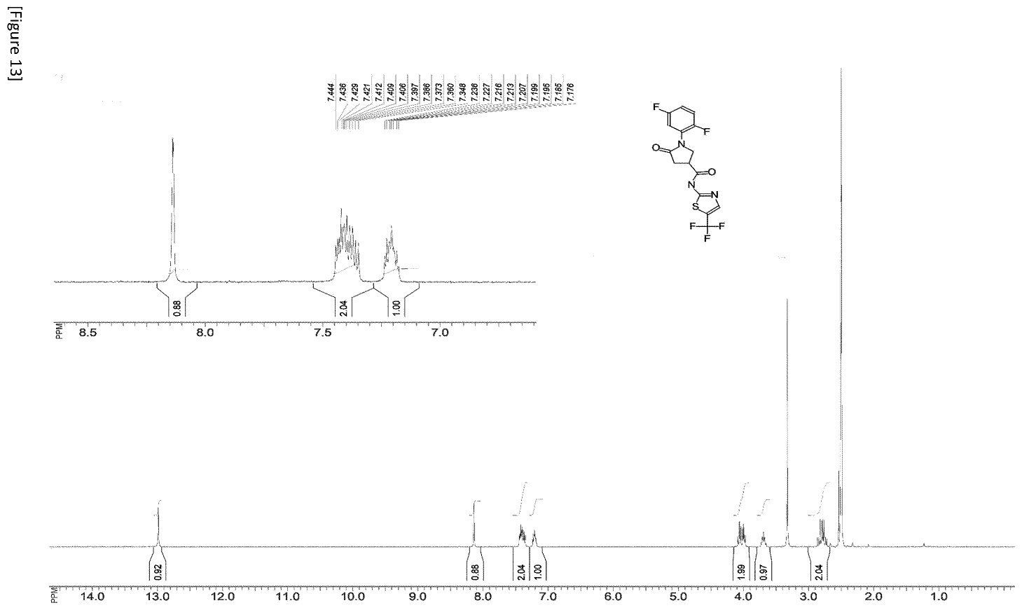

FIG. 13. ¹H NMR of Example 3-22

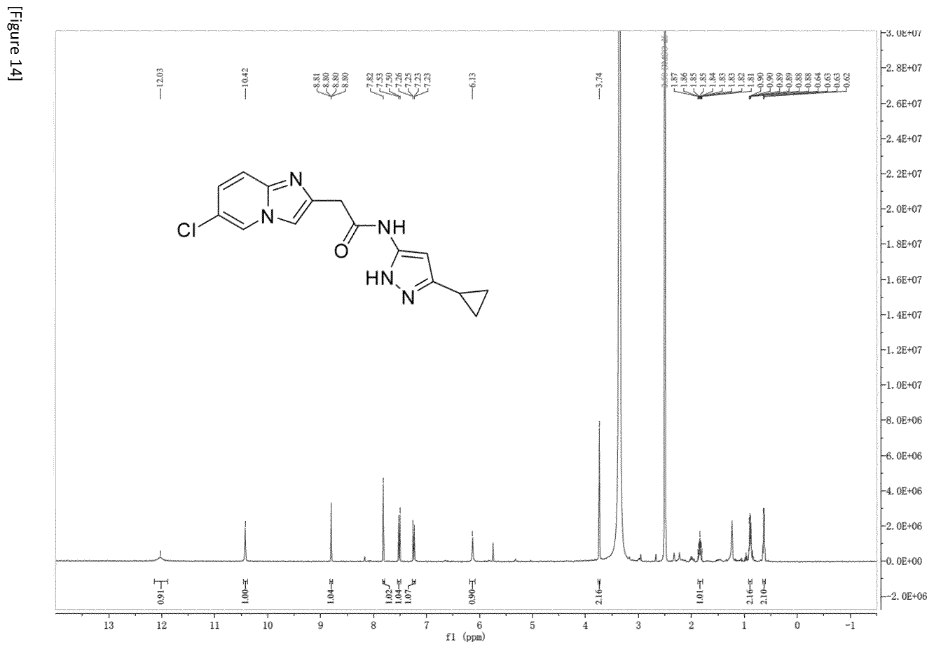

FIG. 14. ¹H NMR of Example 3-66

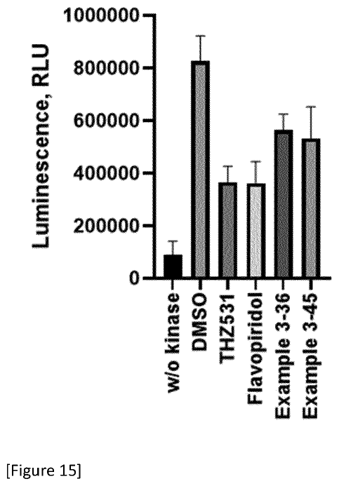

FIG. 15. Cdk12 [high ATP] in vitro kinase activity assay of Examples 3-36 and 3-45

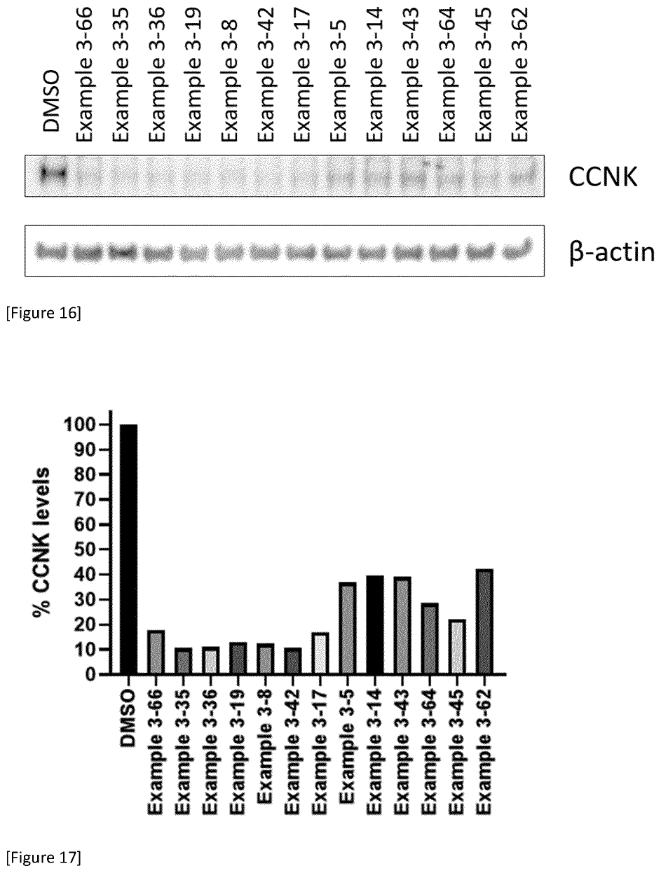

FIG. 16. Western blot analysis of CCNK degradation.

FIG. 17. Percentage CCNK remaining 4 h 10 micromolar treatment as evaluated by the densitometry values normalised to actin levels using ImageJ

### EXAMPLES

All synthesis was carried out at Enamine Ltd (Kyiv, Ukraine; https://www.enaminestore.com/catalog) or at Chembridge (San Diego, CA; https://www.hit2lead.com/screening-compounds/9083792) from commercially available building blocks.

Particularly, compounds “Z1 to Z3, Z5 zo Z7, Z9 to Z11 and Z14” were synthesized at Enamine Ltd: Product ID of compound “Z1”: Z28172116; Product ID compound “Z2”: Z63439346; Product ID of compound “Z3”: Z300783508; Product ID of compound “Z5”: Z397749190; Product ID of compound “Z6”: Z104866096; Product ID of compound “Z7”: Z200170434; Product ID of compound “Z9”: Z336658234; Product ID of compound “Z10”: Z1419842998; Product ID of compound “Z11”: Z27665843; Product ID of compound “Z14”: Z381246898. Compound “Z13” was synthesized at Chembridge: Product ID of compound “Z13”: 908379.

### General Methods

### Cell Lines

KBM7 cells with the specified genetic backgrounds were grown in IMDM supplemented with 10% FBS and 1% penicillin/streptomycin (pen/strep). AsPC1, HCT116, NCI-H446 and 293T cells were grown in DMEM 10% FBS and 1% pen/strep. MV4; 11, Jurkat and Be(2)C cells were grown in RPMI 10% FBS 1% pen/strep. KBM7, AsPC1 and HCT116 cells expressing Cas9 were generated using the plasmid Lenti_Cas9_Blasti (Addgene #52962). The lentiviral plasmid lentiGuide-Puro (Addgene #52963) was used to express sgRNAs against the genes UBE2M (in KBM7-Cas9, AsPC1-Cas9 and HCT116-Cas9 cells). The lentiviral plasmid lentiGuide-Puro-IRES-mCherry (modified from Addgene #52963) was used to express sgRNAs against CUL4B (in KBM7-Cas9 cells). The lentiviral plasmid pLenti-PGK-Hygro-DEST-UBE2M was generated by Gateway cloning (empty destination vector Addgene #19066), and used to generate UBE2M^{rese} KBM7 cells.

| sgRNA name | sequence |
|---|---|
| sgUBE2M (SEQ ID NO. 3) | TCACCCCAACATTGACCTCG |
| sgCUL4B (SEQ ID NO. 6) | AGCATGTGGTACTTACTGGG |

### Example 1: Identification of Cullin RING Ubiquitin Ligase Modulators

### Study Design

All known small molecule degraders (heterobifunctional PROTACs as well as molecular glues) require the activity of the pan CRL regulator UBE2M. Depending on the hijacked CRL ligase, compounds also require the activity of selected cullin backbones, for instance CUL4B. In other words: cancer cells where UBE2M have been mutated via CRISPR/Cas9 technology are usually insensitive to the anti-cancer properties of these degraders, while mutation of CUL4B will render a subset of the degraders inactive.

### Methods of Cell Viability Assay as Shown in FIG. 1 to 11

KBM7^{WT}, UBE2M and CUL4B mutant KBM7 clones (UBE2M^{mut} and CUL4B^{mut}) were seeded at a cell density of 50,000 cells/mL in 96-well with DMSO or degraders Z1 to Z3, Z5 to Z7, Z9 to Z11, Z13 and Z14 in triplicates. Cells were treated for 3 days, after which a cell viability assay was performed (CellTiter Glo, Promega), according to manufacturer's protocol. Survival curves and IC₅₀ values (LC₅₀ values) were calculated by best-fit analysis of the log 10 drug concentration to fold change of drug-treated cells over DMSO-treated cells. All survival assays included technical triplicates per sample, per experiment.

Compounds, Z1 to Z3, Z5 to Z7, Z9 to Z11, Z13 and Z14, were tested for their antiproliferative effects in human leukemia cells (KBM7). These cells were either transduced with a control sgRNA (KBM7_WT), or with an sgRNA targeting UBE2M or CUL4B, leading to a hypomorph (deletion of six amino acids) with a functional impairment (UBE2M_MUT). Thus, a putative novel degrader would potently inhibit the proliferation of KBM7_WT cells, while sparing UBE2Mmut isogenic counterparts.

Indeed, 11 compounds Z1 to Z3, Z5 to Z7, Z9 to Z11, Z13 and Z14 were identified to fulfill these criteria and to stand out in terms of fold-change significance; see, e.g., FIG. 1 and Table 1. The dose ranging differential effect of these hits was assessed/validated, comparing their activity in KBM7_WT (KBM7^{WT}) vs UBE2_MUT and CUL4B_MUT cells (UBE2M^{mut} and CUL4B^{mut}). All assayed compounds showed a pronounced shift in their anti-proliferative effects upon mutation of UBE2M or CUL4B, as observed by a shift in their half maximal inhibitory concentration (LC₅₀); see Table 1.

TABLE 1
LC₅₀ values (μM) of data depicted in FIG. 1 to 11

| Compound | Compounds | LC₅₀ | LC₅₀ | LC₅₀ |
|---|---|---|---|---|
| name | Structure | KBM7_WT | UBE2M_MUT | CUL4B_MUT |
| Z1 | 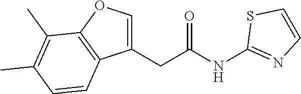 [SMILES: Cc1ccc2c(CC(=O)Nc3nccs3)coc2c1C] | 7.121 | >10 | >10 |
| Z2 | 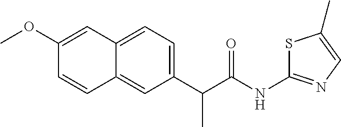 [SMILES: COc1ccc2cc(C(C)C(=O)Nc3ncc(C)s3)ccc2c1] | 1.350 | >10 | >10 |
| Z3 | 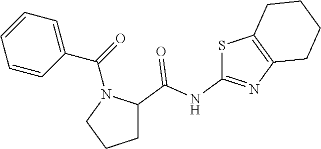 [SMILES: O=C(Nc1nc2c(s1)CCCC2)C1CCCN1C(=O)c1ccccc1] | 11.750 | >10 | >10 |
| Z5 | 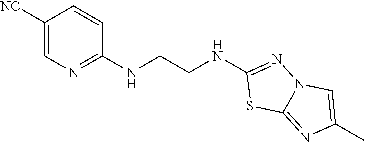 [SMILES: Cc1cn2nc(NCCNc3ccc(C#N)cn3)sc2n1] | 3.747 | >10 | >10 |
| Z6 | 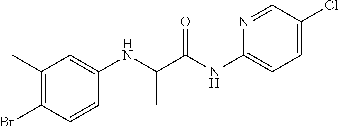 [SMILES: Cc1cc(NC(C)C(=O)Nc2ccc(Cl)cn2)ccc1Br] | 1.569 | 6.797 | 6.061 |
| Z7 | 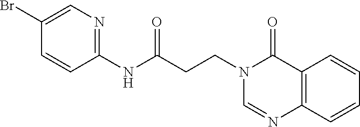 [SMILES: O=C(CCn1cnc2ccccc2c1=O)Nc1ccc(Br)cn1] | 1.812 | >10 | >10 |
| Z9 | 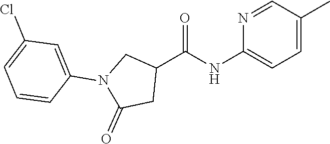 [SMILES: Cc1ccc(NC(=O)C2CC(=O)N(c3cccc(Cl)c3)C2)nc1] | 2.352 | >10 | >10 |
| Z10 | 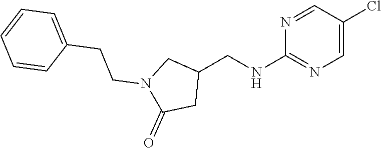 [SMILES: O=C1CC(CNc2ncc(Cl)cn2)CN1CCc1ccccc1] | 7.821 | >10 | >10 |
| Z11 | 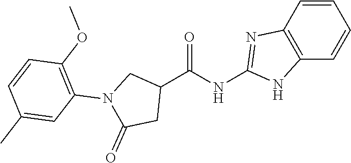 [SMILES: COc1ccc(C)cc1N1CC(C(=O)Nc2nc3ccccc3[nH]2)CC1=O] | 0.920 | 2.748 | 2.005 |
| Z13 | 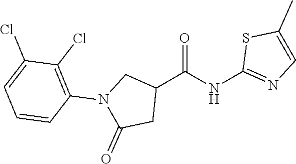 [SMILES: Cc1cnc(NC(=O)C2CC(=O)N(c3cccc(Cl)c3Cl)C2)s1] | 1.286 | 6.993 | 5.682 |
| Z14 | 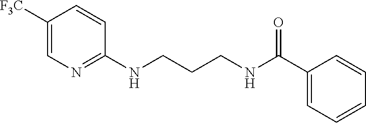 [SMILES: O=C(NCCCNc1ccc(C(F)(F)F)cn1)c1ccccc1] | 4.693 | >10 | >10 |

### Example 2: Identification of Novel, Structurally Distinct Compounds Degrading Cyclin K

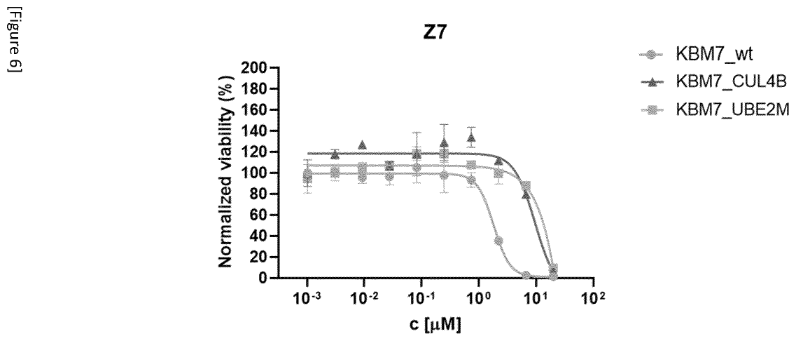

The hypomorphic phenotype of the mutated UBE2M and CUL4B allele in KBM7 cells (KBM7 UBE2M^{mut} and CUL4B^{mut} cells) as used in Example 1 was assessed. Cellular treatment of the mutated cells with the compounds Z1 to Z3, Z5 to Z11, Z13 and Z14 as shown in FIG. 6 in comparison with wild-type KBM7 cells revealed that these compounds led to a destabilization of cyclin K (CCNK) (FIG. 12).

The degradation of CCNK in KBM7_WT cells via the E3 ligase is the reason for the loss in cancer cell viability after compound treatment. Based on the E3 ligase dependent mode of action of the compounds as disclosed above and shown in the appended Examples below, it is anticipated that the degradation of CCNK by the compounds Z1 to Z3, Z5 to Z11, Z13 and Z14 can also be induced by binding of the compounds to CDK12. CDK12 in turn leads to degradation of CDK12-associated CCNK via the E3 ligase.

### Western Blot Analysis

PBS-washed cell pellets were lysed in 50 mM Tris pH 7.9, 8M Urea and 1% CHAPS and incubated with shaking at 4° C. for at least 30 min. 20 g of supernatants were run and transferred for detection. Antibodies used: CUL1 (Santa Cruz Biotechnology, sc-1276), CUL2 (Sigma-Aldrich, SAB2501565-100), CUL3 (Cell Signaling Technology, 2759), CUL4A (Cell Signaling Technology, 2699S), CUL4B (Proteintech, 12916-1-AP), CUL5 (Santa Cruz Biotechnology, sc-373822), UBE2M (Santa Cruz Biotechnology, sc-390064), DDB1 (Cell Signaling Technology, 5428S), CCNK (Bethyl, A301-939A), CDK12 (Cell Signaling Technology, 11973S), CDK13 (Bethyl, A301-458A), RBM39 (1:500, Santa Cruz Biotechnology sc-376531), V5 (Cell Signaling Technology, 13202), Ubiquityl-Histone H2A (K119) (Cell Signaling, 8240-20). ACTIN (Sigma-Aldrich, A5441), VINCULIN (Santa Cruz Biotechnology, sc-25336). Secondary antibodies anti-mouse/rabbit/goat (Jackson ImmunoResearch 115-035-003, 111-035-003 and 705-035-003).

### Example 3: Synthesis and Testing of Further Compounds

### Synthesis of Compounds—Method A

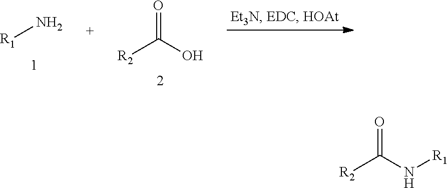

Reagent 1 (1 eq.), reagent 2 (1.1 eq.), and 1-ethyl-3-(3-dimethylamino propyl) carbodiimide (EDC) (1.1 eq.), and 1-hydroxy-7-azabenzotriazole (HOAt) (1.05 eq.) were mixed in dry N,N-dimethylformamide (DMF, approximately 0.5 ml per 100 mg of product). The reaction mixture was sealed and left at ambient temperature for 18 hours. Then the solvent was evaporated under reduced pressure and the residue was dissolved in DMSO (appr. 1 ml up to 300 mg of product). The DMSO solution was filtered, analyzed by LCMS, and transferred for HPLC purification.
In case of using Reagent 1 and/or Reagent 2 as salt, an additional amount of triethylamine (Et₃N) (1.1 eq.) was added to the reaction mixture to convert the reagent into a free form.
In case of using Reagent 2 as salt, an additional amount of triethylamine hydrochloride (TETAH) (1.1 eq.) was added to the reaction mixture to convert the reagent into the base form.

### Synthesis of Compounds—Method B

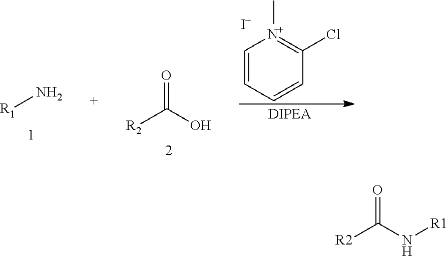

A vial was charged with reagent 2 (1.2 eq.) and dry acetonitrile (MeCN) (1 mL). N,N-diisopropylethylamine (DIPEA*, 3.2 eq.) was added dropwise to the solution. A mixture of reagent 1 (1 eq.) and 2-chloro-N-methylpyridinium iodide (1.44 eq.) was added with stirring. The reaction vial was placed into a water bath and left at 100° C. for 6 h. The reaction mixture was cooled to room temperature and water was added until the vial was full. Then the vial was sonicated in an ultrasonic bath. The solvent was evaporated. The residue was dissolved in DMSO and filtered. The solution was subjected to HPLC purification.
In case of using amine salt, an additional amount of N,N-diisopropylethylamine (DIPEA) (1.1 eq.) was added to the reaction mixture to convert the amine into the base form.

### Synthesis of Compounds—Method C

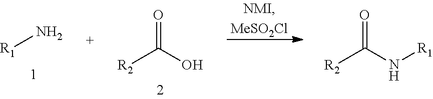

Reagent 2 (1.2 eq.) and N-methylimidazole (NMI, 2 eq.) were mixed in dry acetonitrile (MeCN, approximately 0.7 ml per 100 mg of product). Methanesulfonyl chloride (MeSO₂Cl) (1. eq.) was added dropwise with stirring for 5 min. The mixture was sealed and heated with stirring for 1 hour at 50° C. and after cooling reagent 1 (1 eq.) was added in one portion. Then the reaction mixture was sealed and heated for 16 hours at 60° C. The mixture was cooled to ambient temperature, the solvent was evaporated under reduced pressure, and the residue was dissolved in the DMSO (approximately 1 ml up to 300 mg of product). The DMSO solution was filtered, analyzed by LCMS, and transferred for HPLC purification.

### Synthesis of Compounds—Method D

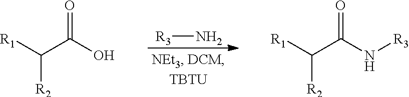

To a cooled (0° C.) solution of the acetic acid (1.62 mmol), amine (1.62 mmol) and triethylamine (0.68 mL, 4.87 mmol), in dichloromethane (15 mL) was added TBTU (2-(1H-benzotriazole-1-yl)-1,1,3,3-tetramethyluronium tetrafluoroborate) (625 mg, 1.95 mmol). The resulting solution was stirred at room temperature for 22 h. The reaction mixture was evaporated to dryness and the residue was purified by flash chromatography or HPLC to yield the final compound.

### Synthesis of Compounds—Method E

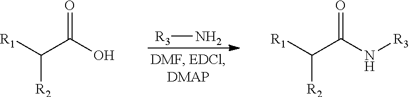

Chemists skilled in the art will appreciate that similar methods to those shown in Methods A-E are suitable for the synthesis of compounds of the invention.

### Compound Identification: NMR

For a number of compounds, NMR spectra were recorded either using a Bruker DPX400 spectrometer equipped with a 5 mm reverse triple-resonance probe head operating at 400 MHz for the proton and 100 MHz for carbon or using a Bruker DRX500 spectrometer equipped with a 5 mm reverse triple-resonance probe head operating at 500 MHz for the proton and 125 MHz for carbon. Deuterated solvents were chloroform-d (deuterated chloroform, CDCl₃) or d6-DMSO (deuterated DMSO, d6-dimethylsulfoxide). Chemical shifts are reported in parts per million (ppm) relative to tetramethylsilane (TMS) which was used as internal standard.

### Compound Identification: HPLC/MS

For a number of compounds, LC-MS spectra were recorded using the following instruments and analytical methods.

### Instrument Specifications:

Agilent 1100 Series LC/MSD system with DAD\ELSD Alltech 2000ES and Agilent LC\MSD VL (G1956B), SL (G1956B) mass-spectrometer

Agilent 1200 Series LC/MSD system with DAD\ELSD Alltech 3300 and Agilent LC\MSD G6130A, G6120B mass-spectrometer.

Agilent Technologies 1260 Infinity LC/MSD system with DAD\ELSD Alltech 3300 and Agilent

LC\MSD G6120B mass-spectrometer.

Agilent Technologies 1260 Infinity II LC/MSD system with DAD\ELSD G7102A 1290 Infinity II and Agilent LC\MSD G6120B mass-spectrometer.

Agilent 1260 Series LC/MSD system with DAD\ELSD and Agilent LC\MSD (G6120B) mass-spectrometer.

UHPLC Agilent 1290 Series LC/MSD system with DAD\ELSD and Agilent LC\MSD (G6125B) mass-spectrometer.

### Method A

Column: Agilent Poroshell 120 SB-C18 4.6×30 mm 2.7 μm with UHPLC Guard Infinity Lab Poroshell 120 SB-C18 4.6×5 mm 2.7 μm

Temperature: 60° C.

Mobile phase A: acetonitrile:water (99:1%), 0.1% formic acid

Mobile phase B: water (0.1% formic acid)

Flow rate: 3 ml/min

Elution Gradient: 0.01 min—99% B, 1.5 min—0% B, 2.2 min: 0% B, 2.21 min: 99% B

Injection volume: 0.5 μl

Ionization mode: Electrospray ionization (ESI)

Scan range: m/z 83-1000

DAD: 215 nm, 254 nm, 280 nm

### Method B

Column: Agilent Poroshell HPH-C18, 4.6*100 mm, 4 um

Temperature: 35° C.

Mobile phase A: water, 0.1% TFA

Mobile phase B: acetonitrile

Flow rate: 1 ml/min

Elution Gradient: 0.01 min—90% A, 1 min—90% A, 17 mins 100% B, 18 mins—100% B

Injection volume: 5 μl

Ionization mode: Electrospray ionization (ESI)

Scan range: m/z 83-1000

### Example 3-1: 2-(6,7-dimethylbenzofuran-3-yl)-N-(5-methylthiazol-2-yl)acetamide

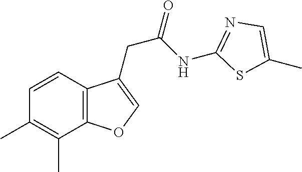

Prepared according to Method A.

LCMS Retention time (Method A): 1.427 minutes; m/z 301.2 (M+H)⁺

### Example 3-2: 2-(6,7-dimethylbenzofuran-3-yl)-N-(5-isopropylthiazol-2-yl)acetamide

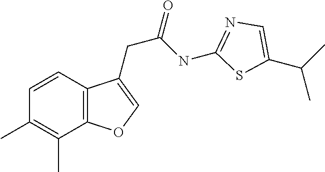 [SMILES: Cc1ccc2c(CC(=O)Nc3ncc(C(C)C)s3)coc2c1C]

Prepared according to Method A.

LCMS Retention time (Method A): 1.356 minutes; m/z 329.2 (M+H)⁺

### Example 3-3: N-(5-chlorothiazol-2-yl)-2-(6,7-dimethylbenzofuran-3-yl)acetamide

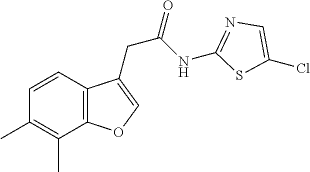 [SMILES: Cc1ccc2c(CC(=O)Nc3ncc(Cl)s3)coc2c1C]

Prepared according to Method A.

LCMS Retention time (Method A): 1.468 minutes; m/z 321.0/323.1 (M+H)⁺

### Example 3-4: 2-(6-fluoroimidazo[1,2-a]pyridin-2-yl)-N-(5-isopropylthiazol-2-yl)acetamide

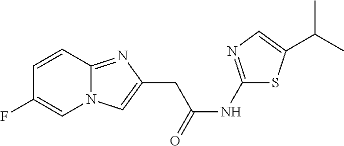 [SMILES: CC(C)c1cnc(NC(=O)Cc2cn3cc(F)ccc3n2)s1]

Prepared according to Method A.

LCMS Retention time (Method A): 0.727 minutes; m/z 319.0 (M+H)⁺

### Example 3-5: 2-(6-fluoroimidazo[1,2-a]pyridin-2-yl)-N-(5-(trifluoromethyl)thiazol-2-yl)acetamide

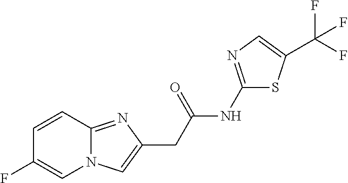 [SMILES: O=C(Cc1cn2cc(F)ccc2n1)Nc1ncc(C(F)(F)F)s1]

Prepared according to Method B.

LCMS Retention time (Method A): 0.989 minutes; m/z 345.0 (M+H)⁺

### Example 3-6: 2-(8-methylimidazo[1,2-a]pyridin-3-yl)-N-(5-(trifluoromethyl)thiazol-2-yl)acetamide

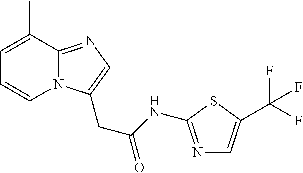 [SMILES: Cc1cccn2c(CC(=O)Nc3ncc(C(F)(F)F)s3)cnc12]

Prepared according to Method B.

LCMS Retention time (Method A): 0.983 minutes; m/z 341.0 (M+H)⁺

### Example 3-7: 2-(8-methylimidazo[1,2-a]pyridin-2-yl)-N-(5-(trifluoromethyl)thiazol-2-yl)acetamide

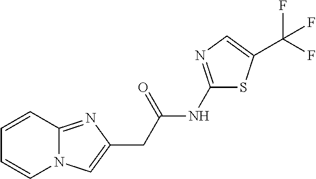 [SMILES: O=C(Cc1cn2ccccc2n1)Nc1ncc(C(F)(F)F)s1]

Prepared according to Method B.

LCMS Retention time (Method A): 1.007 minutes; m/z 341.1 (M+H)⁺

### Example 3-8: 2-(6,8-dichloroimidazo[1,2-a]pyridin-2-yl)-N-(5-(trifluoromethyl)thiazol-2-yl)acetamide

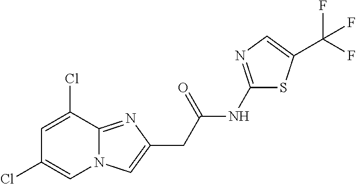 [SMILES: O=C(Cc1cn2cc(Cl)cc(Cl)c2n1)Nc1ncc(C(F)(F)F)s1]

Prepared according to Method B.

LCMS Retention time (Method A): 1.369 minutes; m/z 394.8/396.8 (M+H)⁺

### Example 3-9: 2-(6-bromo-8-methoxyimidazo[1,2-a]pyridin-2-yl)-N-(5-(trifluoromethyl)thiazol-2-yl)acetamide

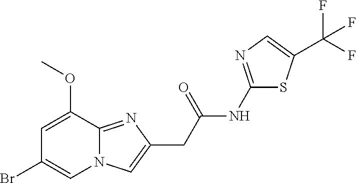 [SMILES: COc1cc(Br)cn2cc(CC(=O)Nc3ncc(C(F)(F)F)s3)nc12]

Prepared according to Method C.

LCMS Retention time (Method A): 0.974 minutes; m/z 435.0 (M+H)⁺

### Example 3-10: 2-(benzo[b]thiophen-2-yl)-N-(5-(trifluoromethyl)thiazol-2-yl)acetamide

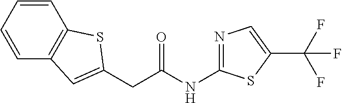 [SMILES: O=C(Cc1cc2ccccc2s1)Nc1ncc(C(F)(F)F)s1]

Prepared according to Method B.

LCMS Retention time (Method A): 1.523 minutes; m/z 343.0 (M+H)⁺

### Example 3-11: 2-(benzo[b]thiophen-2-yl)-N-(5-(trifluoromethyl)thiazol-2-yl)propanamide

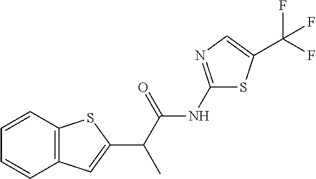 [SMILES: CC(C(=O)Nc1ncc(C(F)(F)F)s1)c1cc2ccccc2s1]

Prepared according to Method C.

LCMS Retention time (Method A): 1.423 minutes; m/z 357.0 (M+H)⁺

### Example 3-12: 2-(3-methylbenzo[b]thiophen-2-yl)-N-(5-(trifluoromethyl)thiazol-2-yl)acetamide

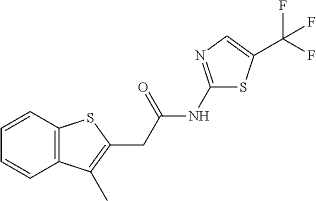 [SMILES: Cc1c(CC(=O)Nc2ncc(C(F)(F)F)s2)sc2ccccc12]

Prepared according to Method B.

LCMS Retention time (Method A): 1.488 minutes; m/z 357.0 (M+H)⁺

### Example 3-13: 2-(quinolin-5-yl)-N-(5-(trifluoromethyl)thiazol-2-yl)acetamide

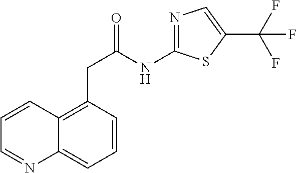 [SMILES: O=C(Cc1cccc2ncccc12)Nc1ncc(C(F)(F)F)s1]

Prepared according to Method B.

LCMS Retention time (Method A): 0.816 minutes; m/z 338.0 (M+H)⁺

### Example 3-14: 2-(quinolin-6-yl)-N-(5-(trifluoromethyl)thiazol-2-yl)acetamide

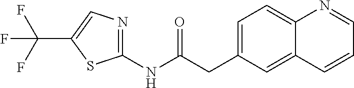 [SMILES: O=C(Cc1ccc2ncccc2c1)Nc1ncc(C(F)(F)F)s1]

Prepared according to Method B.

LCMS Retention time (Method A): 0.979 minutes; m/z 338.2 (M+H)⁺

### Example 3-15: 2-(6-methyl-1H-benzo[d]imidazol-1-yl)-N-(5-(trifluoromethyl)thiazol-2-yl)acetamide

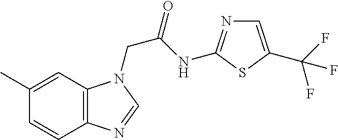 [SMILES: Cc1ccc2ncn(CC(=O)Nc3ncc(C(F)(F)F)s3)c2c1]

Prepared according to Method B.

LCMS Retention time (Method A): 0.926 minutes; m/z 341.2 (M+H)⁺

### Example 3-16: 5-oxo-1-(p-tolyl)-N-(5-(trifluoromethyl)thiazol-2-yl)pyrrolidine-3-carboxamide

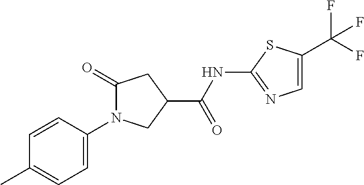 [SMILES: Cc1ccc(N2CC(C(=O)Nc3ncc(C(F)(F)F)s3)CC2=O)cc1]

Prepared according to Method B.

LCMS Retention time (Method A): 1.353 minutes; m/z 370.0 (M+H)⁺

### Example 3-17: 1-(4-fluoro-3-methylphenyl)-5-oxo-N-(5-(trifluoromethyl)thiazol-2-yl)pyrrolidine-3-carboxamide

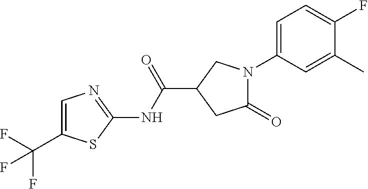 [SMILES: Cc1cc(N2CC(C(=O)Nc3ncc(C(F)(F)F)s3)CC2=O)ccc1F]

Prepared according to Method B.

LCMS Retention time (Method A): 1.231 minutes; m/z 388.2 (M+H)⁺

### Example 3-18: 1-(4-chlorophenyl)-5-oxo-N-(5-(trifluoromethyl)thiazol-2-yl)pyrrolidine-3-carboxamide

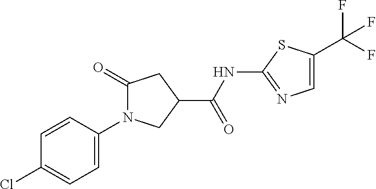 [SMILES: O=C(Nc1ncc(C(F)(F)F)s1)C1CC(=O)N(c2ccc(Cl)cc2)C1]

Prepared according to Method B.

LCMS Retention time (Method A): 1.465 minutes; m/z 390.0/392.0 (M+H)⁺

### Example 3-19: 1-(3-chlorophenyl)-5-oxo-N-(5-(trifluoromethyl)thiazol-2-yl)pyrrolidine-3-carboxamide

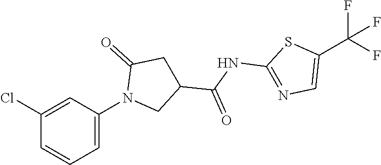 [SMILES: O=C(Nc1ncc(C(F)(F)F)s1)C1CC(=O)N(c2cccc(Cl)c2)C1]

Prepared according to Method B.

LCMS Retention time (Method A): 1.475 minutes; m/z 389.8/392.0 (M+H)⁺

### Example 3-20: 1-(2-chlorophenyl)-5-oxo-N-(5-(trifluoromethyl)thiazol-2-yl)pyrrolidine-3-carboxamide

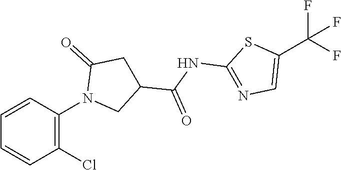 [SMILES: O=C(Nc1ncc(C(F)(F)F)s1)C1CC(=O)N(c2ccccc2Cl)C1]

Prepared according to Method B.

LCMS Retention time (Method A): 1.328 minutes; m/z 390.0/392.0 (M+H)⁺

### Example 3-21: 1-(3,5-difluorophenyl)-5-oxo-N-(5-(trifluoromethyl)thiazol-2-yl)pyrrolidine-3-carboxamide

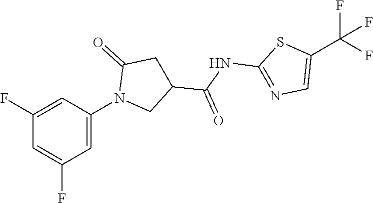 [SMILES: O=C(Nc1ncc(C(F)(F)F)s1)C1CC(=O)N(c2cc(F)cc(F)c2)C1]

Prepared according to Method B.

LCMS Retention time (Method A): 1.428 minutes; m/z 392.1 (M+H)⁺

### Example 3-22: 1-(2,5-difluorophenyl)-5-oxo-N-(5-(trifluoromethyl)thiazol-2-yl)pyrrolidine-3-carboxamide

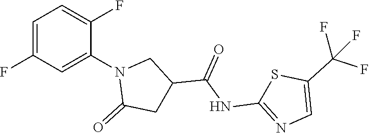 [SMILES: O=C(Nc1ncc(C(F)(F)F)s1)C1CC(=O)N(c2cc(F)ccc2F)C1]

Prepared according to Method B.

LCMS Retention time (Method A): 1.067 minutes; m/z 392.0 (M+H)⁺

### Example 3-23: 1-(3,4-difluorophenyl)-5-oxo-N-(5-(trifluoromethyl)thiazol-2-yl)pyrrolidine-3-carboxamide

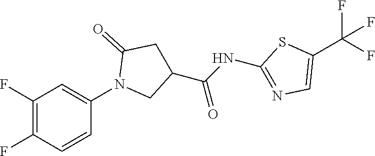 [SMILES: O=C(Nc1ncc(C(F)(F)F)s1)C1CC(=O)N(c2ccc(F)c(F)c2)C1]

Prepared according to Method B.

LCMS Retention time (Method A): 1.401 minutes; m/z 392.1 (M+H)⁺

### Example 3-24: 1-(2,3-dihydro-1H-inden-5-yl)-5-oxo-N-(5-(trifluoromethyl)thiazol-2-yl)pyrrolidine-3-carboxamide

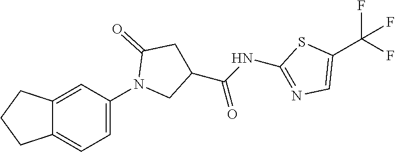 [SMILES: O=C(Nc1ncc(C(F)(F)F)s1)C1CC(=O)N(c2ccc3c(c2)CCC3)C1]

Prepared according to Method B.

LCMS Retention time (Method A): 1.510 minutes; m/z 396.0 (M+H)⁺

### Example 3-25: 1-(benzo[d][1,3]dioxol-5-yl)-5-oxo-N-(5-(trifluoromethyl)thiazol-2-yl)pyrrolidine-3-carboxamide

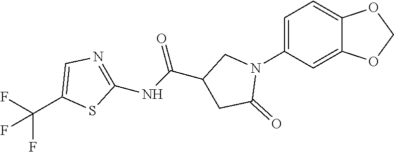 [SMILES: O=C(Nc1ncc(C(F)(F)F)s1)C1CC(=O)N(c2ccc3c(c2)OCO3)C1]

Prepared according to Method B.

LCMS Retention time (Method A): 1.317 minutes; m/z 400.0 (M+H)⁺

### Example 3-26: 1-(4-fluorophenyl)-5-oxo-N-(5-(trifluoromethyl)thiazol-2-yl)pyrrolidine-3-carboxamide

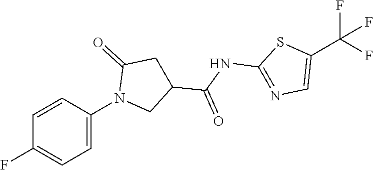 [SMILES: O=C(Nc1ncc(C(F)(F)F)s1)C1CC(=O)N(c2ccc(F)cc2)C1]

Prepared according to Method B.

LCMS Retention time (Method A): 1.124 minutes; m/z 374.0 (M+H)⁺

### Example 3-27: 1-benzyl-5-oxo-N-(5-(trifluoromethyl)thiazol-2-yl)pyrrolidine-3-carboxamide

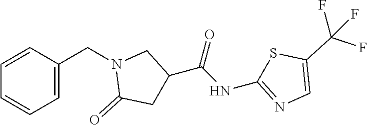 [SMILES: O=C(Nc1ncc(C(F)(F)F)s1)C1CC(=O)N(Cc2ccccc2)C1]

Prepared according to Method B.

LCMS Retention time (Method A): 1.146 minutes; m/z 370.0 (M+H)⁺

### Example 3-28: 1-(4-chlorobenzyl)-5-oxo-N-(5-(trifluoromethyl)thiazol-2-yl)pyrrolidine-3-carboxamide

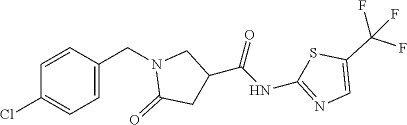 [SMILES: O=C(Nc1ncc(C(F)(F)F)s1)C1CC(=O)N(Cc2ccc(Cl)cc2)C1]

Prepared according to Method B.

LCMS Retention time (Method A): 1.393 minutes; m/z 404.0/406.0 (M+H)⁺

### Example 3-29: 1-(4-methylbenzyl)-5-oxo-N-(5-(trifluoromethyl)thiazol-2-yl)pyrrolidine-3-carboxamide

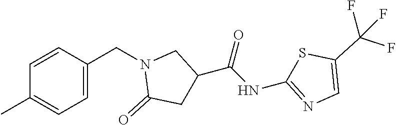 [SMILES: Cc1ccc(CN2CC(C(=O)Nc3ncc(C(F)(F)F)s3)CC2=O)cc1]

Prepared according to Method B.

LCMS Retention time (Method A): 1.397 minutes; m/z 384.1 (M+H)⁺

### Example 3-30: 2-(isoquinolin-5-yl)-N-(5-(trifluoromethyl)thiazol-2-yl)acetamide

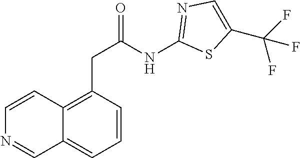 [SMILES: O=C(Cc1cccc2cnccc12)Nc1ncc(C(F)(F)F)s1]

Prepared according to Method B.

LCMS Retention time (Method A): 1.066 minutes; m/z 338.1 (M+H)⁺

### Example 3-31: 2-(isoquinolin-6-yl)-N-(5-(trifluoromethyl)thiazol-2-yl)acetamide

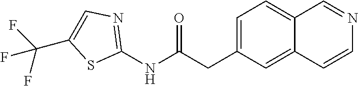 [SMILES: O=C(Cc1ccc2cnccc2c1)Nc1ncc(C(F)(F)F)s1]

Prepared according to Method B.

LCMS Retention time (Method A): 1.027 minutes; m/z 338.0 (M+H)⁺

### Example 3-32: 2-(isoquinolin-7-yl)-N-(5-(trifluoromethyl)thiazol-2-yl)acetamide

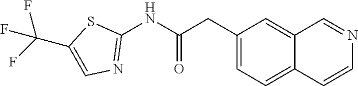 [SMILES: O=C(Cc1ccc2ccncc2c1)Nc1ncc(C(F)(F)F)s1]

Prepared according to Method C.

LCMS Retention time (Method A): 0.729 minutes; m/z 338.0 (M+H)⁺

### Example 3-33: 2-(isoquinolin-8-yl)-N-(5-(trifluoromethyl)thiazol-2-yl)acetamide

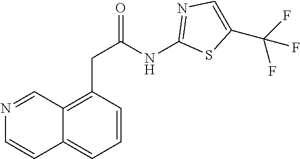 [SMILES: O=C(Cc1cccc2ccncc12)Nc1ncc(C(F)(F)F)s1]

Prepared according to Method B.

LCMS Retention time (Method A): 1.068 minutes; m/z 338.1 (M+H)⁺

### Example 3-34: 2-(isoquinolin-4-yl)-N-(5-(trifluoromethyl)thiazol-2-yl)acetamide

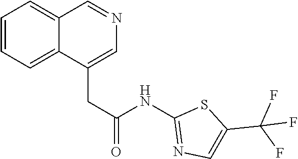 [SMILES: O=C(Cc1cncc2ccccc12)Nc1ncc(C(F)(F)F)s1]

Prepared according to Method C.

LCMS Retention time (Method A): 1.074 minutes; m/z 338.0 (M+H)⁺

### Example 3-35: 2-(quinolin-3-yl)-N-(5-(trifluoromethyl)thiazol-2-yl)propanamide

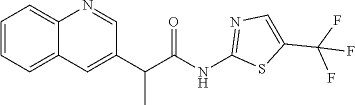 [SMILES: CC(C(=O)Nc1ncc(C(F)(F)F)s1)c1cnc2ccccc2c1]

Prepared according to Method C.

LCMS Retention time (Method A): 1.287 minutes; m/z 352.0 (M+H)⁺

### Example 3-36: 2-(quinolin-3-yl)-N-(5-(trifluoromethyl)thiazol-2-yl)acetamide

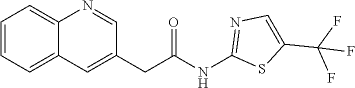 [SMILES: O=C(Cc1cnc2ccccc2c1)Nc1ncc(C(F)(F)F)s1]

Prepared according to Method B.

LCMS Retention time (Method A): 1.226 minutes; m/z 338.0 (M+H)⁺

### Example 3-37: 2-(5-methyl-1H-indol-1-yl)-N-(5-(trifluoromethyl)thiazol-2-yl)acetamide

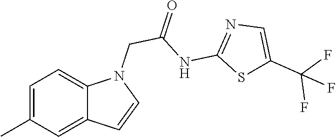 [SMILES: Cc1ccc2c(ccn2CC(=O)Nc2ncc(C(F)(F)F)s2)c1]

Prepared according to Method B.

LCMS Retention time (Method A): 1.320 minutes; m/z 340.0 (M+H)⁺

### Example 3-38: 2-(1H-pyrrolo[2,3-b]pyridin-3-yl)-N-(5-(trifluoromethyl)thiazol-2-yl)acetamide

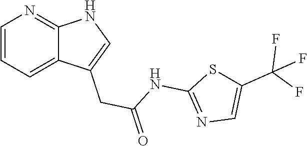 [SMILES: O=C(Cc1c[nH]c2ncccc12)Nc1ncc(C(F)(F)F)s1]

Prepared according to Method B.

LCMS Retention time (Method A): 1.143 minutes; m/z 327.1 (M+H)⁺

### Example 3-39: 2-(4-chloro-1H-pyrrolo[2,3-b]pyridin-3-yl)-N-(5-(trifluoromethyl)thiazol-2-yl)acetamide

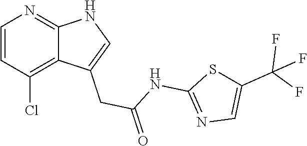 [SMILES: O=C(Cc1c[nH]c2nccc(Cl)c12)Nc1ncc(C(F)(F)F)s1]

Prepared according to Method C.

LCMS Retention time (Method A): 1.166 minutes; m/z 360.8/362.8 (M+H)⁺

### Example 3-40: 2-(1H-pyrrolo[3,2-b]pyridin-3-yl)-N-(5-(trifluoromethyl)thiazol-2-yl)acetamide

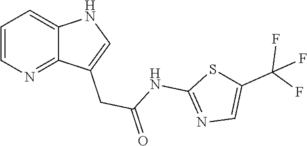 [SMILES: O=C(Cc1c[nH]c2cccnc12)Nc1ncc(C(F)(F)F)s1]

Prepared according to Method B.

LCMS Retention time (Method A): 0.898 minutes; m/z 327.9 (M+H)⁺

### Example 3-41: 2-(benzofuran-2-yl)-N-(5-(trifluoromethyl)thiazol-2-yl)acetamide

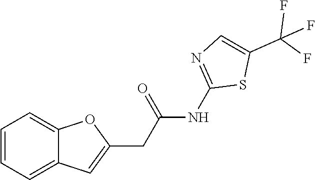 [SMILES: O=C(Cc1cc2ccccc2o1)Nc1ncc(C(F)(F)F)s1]

Prepared according to Method B.

LCMS Retention time (Method A): 1.459 minutes; m/z 327.0 (M+H)⁺

### Example 3-42: N-(5-cyclopropylthiazol-2-yl)-2-(6,7-dimethylbenzofuran-3-yl)acetamide

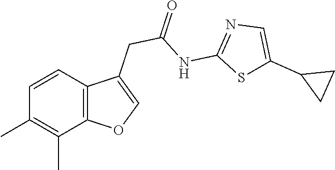 [SMILES: Cc1ccc2c(CC(=O)Nc3ncc(C4CC4)s3)coc2c1C]

Prepared according to Method A.

LCMS Retention time (Method A): 1.563 minutes; m/z 327.0 (M+H)⁺

### Example 3-43: N-(5-cyclopropylthiazol-2-yl)-2-(6-fluoroimidazo[1,2-a]pyridin-2-yl)acetamide

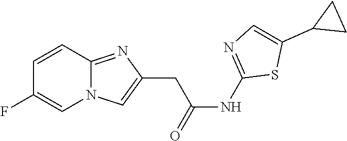 [SMILES: O=C(Cc1cn2cc(F)ccc2n1)Nc1ncc(C2CC2)s1]

Prepared according to Method A.

LCMS Retention time (Method A): 0.873 minutes; m/z 317.2 (M+H)⁺

### Example 3-44: 2-(6,7-dimethylbenzofuran-3-yl)-N-(5-methyl-1H-pyrazol-3-yl)acetamide

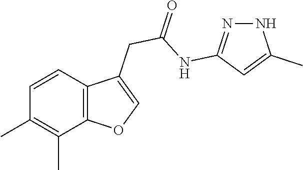

Prepared according to Method A.

LCMS Retention time (Method A): 1.284 minutes; m/z 284.2 (M+H)⁺

### Example 3-45: 2-(6,7-dimethylbenzofuran-3-yl)-N-(5-isopropyl-1H-pyrazol-3-yl)acetamide

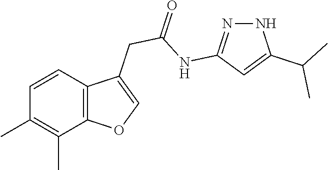 [SMILES: Cc1ccc2c(CC(=O)Nc3cc(C(C)C)[nH]n3)coc2c1C]

Prepared according to Method A.

LCMS Retention time (Method A): 1.331 minutes; m/z 312.1 (M+H)⁺

### Example 3-46: N-(5-cyclopropyl-1H-pyrazol-3-yl)-2-(6,7-dimethylbenzofuran-3-yl)acetamide

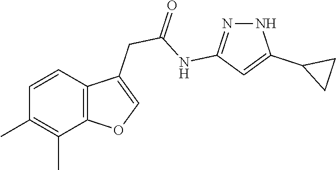 [SMILES: Cc1ccc2c(CC(=O)Nc3cc(C4CC4)[nH]n3)coc2c1C]

Prepared according to Method A.

LCMS Retention time (Method A): 1.558 minutes; m/z 310.2 (M+H)⁺

### Example 3-47: N-(5-bromo-1H-pyrazol-3-yl)-2-(6,7-dimethylbenzofuran-3-yl)acetamide

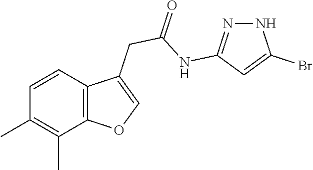 [SMILES: Cc1ccc2c(CC(=O)Nc3cc(Br)[nH]n3)coc2c1C]

Prepared according to Method B.

LCMS Retention time (Method A): 1.354 minutes; m/z 348/350.0 (M+H)⁺

### Example 3-48: 2-(6,7-dimethylbenzofuran-3-yl)-N-(5-(trifluoromethyl)-1H-pyrazol-3-yl)acetamide

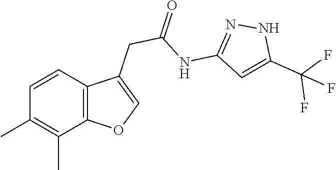 [SMILES: Cc1ccc2c(CC(=O)Nc3cc(C(F)(F)F)[nH]n3)coc2c1C]

Prepared according to Method A.

LCMS Retention time (Method A): 1.278 minutes; m/z 338.0 (M+H)⁺

### Example 3-49: N-(5-chloro-1H-pyrazol-3-yl)-2-(6,7-dimethylbenzofuran-3-yl)acetamide

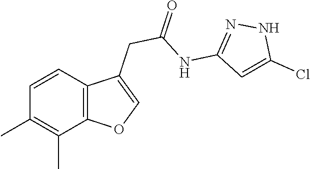 [SMILES: Cc1ccc2c(CC(=O)Nc3cc(Cl)[nH]n3)coc2c1C]

Prepared according to Method A.

LCMS Retention time (Method A): 1.293 minutes; m/z 304.0 (M+H)⁺

### Example 3-50: 2-(6,7-dimethylbenzofuran-3-yl)-N-(5-methylpyridin-2-yl)acetamide

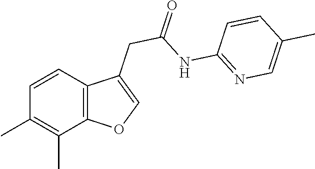

Prepared according to Method A.

LCMS Retention time (Method A): 1.337 minutes; m/z 295.2 (M+H)⁺

### Example 3-51: 2-(6,7-dimethylbenzofuran-3-yl)-N-(5-isopropylpyridin-2-yl)acetamide

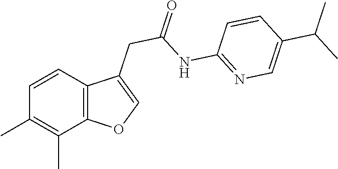 [SMILES: Cc1ccc2c(CC(=O)Nc3ccc(C(C)C)cn3)coc2c1C]

Prepared according to Method B.

LCMS Retention time (Method A): 1.449 minutes; m/z 323.1 (M+H)⁺

### Example 3-52: N-(5-cyclopropylpyridin-2-yl)-2-(6,7-dimethylbenzofuran-3-yl)acetamide

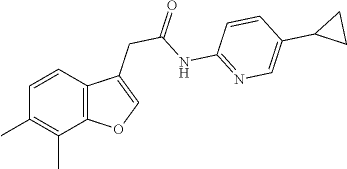 [SMILES: Cc1ccc2c(CC(=O)Nc3ccc(C4CC4)cn3)coc2c1C]

Prepared according to Method A.

LCMS Retention time (Method A): 1.531 minutes; m/z 321.2 (M+H)⁺

### Example 3-53: N-(5-bromopyridin-2-yl)-2-(6,7-dimethylbenzofuran-3-yl)acetamide

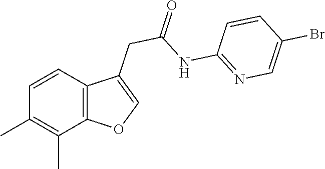 [SMILES: Cc1ccc2c(CC(=O)Nc3ccc(Br)cn3)coc2c1C]

Prepared according to Method B.

LCMS Retention time (Method A): 1.317 minutes; m/z 359.0/361 (M+H)⁺

### Example 3-54: 2-(6,7-dimethylbenzofuran-3-yl)-N-(5-(trifluoromethyl)pyridin-2-yl)acetamide

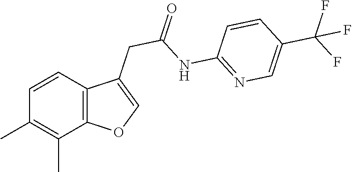 [SMILES: Cc1ccc2c(CC(=O)Nc3ccc(C(F)(F)F)cn3)coc2c1C]

Prepared according to Method B.

LCMS Retention time (Method A): 1.512 minutes; m/z 349.0 (M+H)⁺

### Example 3-55: N-(5-chloropyridin-2-yl)-2-(6,7-dimethylbenzofuran-3-yl)acetamide

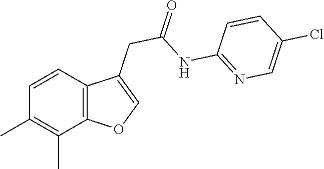 [SMILES: Cc1ccc2c(CC(=O)Nc3ccc(Cl)cn3)coc2c1C]

Prepared according to Method A.

LCMS Retention time (Method A): 1.528 minutes; m/z 315.0 (M+H)⁺

### Example 3-56: 2-(6-fluoroimidazo[1,2-a]pyridin-2-yl)-N-(5-methyl-1H-pyrazol-3-yl)acetamide

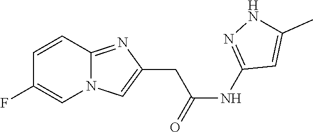 [SMILES: Cc1cc(NC(=O)Cc2cn3cc(F)ccc3n2)n[nH]1]

Prepared according to Method A.

LCMS Retention time (Method A): 0.639 minutes; m/z 274.2 (M+H)⁺

### Example 3-57: 2-(6-fluoroimidazo[1,2-a]pyridin-2-yl)-N-(5-isopropyl-1H-pyrazol-3-yl)acetamide

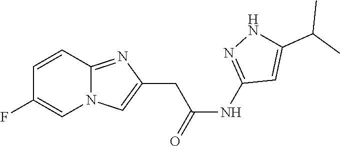 [SMILES: CC(C)c1cc(NC(=O)Cc2cn3cc(F)ccc3n2)n[nH]1]

Prepared according to Method A.

LCMS Retention time (Method A): 0.813 minutes; m/z 302.2 (M+H)⁺

### Example 3-58: N-(5-bromo-1H-pyrazol-3-yl)-2-(6-fluoroimidazo[1,2-a]pyridin-2-yl)acetamide

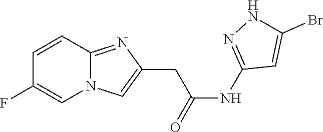 [SMILES: O=C(Cc1cn2cc(F)ccc2n1)Nc1cc(Br)[nH]n1]

Prepared according to Method C.

LCMS Retention time (Method A): 0.507 minutes; m/z 338.0/340.0 (M+H)+

### Example 3-59: 2-(6-fluoroimidazo[1,2-a]pyridin-2-yl)-N-(5-(trifluoromethyl)-1H-pyrazol-3-yl)acetamide

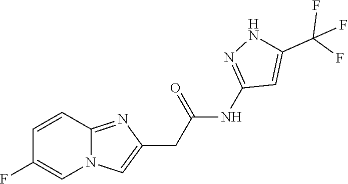 [SMILES: O=C(Cc1cn2cc(F)ccc2n1)Nc1cc(C(F)(F)F)[nH]n1]

Prepared according to Method A.

LCMS Retention time (Method A): 0.837 minutes; m/z 328.0 (M+H)⁺

### Example 3-60: N-(5-chloro-1H-pyrazol-3-yl)-2-(6-fluoroimidazo[1,2-a]pyridin-2-yl)acetamide

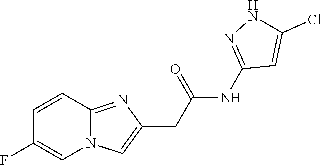 [SMILES: O=C(Cc1cn2cc(F)ccc2n1)Nc1cc(Cl)[nH]n1]

Prepared according to Method A.

LCMS Retention time (Method A): 0.526 minutes; m/z 294.0 (M+H)⁺

### Example 3-61: 2-(6-fluoroimidazo[1,2-a]pyridin-2-yl)-N-(5-isopropylpyridin-2-yl)acetamide

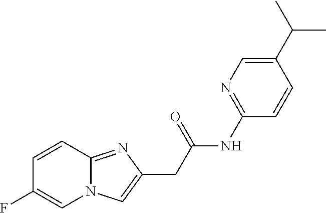 [SMILES: CC(C)c1ccc(NC(=O)Cc2cn3cc(F)ccc3n2)nc1]

Prepared according to Method B.

LCMS Retention time (Method A): 0.907 minutes; m/z 313.2 (M+H)⁺

### Example 3-62: N-(5-cyclopropylpyridin-2-yl)-2-(6-fluoroimidazo[1,2-a]pyridin-2-yl)acetamide

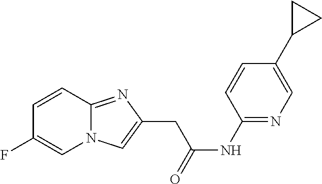 [SMILES: O=C(Cc1cn2cc(F)ccc2n1)Nc1ccc(C2CC2)cn1]

Prepared according to Method A.

LCMS Retention time (Method A): 0.873 minutes; m/z 311.1 (M+H)⁺

### Example 3-63: 3-(4-oxoquinazolin-3(4H)-yl)-N-(5-(trifluoromethyl)thiazol-2-yl)propanamide

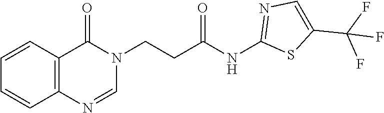 [SMILES: O=C(CCn1cnc2ccccc2c1=O)Nc1ncc(C(F)(F)F)s1]

Prepared according to Method C.

LCMS Retention time (Method A): 1.050 minutes; m/z 369.0 (M+H)⁺

### Example 3-64: 3-(6,7-dimethoxy-4-oxoquinazolin-3(4H)-yl)-N-(5-(trifluoromethyl)thiazol-2-yl)propanamide

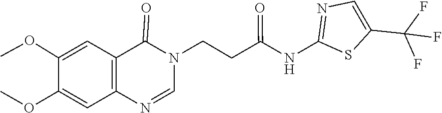 [SMILES: COc1cc2ncn(CCC(=O)Nc3ncc(C(F)(F)F)s3)c(=O)c2cc1OC]

Prepared according to Method B.

LCMS Retention time (Method A): 1.205 minutes; m/z 429.0 (M+H)⁺

### Example 3-65: 3-(6-bromo-4-oxoquinazolin-3(4H)-yl)-N-(5-(trifluoromethyl)thiazol-2-yl)propanamide

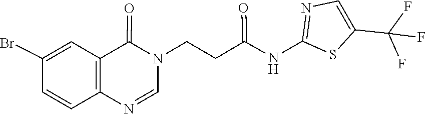 [SMILES: O=C(CCn1cnc2ccc(Br)cc2c1=O)Nc1ncc(C(F)(F)F)s1]

Prepared according to Method B.

LCMS Retention time (Method A): 1.384 minutes; m/z 448.8 (M+H)⁺

### Example 3-66: N-(5-cyclopropyl-1H-pyrazol-3-yl)-2-(6-chloroimidazo[1,2-a]pyridin-2-yl)acetamide

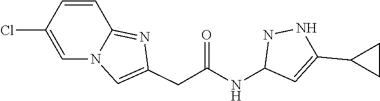 [SMILES: O=C(Cc1cn2cc(Cl)ccc2n1)NC1C=C(C2CC2)NN1]

Prepared according to Method E.

### Step 1

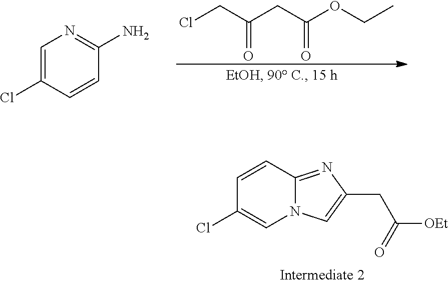

To a suspension of 5-chloropyridin-2-amine (2. g, 15.56 mmol) in EtOH (20 mL) was added ethyl 4-chloro-3-oxobutanoate (2.56 g, 15.56 mmol) at room temperature. Then the mixture was stirred at 90° C. for 16 h. After cooling to RT, the mixture was concentrated under reduce pressure. The residue was purified by column chromatography (Petroleum ether:EtOAc, 10:1, v/v) to afford Intermediate 2 as a white solid (2.5 g).

### Step 2

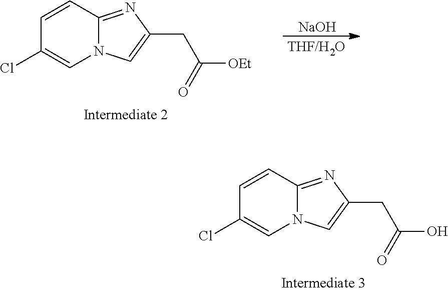 [SMILES: CCOC(=O)Cc1cn2cc(Cl)ccc2n1 ; "NaOH" ; O=C(O)Cc1cn2cc(Cl)ccc2n1]

To a suspension of Intermediate 2 (2.5 g, 10.47 mmol) in THF/H₂O (18 mL/6 mL) were added NaOH (628.43 mg, 15.71 mmol) at RT. The mixture was stirred at RT overnight. The filtration was concentrated to dryness to afford Intermediate 3 (2.0 g) as white solid, which was confirmed by LCMS and used to next step directly without further purification.

### Step 3

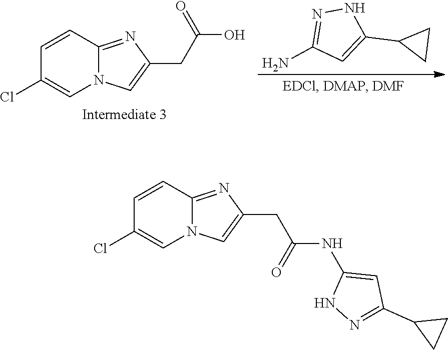 [SMILES: O=C(O)Cc1cn2cc(Cl)ccc2n1 ; Nc1cc(C2CC2)[nH]n1 ; O=C(Cc1cn2cc(Cl)ccc2n1)Nc1cc(C2CC2)n[nH]1]

To a solution of Intermediate 3 (200 mg, 949.59 mmol) in DMF (2.5 mL) were added EDCI (546.10 mg, 2.848 mol) and DMAP (348.03 mg, 2.848 mol) at RT. The mixture was stirred at RT for 5 min. The mixture was added 5-cyclopropyl-1H-pyrazol-3-amine (140.34 mg, 1.14 mmol) and stirred for 1.5 h. The resulting mixture was purified by prep-HPLC (ACN/water w/0.1% CH₂O₂, v/v=0-40%) to afford Example 3-66 (25.1 mg) as white solid.

LCMS Retention time (Method B) 7.44 min; m/z 316.10 (M+H)

### NanoBRET Fold Change Experimental Setup:

HEK293T wildtype cells are seeded 800 k cells/well (DMEM+10% FBS+1% PS) in a 6-well plate and allowed to attach for ˜7 hours followed by a transient transfection of 1.5 μg HaloTag_CDK12 Plasmid [Table 1] and 0.15 μg NanoLuc_DDB1 plasmid [Table 1] per well using PEI. Cells are incubated at 37° C., 5% CO2, harvested after 24 hours and resuspended in assay media (Opti-MEM Reduced serum media, no phenol red, 4% FBS, 1% PS). Cells are adjusted to 2.3×10⁵ cells/ml and treated with HaloTag 618 Ligand at 100 nM or DMSO. 8000 cells are seeded per well in triplicates per compound in a white-walled 384-well plate, one negative control treated with DMSO instead of HaloTag 618 Ligand is seeded in triplicates and the 384-well plate is incubated at 37° C., 5% CO₂ overnight. Compound stock solutions are prepared in Opti-MEM−/− (Opti-MEM I Reduced serum media, no phenol red) at 8× and used to treat the cells at a final concentration of 1× at 10 μM. Cells and compounds are incubated for 10 minutes at room temperature followed by addition of NanoBRET Nano-Glo substrate following manufacturer's protocols. The plate is shaken for 30 sec followed by immediate measurement of donor emission (NanoBRET Blue at 460 nm) and acceptor emission (BRET Deep Red at 647 nm) for 0.5 s.

TABLE 1
Plasmid design used for transient transfection
of HEK293T cells for NanoBRET.

| | | |
|---|---|---|
| Plasmid | HaloTag_CDK12 | Nanoluc_DDB1 |
| Tag | N-terminal HaloTag | N-terminal Nanoluc |
| | | luciferase |
| Linker | G4S | G4S |
| Protein | CDK12 (713-1052) | DDB1 (FL) |

### NanoBRET EC₅₀ Experimental Setup:

The assay setup is identical to “NanoBRET fold change experimental setup”. Instead of treating cells in duplicates with 10 μM compound, a 10-point titration starting at 20 μM is performed with a dilution factor of 1:4, covering α range of 20000 nM-0.076 nM.

### CTG Assay

KBM7 cells (wild-type or harboring α hypomorphic UBE2M mutation (Mayor-Ruiz et al., 2019)) were seeded in a white opaque 384-well plate at a density of 1000 cells per well in duplicate, in full medium (IMDM+10% FBS+1% penicillin-streptomycin). Cells were treated with compounds in a 10-point titration with a dilution factor of 1:4 (range: 0 to 20 μM). After 72 h of incubation, the cell viability was determined using the CellTiter-Glo® 2.0 reactant (Promega), pre-diluted 1:4 in mQ water. The luminescence signal was measured using the multimode plate reader EnVision 2105 (Perkin Elmer).

### Nluc-Tagged HEK293T Cell Generation

HEK293T_Nluc_CCNK-tagged cells were engineered by integrating a nanoluciferase tag at the N-terminus of the endogenous locus of CCNK using CRISPR-Cas9 technology. Briefly, 5.5 million HEK293T cells were co-transfected by PEI with a 1:1 ratio of cutting vector (sgRNA: AAGCCTACTTCAATAAATGA) and repair template (4 μg total plasmid) as previously described (Brand and Winter, 2019). The repair sequence comprises a cassette of puromycinR-P2A-HA-Nluc-(G₄S)₃ (P2A: self-cleaving peptide 2A, HA: HA-tag, (G₄S)₃: flexible linker), surrounded by 20-nucleotide microhomologies matching the genomic locus. After 3 days of incubation, the recombinant population was selected by puromycin treatment (2 μg/ml, 5-day treatment). A monoclonal population was obtained by limiting dilution and cassette integration validated by Sanger sequencing.

### Nluc Degradation (DC50)

HEK293T_Nluc_CCNK-tagged cells were seeded in a white opaque 384-well plate at a density of 8000 cells per well in duplicate, in full medium (Opti-MEM I+4% FBS+1% PS). Cells were treated with compounds in a 10-point titration (range: 0 to 10 μM, 1:4 dilution). After 4 h of incubation, Nano-Glo substrate (Promega), pre-diluted in serum-free medium (Opti-MEM I), was added to the cells (1:500 final dilution). The luminescence signal was directly measured using the multimode plate reader EnVision 2105 (Perkin Elmer).

### Western Blot Analysis of CCNK Degradation.

HEK293T cells were seeded (0.8×10⁶ cells per well in a 6-well plate) and treated with 10 μM of compound and vehicle control (DMSO) for 4 hours prior to cell lysis and protein level analysis. In short, PBS-washed cell pellets were lysed in 50 mM Tris pH 7.9, 8 M Urea, 1% CHAPS supplemented with 1% Protease Inhibitors (Protease Inhibitor Cocktail Halt™ (100×), Cat #78429, Thermo Fisher) and 0.1% Benzonase (Benzonase Nuclease 1000×, Sigma, Cat #E1014-25KU) and incubated with shaking at 1400 RPM at 10° C. for at least 30 min. Lysates were spun down at 14,000 g for 30 min at 4° C. Then 18 μg of supernatants were run and transferred for detection. Antibodies used were CCNK (1:500, Bethyl, A301-939A) and actin (1:5,000, Sigma-Aldrich, A5441). Secondary antibodies used were antimouse and antirabbit (1:10,000, Jackson ImmunoResearch 115-035-003 and 111-035-003). Change in CCNK levels was evaluated by the densitometry values normalised to actin levels using ImageJ.

### Cdk12 [High ATP] In Vitro Kinase Activity Assay

Kinase reactions (50 μl) were carried out with recombinant highly purified proteins. Cdk12 (696-1,082)/CycK (1-267) protein complex (0.5 μM) was preincubated with 50 μM of compound and vehicle control (DMSO) for 10 minutes at room temperature in kinase buffer (40 mM Tris pH 7.5, 20 mM MgCl₂, 0.1 mg/ml BSA) prior the addition of pS7-CTD substrate peptide (Ac-YSPTSP-pS-YSPTSP-pS-YSPTSP-pS-Y-PEG2-RR-amide, 95% purity, Biosyntan, Germany) (100 μM) and further 10 min incubation at room temperature. Cold ATP (to a final concentration of 1 mM) was added, and the reaction mixture was incubated for 60 min at 30° C. at 500 rpm. Kinase activity was determined using the ADP-Glo™ Kinase Assay (Promega). The luminescence signal was measured using the multimode plate reader EnVision 2105 (Perkin Elmer).

Uniprot protein codes: (Cdk12) Q9NYV4, (CycK) 075909

### REFERENCES REFERRED TO IN THE ABOVE DESCRIPTION OF THE ASSAYS

Mayor-Ruiz, Cristina & Jaeger, Martin & Bauer, Sophie & Brand, Matthias & Sin, Celine & Hanzl, Alexander & Müller, Andre & Menche, Jörg & Winter, Georg. (2019). Plasticity of the Cullin-RING Ligase Repertoire Shapes Sensitivity to Ligand-Induced Protein Degradation. Molecular Cell. 75. 849-858.e8. 10.1016/j.molcel.2019.07.013.
Brand M, Winter G E. Locus-Specific Knock-In of a Degradable Tag for Target Validation Studies. Methods Mol Biol. 2019; 1953:105-119. doi: 10.1007/978-1-4939-9145-7_7. PMID: 30912018.

### NanoBRET Fold-Change Assay Data

### Activity Classes:

“A”—greater than 4-fold increase, “B” 3-4 fold increase, “C” 2-3 fold increase, “D”—1-2 fold increase

| Example | Activity |
|---|---|
| Number | Class |
| Example 3-1 | B |
| Example 3-2 | B |
| Example 3-3 | D |
| Example 3-4 | A |
| Example 3-5 | B |
| Example 3-6 | C |
| Example 3-7 | B |
| Example 3-8 | A |
| Example 3-9 | A |
| Example 3-10 | C |
| Example 3-11 | A |
| Example 3-12 | D |
| Example 3-13 | B |
| Example 3-14 | A |
| Example 3-15 | D |
| Example 3-16 | C |
| Example 3-17 | A |
| Example 3-18 | C |
| Example 3-19 | A |
| Example 3-20 | C |
| Example 3-21 | B |
| Example 3-22 | B |
| Example 3-23 | B |
| Example 3-24 | B |
| Example 3-25 | B |
| Example 3-26 | C |
| Example 3-27 | B |
| Example 3-28 | B |
| Example 3-29 | B |
| Example 3-30 | C |
| Example 3-31 | C |
| Example 3-32 | B |
| Example 3-33 | A |
| Example 3-34 | C |
| Example 3-35 | A |
| Example 3-36 | C |
| Example 3-37 | C |
| Example 3-38 | B |
| Example 3-39 | B |
| Example 3-40 | B |
| Example 3-41 | C |
| Example 3-42 | A |
| Example 3-43 | A |
| Example 3-44 | A |
| Example 3-45 | A |
| Example 3-46 | B |
| Example 3-47 | A |
| Example 3-48 | B |
| Example 3-49 | B |
| Example 3-50 | A |
| Example 3-51 | B |
| Example 3-52 | A |
| Example 3-53 | B |
| Example 3-54 | C |
| Example 3-55 | B |
| Example 3-56 | C |
| Example 3-57 | B |
| Example 3-58 | C |
| Example 3-59 | C |
| Example 3-60 | C |
| Example 3-61 | A |
| Example 3-62 | A |
| Example 3-63 | B |
| Example 3-64 | A |
| Example 3-65 | B |
| Example 3-66 | A |

### NanoBRET EC₅₀ Assay Data

Activity Classes: “A”—EC₅₀≤0.25 μM, “B” 0.50 μM≤EC₅₀≤0.25 μM, “C” 1.0 μM≤EC₅₀<0.50 μM, “D” 10 μM≤EC₅₀<1.0 μM, “E”>10 μM

| Example | Activity |
|---|---|
| Number | Class |
| Example 3-1 | A |
| Example 3-2 | D |
| Example 3-3 | E |
| Example 3-4 | A |
| Example 3-5 | C |
| Example 3-6 | D |
| Example 3-7 | D |
| Example 3-8 | D |
| Example 3-9 | A |
| Example 3-10 | D |
| Example 3-11 | D |
| Example 3-12 | D |
| Example 3-13 | D |
| Example 3-14 | A |
| Example 3-15 | D |
| Example 3-16 | D |
| Example 3-17 | B |
| Example 3-18 | D |
| Example 3-19 | B |
| Example 3-20 | D |
| Example 3-21 | D |
| Example 3-22 | E |
| Example 3-23 | D |
| Example 3-24 | D |
| Example 3-25 | D |
| Example 3-26 | D |
| Example 3-27 | D |
| Example 3-28 | D |
| Example 3-29 | D |
| Example 3-30 | D |
| Example 3-31 | D |
| Example 3-32 | D |
| Example 3-33 | D |
| Example 3-34 | D |
| Example 3-35 | A |
| Example 3-36 | B |
| Example 3-37 | D |
| Example 3-38 | C |
| Example 3-39 | D |
| Example 3-40 | B |
| Example 3-41 | D |
| Example 3-42 | C |
| Example 3-43 | A |
| Example 3-44 | A |
| Example 3-45 | B |
| Example 3-46 | D |
| Example 3-47 | B |
| Example 3-48 | B |
| Example 3-49 | D |
| Example 3-50 | A |
| Example 3-51 | C |
| Example 3-52 | A |
| Example 3-53 | E |
| Example 3-54 | D |
| Example 3-55 | D |
| Example 3-56 | D |
| Example 3-57 | D |
| Example 3-58 | D |
| Example 3-59 | E |
| Example 3-60 | D |
| Example 3-61 | D |
| Example 3-62 | A |
| Example 3-63 | E |
| Example 3-64 | E |
| Example 3-65 | D |
| Example 3-66 | A |

### KBM7 CTG EC₅₀ Assay Data (UBE2M Wild-Type)

### Activity Classes:

“A”—EC₅₀≤0.25 μM, “B” 0.50 μM EC₅₀<0.25 μM, “C” 1.0 μM≤EC₅₀<0.50 μM, “D” 10 μM≤EC₅₀<1.0 μM, “E”>10 μM

| Example | Activity |
|---|---|
| Number | Class |
| Example 3-1 | A |
| Example 3-2 | A |
| Example 3-3 | A |
| Example 3-4 | A |
| Example 3-5 | A |
| Example 3-6 | A |
| Example 3-7 | A |
| Example 3-8 | A |
| Example 3-9 | A |
| Example 3-10 | A |
| Example 3-11 | A |
| Example 3-12 | E |
| Example 3-13 | D |
| Example 3-14 | A |
| Example 3-15 | D |
| Example 3-16 | B |
| Example 3-17 | A |
| Example 3-18 | A |
| Example 3-19 | A |
| Example 3-20 | B |
| Example 3-21 | A |
| Example 3-22 | A |
| Example 3-23 | A |
| Example 3-24 | A |
| Example 3-25 | B |
| Example 3-26 | D |
| Example 3-27 | A |
| Example 3-28 | A |
| Example 3-29 | C |
| Example 3-30 | E |
| Example 3-31 | D |
| Example 3-32 | A |
| Example 3-33 | A |
| Example 3-34 | D |
| Example 3-35 | A |
| Example 3-36 | A |
| Example 3-37 | D |
| Example 3-38 | C |
| Example 3-39 | D |
| Example 3-40 | A |
| Example 3-41 | D |
| Example 3-42 | A |
| Example 3-43 | A |
| Example 3-44 | A |
| Example 3-45 | A |
| Example 3-46 | C |
| Example 3-47 | A |
| Example 3-48 | A |
| Example 3-49 | A |
| Example 3-50 | B |
| Example 3-51 | D |
| Example 3-52 | A |
| Example 3-53 | A |
| Example 3-54 | A |
| Example 3-55 | B |
| Example 3-56 | B |
| Example 3-57 | A |
| Example 3-58 | A |
| Example 3-59 | B |
| Example 3-60 | D |
| Example 3-61 | A |
| Example 3-62 | A |
| Example 3-63 | A |
| Example 3-64 | A |
| Example 3-65 | C |
| Example 3-66 | A |

### KBM7 CTG EC₅₀ Assay Data (UBE2M Knock-Out)

### Activity Classes:

“A”—EC₅₀≤0.25 μM, “B” 0.50 μM≤EC₅₀<0.25 μM, “C” 1.0 μM≤EC₅₀<0.50 μM, “D” 10 μM≤EC₅₀<1.0 μM, “E”>10 μM

| Example | Activity |
|---|---|
| Number | Class |
| Example 3-1 | C |
| Example 3-2 | C |
| Example 3-3 | D |
| Example 3-4 | D |
| Example 3-5 | B |
| Example 3-6 | D |
| Example 3-7 | D |
| Example 3-8 | A |
| Example 3-9 | A |
| Example 3-10 | D |
| Example 3-11 | A |
| Example 3-12 | E |
| Example 3-13 | E |
| Example 3-14 | A |
| Example 3-15 | E |
| Example 3-16 | E |
| Example 3-17 | B |
| Example 3-18 | E |
| Example 3-19 | A |
| Example 3-20 | E |
| Example 3-21 | D |
| Example 3-22 | E |
| Example 3-23 | E |
| Example 3-24 | D |
| Example 3-25 | E |
| Example 3-26 | E |
| Example 3-27 | E |
| Example 3-28 | D |
| Example 3-29 | E |
| Example 3-30 | E |
| Example 3-31 | E |
| Example 3-32 | D |
| Example 3-33 | D |
| Example 3-34 | E |
| Example 3-35 | A |
| Example 3-36 | A |
| Example 3-37 | E |
| Example 3-38 | E |
| Example 3-39 | E |
| Example 3-40 | E |
| Example 3-41 | E |
| Example 3-42 | A |
| Example 3-43 | A |
| Example 3-44 | B |
| Example 3-45 | A |
| Example 3-46 | E |
| Example 3-47 | C |
| Example 3-48 | C |
| Example 3-49 | D |
| Example 3-50 | E |
| Example 3-51 | E |
| Example 3-52 | D |
| Example 3-53 | D |
| Example 3-54 | D |
| Example 3-55 | D |
| Example 3-56 | E |
| Example 3-57 | D |
| Example 3-58 | E |
| Example 3-59 | E |
| Example 3-60 | E |
| Example 3-61 | E |
| Example 3-62 | B |
| Example 3-63 | D |
| Example 3-64 | C |
| Example 3-65 | E |
| Example 3-66 | A |

The calculated ratio between the anti-proliferative activity in KBM7 wild-type versus UBE2M knock-out cells is indicative of the dependence of cytotoxicity on E3 ligases and a glue degrader mechanism of action.

| Example | Ratio UBE2M |
|---|---|
| Number | WT to KO |
| Example 3-1 | 25.3 |
| Example 3-2 | 12.7 |
| Example 3-3 | 23.5 |
| Example 3-4 | 82.6 |
| Example 3-5 | 142.0 |
| Example 3-6 | 129.4 |
| Example 3-7 | 42.4 |
| Example 3-8 | 51.5 |
| Example 3-9 | 2.0 |
| Example 3-10 | 23.6 |
| Example 3-11 | 9.4 |
| Example 3-12 | >10 |
| Example 3-13 | >4 |
| Example 3-14 | 49.3 |
| Example 3-15 | >4 |
| Example 3-16 | >10 |
| Example 3-17 | 17.0 |
| Example 3-18 | >10 |
| Example 3-19 | 9.1 |
| Example 3-20 | >10 |
| Example 3-21 | 44.5 |
| Example 3-22 | >10 |
| Example 3-23 | >10 |
| Example 3-24 | 23.8 |
| Example 3-25 | >10 |
| Example 3-26 | >5 |
| Example 3-27 | >10 |
| Example 3-28 | 42.7 |
| Example 3-29 | >16 |
| Example 3-30 | >1 |
| Example 3-31 | >4 |
| Example 3-32 | 115.9 |
| Example 3-33 | 64.7 |
| Example 3-34 | >3 |
| Example 3-35 | 15.1 |
| Example 3-36 | 41.3 |
| Example 3-37 | >4 |
| Example 3-38 | >10 |
| Example 3-39 | >4 |
| Example 3-40 | >200 |
| Example 3-41 | >2 |
| Example 3-42 | 8.5 |
| Example 3-43 | 39.0 |
| Example 3-44 | 34.4 |
| Example 3-45 | 18.6 |
| Example 3-46 | >10 |
| Example 3-47 | 15.7 |
| Example 3-48 | 15.7 |
| Example 3-49 | 37.3 |
| Example 3-50 | >10 |
| Example 3-51 | >8 |
| Example 3-52 | 27.0 |
| Example 3-53 | 22.7 |
| Example 3-54 | 24.7 |
| Example 3-55 | 9.2 |
| Example 3-56 | >10 |
| Example 3-57 | 146.3 |
| Example 3-58 | >10 |
| Example 3-59 | >10 |
| Example 3-60 | >9 |
| Example 3-61 | >20 |
| Example 3-62 | 50.8 |
| Example 3-63 | 13.9 |
| Example 3-64 | 21.9 |
| Example 3-65 | >13 |
| Example 3-66 | 388.0 |

### NanoLuc-CCNK Degradation Assay

“A”—DC₅₀≤0.10 μM, “B” 0.50 μM≤DC₅₀<0.10 μM, “C” 1.0 μM≤DC₅₀<0.50 μM, “D” 10 μM≤DC₅₀<1.0 μM, “E”>10 μM

| Example | Activity |
|---|---|
| Number | Class |
| Example 3-1 | B |
| Example 3-2 | A |
| Example 3-3 | E |
| Example 3-4 | B |
| Example 3-5 | A |
| Example 3-6 | D |
| Example 3-7 | E |
| Example 3-8 | A |
| Example 3-9 | A |
| Example 3-10 | A |
| Example 3-11 | A |
| Example 3-12 | E |
| Example 3-13 | E |
| Example 3-14 | A |
| Example 3-15 | E |
| Example 3-16 | C |
| Example 3-17 | A |
| Example 3-18 | D |
| Example 3-19 | A |
| Example 3-20 | E |
| Example 3-21 | B |
| Example 3-22 | E |
| Example 3-23 | E |
| Example 3-24 | B |
| Example 3-25 | E |
| Example 3-26 | E |
| Example 3-27 | A |
| Example 3-28 | C |
| Example 3-29 | E |
| Example 3-30 | E |
| Example 3-31 | E |
| Example 3-32 | A |
| Example 3-33 | E |
| Example 3-34 | E |
| Example 3-35 | A |
| Example 3-36 | A |
| Example 3-37 | E |
| Example 3-38 | E |
| Example 3-39 | D |
| Example 3-40 | D |
| Example 3-41 | D |
| Example 3-42 | A |
| Example 3-43 | A |
| Example 3-44 | B |
| Example 3-45 | A |
| Example 3-46 | D |
| Example 3-47 | A |
| Example 3-48 | B |
| Example 3-49 | B |
| Example 3-50 | D |
| Example 3-51 | D |
| Example 3-52 | B |
| Example 3-53 | B |
| Example 3-54 | E |
| Example 3-55 | C |
| Example 3-56 | E |
| Example 3-57 | B |
| Example 3-58 | E |
| Example 3-59 | E |
| Example 3-60 | E |
| Example 3-61 | B |
| Example 3-62 | A |
| Example 3-63 | B |
| Example 3-64 | A |
| Example 3-65 | B |
| Example 3-66 | A |

The results of Western blot analysis of CCNTK degradation and percentage CCNK remaining 4 h 10 micromolar treatment as evaluated by the densitometry values normalised to actin levels using JmageJ are shown in FIGS. 16 and 17.

It is believed that Example 3-66 is particularly exceptional in the CCNK degradation assays (NanoBRET and NanoLuc) because it contains the 6-chloroimidazo[1,2-a]pyridin-2-yl radical positioned towards the DDB1-Cdk12 interface, leading to a particularly strong stabilizing interaction. Additionally, presence of the 5-cyclopropyl-1H-pyrazol-3-yl radical gives optimal hydrophobic interactions with the buried part of the ATP binding pocket. It is believed that these interactions lead to enhanced CCNK degradation, leading to more extensive cancer cell death (as observed in the KBM7 cytotoxicity assay). The very strong dependence on UBE2M-mediated degradation is apparent due to the significantly lower activity in the KBM7 UBE2M mutant cell line.

Similarly, Example 3-9 is also a particularly potent anticancer agent. The 6-bromo-8-methoxyimidazo[1,2-a]pyridin-2-yl radical leads to enhanced ternary complex formation as evidenced by the exceptional activity in the NanoLuc and NanoBRET CCNK degradation assays, leading to extremely potent activity in the KBM7 cell assay. The 5-(trifluoromethyl)thiazol-2-yl) radical forms, like the 5-cyclopropyl-1H-pyrazol-3-yl radical mentioned above, optimal interactions with the hydrophobic ATP-binding pocket.

Example 3-45 shows extremely high dependence on UBE2M-dependent cytotoxicity, as shown by the ratio of UBE2M wild-type to mutant cytotoxicity data. It is believed that this manifests because of increased decoupling of the effect of direct Cdk12 inhibition from the degrader mechanism (kinase activity assay data) due to the presence of the 5-isopropyl-1H-pyrazol-3-yl radical (not optimal for Cdk12 inhibition) versus the presence of the 6-fluoroimidazo[1,2-a]pyridin-2-yl radical (preferred for interactions between Cdk12 and DDB1).

Example 3-36 shows extremely potent activity in cytotoxicity assays (KBM7) and good activity in assays of ternary complex formation. In contrast to Example 3-45, the 5-(trifluoromethyl)thiazol-2-yl) radical is optimal for Cdk12 binding, whilst the 2-(quinolin-3-yl)-radical forms optimal interactions at the glue interface (but is not optimal for Cdk12 inhibition), as shown by the weak Cdk12 inhibition at physiological ATP concentrations (kinase activity assay). The strong dependence on UBE2M-mediated degradation is apparent due to the significantly (40-fold) lower activity in the KBM7 UBE2M mutant cell line.

Example 3-14 shows extremely potent activity in cytotoxicity assays (KBM7) and extremely high activity in assays of ternary complex formation (NanoLuc and NanoBRET assays). The 5-(trifluoromethyl)thiazol-2-yl) radical is optimal for Cdk12 binding, whilst the 2-(quinolin-6-yl) radical forms superior interactions to stabilize the binding at the DDB1-Cdk12 interface.

The strong dependence on UBE2M-mediated degradation is apparent due to the significantly (˜50-fold) lower activity in the KBM7 UBE2M mutant cell line.

Example 3-5 is particularly exceptional in the CCNK degradation assays (NanoBRET and NanoLuc assays) due to the preferred 6-fluoroimidazo[1,2-a]pyridin-2-yl radical positioned towards the DDB1-Cdk12 interface (similar to Example 3-45). This leads to a particularly strong stabilizing interaction. Additionally, presence of the 5-(trifluoromethyl)thiazol-2-yl) radical gives optimal hydrophobic interactions with the buried part of the ATP binding pocket. It is believed that these interactions lead to enhanced CCNK degradation, leading to more extensive cancer cell death (as observed in the KBM7 cytotoxicity assay). The very strong dependence on UBE2M-mediated degradation is apparent due to the significantly lower activity in the KBM7 UBE2M mutant cell line (ratio of 142).

Example 3-43 is exceptional in the CCNK degradation assays (NanoBRET and NanoLuc) because it contains the 6-fluoroimidazo[1,2-a]pyridin-2-yl radical positioned towards the DDB1-Cdk12 interface, leading to particularly strong stabilizing interactions. Additionally, presence of the 5-cyclopropylthiazol-2-yl radical gives optimal hydrophobic interactions with the buried part of the ATP binding pocket. Combination of these features leads to extremely potent activity in cell proliferation assays (KBM7 wild-type versus mutant), with strong dependence on UBE2M-dependent CCNK degradation (activity ratio of ˜40).

1. A compound of the following formula (I) or a stereoisomer, tautomer, pharmaceutically acceptable salt, solvate or prodrug thereof: 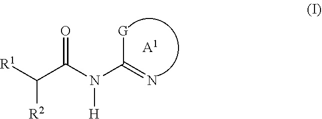
wherein
R¹ is selected from optionally substituted bicyclic aryl and optionally substituted bicyclic heteroaryl,
R² is selected from hydrogen and alkyl,
A¹ is optionally substituted five- or six-membered monocyclic heteroaryl, and
G is a ring atom selected from oxygen, sulfur, carbon and nitrogen, with the proviso that the following compounds are disclaimed:

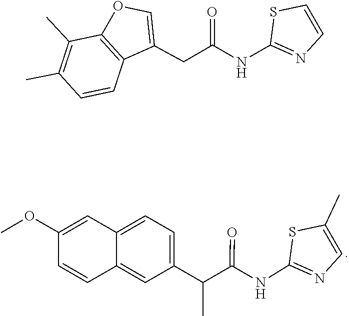 [SMILES: Cc1ccc2c(CC(=O)Nc3nccs3)coc2c1C ; COc1ccc2cc(C(C)C(=O)Nc3ncc(C)s3)ccc2c1]

2. The compound according to claim 1, wherein R¹ is selected from optionally substituted naphthyl, benzothienyl, benzofuranyl, isobenzofuranyl, chromenyl, indolizinyl, isoindolyl, indolyl, indazolyl, purinyl, isoquinolyl, quinolyl, phthalazinyl, naphthyridinyl, quinoxalinyl, cinnolinyl, 1,2-benzoisoxazol-3-yl, benzothiazolyl, benzoxazolyl, benzisoxazolyl, benzimidazolyl, coumarinyl, and chromonyl.

3. The compound according to claim 1, wherein R² is selected from hydrogen and methyl.

4. The compound according to claim 1, wherein A¹ is selected from optionally substituted thiazolyl, pyrazolyl and pyridinyl.

5. The compound claim 1, wherein G is selected from O, S, CH, N, NH and N(alkyl).

6. A compound of the following formula (IV) or (V), or a stereoisomer, tautomer, pharmaceutically acceptable salt, solvate or prodrug thereof: 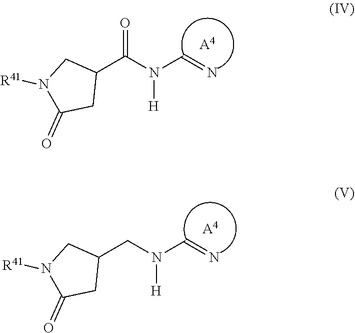
wherein
R⁴¹ is selected from -(optionally substituted aryl), -(optionally substituted heteroaryl), -(optionally substituted alkylene)-(optionally substituted aryl) and -(optionally substituted alkylene)-(optionally substituted heteroaryl), and
A⁴ is optionally substituted monocyclic or bicyclic heteroaryl,
with the proviso that the following compounds are disclaimed:

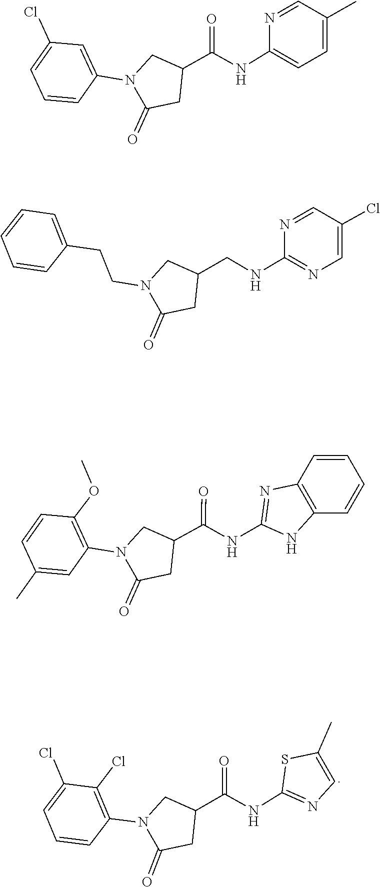

7. The compound according to claim 6, wherein R⁴¹ is selected from -(optionally substituted aryl) and -(optionally substituted alkylene)-(optionally substituted aryl).

8. The compound according to claim 6, wherein A⁴ is selected from optionally substituted pyrrolyl, imidazolyl, pyrazolyl, pyridyl, pyrazinyl, pyrimidinyl, pyridazinyl, indolizinyl, isoindolyl, indolyl, indazolyl, isoquinolyl, quinolyl, thiazolyl, isothiazolyl, phenothiazinyl, oxazolyl, isoxazolyl, furazanyl, benzothiazolyl, benzoxazolyl, benzisoxazolyl and benzoimidazolyl.

9. A compound of the following formula (II) or a stereoisomer, tautomer, pharmaceutically acceptable salt, solvate or prodrug thereof: 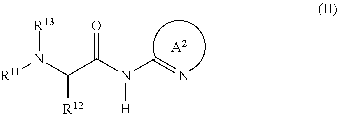
wherein
R¹¹ is selected from -(optionally substituted aryl), -(optionally substituted heteroaryl), —C(O)-(optionally substituted aryl) and —C(O)-(optionally substituted heteroaryl),
R¹² is selected from hydrogen and alkyl,
R¹³ is selected from hydrogen and alkyl,
wherein R¹² and R¹³ are optionally linked to form, together with the nitrogen and the carbon to which they are connected, an optionally substituted heterocycloalkyl group, wherein the optionally substituted heterocycloalkyl group does not contain any oxygen or sulfur in the ring,
A² is an optionally substituted five- to ten-membered heteroaryl group,
with the proviso that the following compounds are disclaimed:

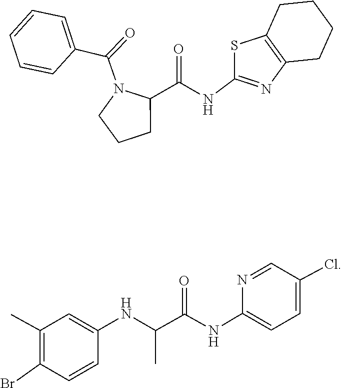 [SMILES: O=C(Nc1nc2c(s1)CCCC2)C1CCCN1C(=O)c1ccccc1 ; Cc1cc(NC(C)C(=O)Nc2ccc(Cl)cn2)ccc1Br]

10-12. (canceled)

13. A compound of the following formula (III), or a stereoisomer, tautomer, pharmaceutically acceptable salt, solvate or prodrug thereof: 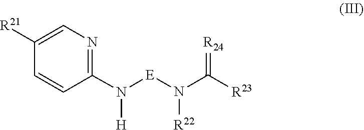
wherein
E is a linear C₂₋₄ alkylene group wherein one or more of the CH₂ units are each optionally replaced by any one independently selected from S, O and NH, wherein the linear C₂₋₄ alkylene group is optionally substituted with 1, 2, 3 or 4 substituents independently selected from ═O, —OH, -Hal, —C₁₋₆ alkyl, and —C₁₋₆ haloalkyl,
R²¹ is selected from -halogen, —NO₂, —C(═O)H, —C(═O)R²⁶, —COOH, —C(═O)OR²⁷, —CF₃ and —CN, wherein R²⁶ and R²⁷ are each independently selected from alkyl and haloalkyl,
R²² is selected from hydrogen and alkyl,
R²³ is selected from optionally substituted aryl and optionally substituted heteroaryl,
R²⁴ is selected from O and NR²⁵, wherein R²⁵ is selected from alkyl or haloalkyl,
wherein R²² is optionally linked to R²³, or R²³ is optionally linked to R²⁵,
with the proviso that the following compounds are disclaimed:

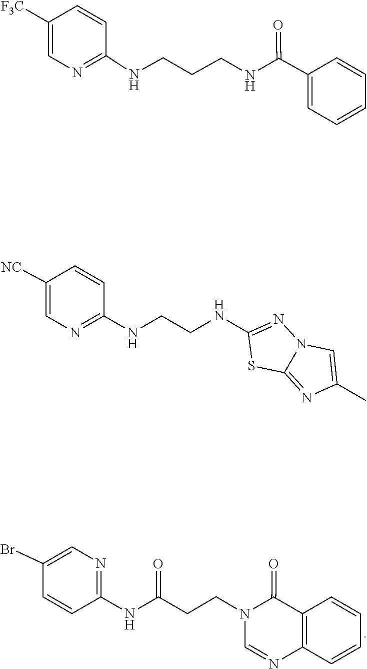 [SMILES: O=C(NCCCNc1ccc(C(F)(F)F)cn1)c1ccccc1 ; Cc1cn2nc(NCCNc3ccc(C#N)cn3)sc2n1 ; O=C(CCn1cnc2ccccc2c1=O)Nc1ccc(Br)cn1]

14-23. (canceled)

24. A pharmaceutical composition, comprising
(a) a compound of the following formula (I) or a stereoisomer, tautomer, pharmaceutically acceptable salt, solvate or prodrug thereof: 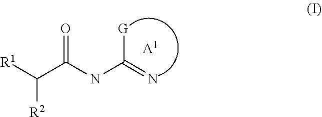
wherein
R¹ is selected from optionally substituted bicyclic aryl and optionally substituted bicyclic heteroaryl,
R² is selected from hydrogen and alkyl,
A¹ is optionally substituted five- or six-membered monocyclic heteroaryl, and
G is a ring atom selected from oxygen, sulfur, carbon and nitrogen; or
(b) a compound of the following formula (IV) or (V), or a stereoisomer, tautomer, pharmaceutically acceptable salt, solvate or prodrug thereof: 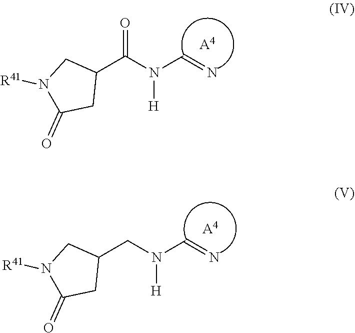
wherein
R⁴¹ is selected from -(optionally substituted aryl), -(optionally substituted heteroaryl), -(optionally substituted alkylene)-(optionally substituted aryl) and -(optionally substituted alkylene)-(optionally substituted heteroaryl), and
A⁴ is optionally substituted monocyclic or bicyclic heteroaryl,
and a pharmaceutically acceptable diluent, excipient or carrier.

25-39. (canceled)

40. A method of treating or preventing a disease comprising administering an effective amount of a compound, wherein the compound is
(a) a compound of the following formula (I) or a stereoisomer, tautomer, pharmaceutically acceptable salt, solvate or prodrug thereof: 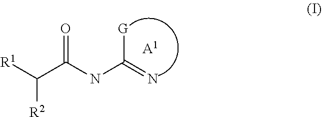
wherein
R¹ is selected from optionally substituted bicyclic aryl and optionally substituted bicyclic heteroaryl,
R² is selected from hydrogen and alkyl,
A¹ is optionally substituted five- or six-membered monocyclic heteroaryl, and
G is a ring atom selected from oxygen, sulfur, carbon and nitrogen; or
(b) a compound of the following formula (IV) or (V), or a stereoisomer, tautomer, pharmaceutically acceptable salt, solvate or prodrug thereof: 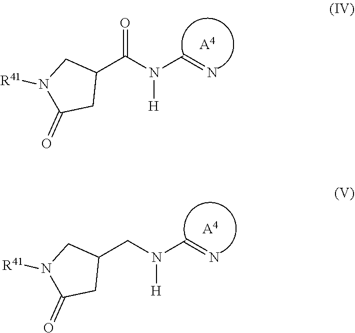
wherein
R⁴¹ is selected from -(optionally substituted aryl), -(optionally substituted heteroaryl), -(optionally substituted alkylene)-(optionally substituted aryl) and -(optionally substituted alkylene)-(optionally substituted heteroaryl), and
A⁴ is optionally substituted monocyclic or bicyclic heteroaryl.

41. The method of claim 40, wherein the disease is cancer, a metabolic disorder, a neurologic disorder or an infectious disease and the subject is a patient having cancer, a metabolic disorder, a neurologic disorder or an infectious disease.

42. The method of claim 41, wherein the disease is cancer and the subject is a patient having cancer.

43-45. (canceled)

46. The method of claim 42, wherein the cancer is selected from the group consisting of leukemia, a chronic leukemia, adenoid cystic carcinoma, osteosarcoma, ovarian cancer, Ewings sarcoma, lung adenocarcinoma and prostate cancer, lymphoma, neuroblastoma, a gastrointestinal cancer, endometrial cancer, medulloblastoma, prostate cancer, esophagus cancer, breast cancer, thyroid cancer, meningioma, liver cancer, colorectal cancer, pancreatic cancer, chondrosarcoma, osteosarcoma, and kidney cancer.

47. The method of claim 46, wherein the cancer is leukemia.

48. A method of treating or preventing a disease comprising administering an effective amount of the pharmaceutical composition of claim 24.

49. The compound of claim 5, wherein G is selected from O, S, NH and N(alkyl).

50. The compound of claim 49, wherein G is O or S.
